(19) United States
(12) Patent Application Publication
(10) Pub. No.: US 2023/0295490 A1
(43) Pub. Date: Sep. 21, 2023

(54) LIGHT-EMITTING NANOPARTICLES AND LIGHT-EMITTING LABELING MATERIAL FOR PATHOLOGICAL DIAGNOSIS

(71) Applicant: Konica Minolta, Inc., Chiyoda-ku, Tokyo (JP)

(72) Inventors: Yasuo MIYATA, Yokohama-shi, Kanagawa (JP); Kenichi TABATA, Tama-shi, Tokyo (JP); Kazuma ODA, Hachioji-shi, Tokyo (JP); Kohei OGINO, Hino-shi, Tokyo (JP)

(21) Appl. No.: 18/011,244

(22) PCT Filed: Jun. 18, 2021
(86) PCT No.: PCT/JP2021/023203

§ 371 Date: Dec. 19, 2022

(30) Foreign Application Priority Data
Jun. 25, 2020 (JP) 2020-109301

(51) Int. Cl. C09K 11/06; G01N 33/58; C09K 11/02

(52) U.S. Cl. CPC C09K 11/06; G01N 33/582; G01N 33/587; C09K 11/02; C09K 2211/1022; C09K 2211/1014; C09K 2211/1007

(57) ABSTRACT

The present invention addresses the problem of providing a luminescent labeling material for pathological diagnosis and luminescent nanoparticles for enabling high-sensitivity imaging that can avoid the negative impact of auto-fluorescence of cells on bioimaging. Provided are luminescent nanoparticles containing a luminescent dye, wherein the luminescent dye is a compound having a specific structure represented by Formula (1), and the luminescent nanoparticles have at least one of delayed luminesence or long Stokes shift luminescence.

### TECHNICAL FIELD

The present invention relates to luminescent nanoparticles and luminescent labeling materials for pathological diagnosis, and more particularly to luminescent nanoparticles and luminescent labeling materials for pathological diagnosis for enabling high sensitivity imaging that can avoid the adverse effects of cell autofluorescence on bioimaging.

### BACKGROUND TECHNOLOGY

In “bioimaging” using luminescent dyes, it is a major issue to separate the luminescence of the luminescent dye from the autofluorescence of the cell for high-sensitivity imaging that can position and quantify the protein by the bright spots of nanoparticles in the imaging image. The term “bioimaging” refers to the following. Specifically adsorbing a luminescent (e.g., fluorescent) probe, such as a microscopic luminescent particle, to a target such as a cell or protein, and then using the luminescence from the luminescent probe, the target structure and their position or movement within a living body is observed.

One means of avoiding the negative effects of autofluorescence on bioimaging is to use phenomena such as near-infrared luminescence, long Stokes shift luminescence and delayed luminescence. Although near-infrared luminescence is the most widely used method among them, it is difficult to achieve multicolor staining, which is important for the analysis of intracellular biomolecules that work cooperatively.

Long Stokes shift luminescence is an effective means of avoiding the wavelength region where the luminescence intensity (also referred to as “brightness”) of the autofluorescence of cells is high by providing a large difference between the excitation wavelength and the emission wavelength. However, the long Stokes shift luminescence above 100 nm has a problem of low luminescence as typified by the general exciplex luminescence.

Delayed luminescence includes phosphorescence luminescence, heat-activated delayed fluorescence, and exciplex luminescence, and can emit light for a longer period of time, around 1 microsecond, than cell autofluorescence, which disappears around 10 nanoseconds. Delayed luminescence is a promising method for luminescence imaging in the time period after the disappearance of cell autofluorescence.

Non-patent documents 1 and 2 disclose time-resolved cell imaging techniques using nanoparticles with dyes that exhibit heat-active delayed fluorescence. In time-resolved imaging, the luminescence intensity of the delayed luminescence itself is necessary for imaging at the time after the disappearance of the autofluorescence of the cell. However, the technique disclosed in non-patent document 1 has a problem with the quantum yield of luminescence, and the technique disclosed in non-patent document 2 has a problem with low luminescence intensity of delayed fluorescence. Therefore, it is necessary to further improve the luminescence to satisfy the above-described high-sensitivity imaging application.

Therefore, both long Stokes shift luminescence and delayed luminescence have luminescence problems for the above-mentioned high-sensitivity imaging applications. As a workaround, there is a method to accumulate the number of imaging times, but this method causes degradation of cells and luminescent dyes due to repeated irradiation of excitation pulsed light, which leads to a decrease in imaging accuracy. Therefore, it is necessary to increase the luminance (=absorbance×quantum yield) of the luminescent nanoparticles themselves in order to shorten the integration time of imaging. To increase the luminance of the nanoparticles, the quantum yield must be maintained while improving the absorbance in the nanoparticles.

To improve the absorbance (=molar absorption coefficient×molar concentration) of a luminescent dye, there are methods of increasing the molar absorption coefficient of the dye itself or increasing the concentration of the dye in the particles. Here, to maintain the luminescence of the long Stokes shift luminescence or delayed luminescence dyes, it is necessary to maintain the energy level of the high lowest triplet excited state (T₁) where the excited ligand moiety, donor moiety (electron-donating group) and acceptor moiety (electron-withdrawing group) suppress quenching phenomena. Therefore, the extension of the π-conjugated system that lowers the T₁ level must be avoided, and as a result, a significant increase in the molar absorption coefficient is difficult to achieve from a molecular design point of view.

In order to compensate for the low molar absorption coefficient, it is effective to increase the absorbance by accumulating the dye at high concentration in the particles to gain molarity of the dye. On the other hand, in the conventional technique, the accumulation of the dye in the particle at high concentration causes a trade-off in the quantum yield due to concentration quenching. In order to avoid concentration quenching, it is necessary to use dyes with solid-state luminescence properties.

Recently, highly luminescent aggregation-induced luminescent dye-containing particles have been reported (see Patent Document 1 and Non-Patent Document 3), but cell autofluorescence cannot be avoided, and the problems of the luminescent particles that achieve both aggregation-induced luminescence and delayed luminescence disclosed in Non-Patent Document 1 are as described above. Therefore, the conventional technology has problems in high-sensitivity imaging applications.

### PRIOR ART DOCUMENTS

### Patent Document

Patent Document 1: JP-A 2016-199751

### Non-Patent Documents

Non-Patent Document 1: T. Li, et al., Adv. Sci. 2017, 4, 1600166
Non-Patent Document 2: Y. Tsuchiya, et al., Chem. Comm. 2019, 55, 5215
Non-Patent Document 3: K. Li, et al., Scientific Reports 3, Article number: 1150 (2013)

### SUMMARY OF THE INVENTION

### Problem to be Solved by the Invention

The present invention was made in view of the above problems and circumstances, and the problem to be solved is to provide luminescent nanoparticles and luminescent labeling materials for pathological diagnosis for enabling high sensitivity imaging that can avoid the adverse effects of cell autofluorescence on bioimaging.

### Means to Solve the Problems

In order to solve the above problem, the present inventor investigated the cause of the above problem, and as a result, the inventor discovered the luminescent nanoparticles for enabling high-sensitivity imaging that can avoid the adverse effects of cell autofluorescence by making the linking group connecting the electron-withdrawing group and the electron-donating group of the dye contained in the luminescent nanoparticle a backbone containing 2 to 4 atoms. The luminescent nanoparticles were found to enable high-sensitivity imaging that avoids the adverse effects of cell autofluorescence.

In other words, the above problem for the present invention is solved by the following means.

1. Luminescent nanoparticles containing a luminescent dye, wherein the luminescent dye is a compound having a structure represented by the following Formula (1), and the luminescent nanoparticles have at least one of delayed luminescence or long Stokes shift luminescence.

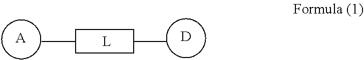

In the formula, A represents an electron-withdrawing group; D represents an electron-donating group; L represents a linking group having a backbone containing 2 to 4 atoms; the 2 to 4 atoms are a carbon atom, an oxygen atom, a nitrogen atom, a silicon atom, a sulfur atom, a boron atom, or a phosphorus atom; and the linking group may have a hydrogen atom or a substituent.

2. The luminescent nanoparticle according to item 1, wherein Formula (1) is represented by at least one of the following Formulas (2) to (6),

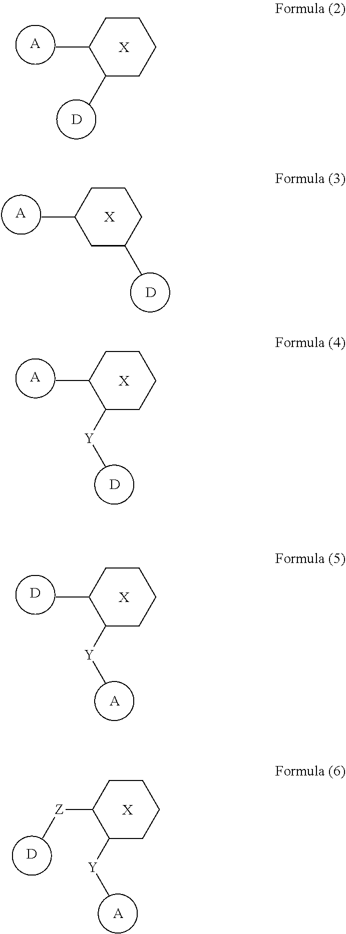

In the formulas, A represents an electron-withdrawing group; D represents an electron-donating group; X represents a cyclic saturated hydrocarbon group, a cyclic unsaturated hydrocarbon group, an aryl group or a heteroaryl group; Y and Z represent a carbon atom, an oxygen atom, a nitrogen atom, a silicon atom, a sulfur atom, a boron atom, or a phosphorus atom and may have a hydrogen atom or a substituent; and Y in Formulas (4) and (5) may be a linking group having a backbone containing two atoms.

3. The luminescent nanoparticles according to item 1 or 2, having delayed luminescence.
4. The luminescent nanoparticles according to any one of items 1 to 3, having intramolecular exciplex luminescence.
5. The luminescent nanoparticles according to any one of items 1 to 4, having long Stokes shift luminescence.
6. The luminescent nanoparticles according to any one of items 2 to 5, wherein in Formulas (2) to (6), A represents “a carbonyl group which may be substituted”, “a sulfonyl group which may be substituted”, “a boryl group which may be substituted”, “a phosphine oxide group which may be substituted”, “an aryl group which may be substituted with an electron-withdrawing group”, “an electron-donating heterocyclic group substituted with a cyano group, a fluorine atom, an alkyl group substituted with a fluorine atom, or a nitrogen-containing aromatic six-membered ring-containing heterocyclic group”, or “an electron-withdrawing heterocyclic group which may be substituted”; D represents “an aryl group substituted with an electron-donating group”, “an electron-donating heterocyclic group which may be substituted with a substituent other than a cyano group, a fluorine atom, an alkyl group substituted with a fluorine atom, or a nitrogen-containing aromatic six-membered ring-containing heterocyclic group”, or “an amino group which may be substituted”; and X represents an aryl group or a heteroaryl group.
7. The luminescent nanoparticles according to any one of items 2 to 5, wherein in Formulas (4) to (6), A represents “a carbonyl group which may be substituted”, “a sulfonyl group which may be substituted”, “a boryl group which may be substituted”, “a phosphine oxide group which may be substituted”, “an aryl group which may be substituted with an electron-withdrawing group”, “an electron-donating heterocyclic group substituted with a cyano group, a fluorine atom, an alkyl group substituted with a fluorine atom, or a nitrogen-containing aromatic six-membered ring-containing heterocyclic group”, or “an electron-withdrawing heterocyclic group which may be substituted”; D represents “an aryl group substituted with an electron-donating group”, “an electron-donating heterocyclic group which may be substituted with a substituent other than a cyano group, a fluorine atom, an alkyl group substituted with a fluorine atom, or a nitrogen-containing aromatic six-membered ring-containing heterocyclic group”, or “an amino group which may be substituted”; X represents an aryl group or a heteroaryl group; and Y represents a carbon atom, an oxygen atom, or a silicon atom.
8. The luminescent nanoparticles according to any one of items 1 to 7, wherein the luminescent dye has a hydrophilic group.
9. The luminescent nanoparticles according to any one of items 1 to 8, containing a binder.
10. The luminescent nanoparticles according to any one of item 9, wherein the binder present on a surface of the luminescent nanoparticle among the binder has a hydrophilic group.
11. The luminescent nanoparticles according to item 9 or 10, wherein the binder and the luminescent dye form a covalent bond.
12. A luminescent labeling material for pathological diagnosis, wherein a target-directed ligand is covalently bonded to a surface of the luminescent nanoparticle according to any one of items 1 to 11.

### Effects of the Invention

By the above means of the present invention, it is possible to provide luminescent nanoparticles and luminescent labeling materials for pathological diagnosis for enabling high-sensitivity imaging that can avoid the adverse effects of cell autofluorescence on bioimaging.

The expression mechanism or action mechanism of the effect of the present invention is not clear but is inferred as follows.

An electron-donating group and an electron-withdrawing group are connected by a linking group having a backbone containing 2 to 4 atoms to effectively transfer electrons between the electron-donating group and the electron-withdrawing group in an excited state. By causing the electron-donating group and the electron-withdrawing group to have a twisted structure, heat-activated delayed fluorescence is generated, or by connecting the electron-donating group and the electron-withdrawing group with a non-conjugated atom, it is possible to emit intramolecular exciplex luminescence with delayed luminescence with high luminescence. Furthermore, for the same reason, luminescence having a long Stokes shift may also be emitted with high luminescence.

Luminescence in the solid state (molecular aggregation state), i.e., “solid-state luminescence”, is also achieved by connecting an electron-donating group and an electron-withdrawing group with a backbone containing 2 to 4 atoms and maintaining the distance between the electron-donating group and the electron-withdrawing group in a close state. The reason for this is that maintaining the distance between the electron-donating and electron-withdrawing groups in a close state enables rapid electronic transitions and the formation of highly stable excited states when excited and avoids the hindrance of quenching due to intermolecular interactions. As a result, it is inferred that concentration quenching may be avoided and solid-state luminescence may be achieved.

Therefore, it is considered possible to provide luminescent nanoparticles for enabling high-sensitivity imaging that can avoid adverse effects of cell autofluorescence on bioimaging.

### Embodiments to Carry Out the Invention

The luminescent nanoparticle of the present invention is a luminescent nanoparticle containing a luminescent dye, wherein the luminescent dye is a compound represented by Formula (1) and is characterized by having at least one of delayed luminescence or long Stokes shift luminescence.

This feature is a technical feature common to or corresponding to the following embodiments.

As an embodiment of the present invention, it is preferred that Formula (1) is represented by at least one of Formulas (2) to (6) from the viewpoint of expression of the effect of the present invention.

It is preferred that the luminescent nanoparticles of the present invention have delayed luminescence from the viewpoint of reducing the effect of cell autofluorescence. It is further preferred that they have intramolecular exciplex luminescence properties that emit delayed luminescence with high luminescence intensity. Furthermore, it is also preferred to have a long Stokes shift luminescence property.

As an embodiment of the present invention, from the viewpoint of expressing the effect of the present invention, it is preferred that A, D and X in Formulas (2) to (6) represent the substituents or atoms exemplified above.

As an embodiment of the present invention, from the viewpoint of expressing the effect of the present invention, it is preferred that A, D, X and Y in Formulas (4) to (6) represent the substituents or atoms as exemplified above.

It is preferred that in the luminescent nanoparticles of the present invention, the luminescent dye has a hydrophilic group. The inclusion of a binder is also preferred in that it enables the particles to have a special function on their surface via the binder. It is also preferred in that the dye may be easily incorporated into the binder particles in a dispersed state, thereby enhancing the luminescence in the particles.

Furthermore, it is preferred that the binder on the surface of the luminescent nanoparticles among the binder has a hydrophilic group in order to inhibit aggregation of the particles.

As an embodiment, it is preferred that the binder and the luminescent dye form a covalent bond to prevent leakage of the luminescent dye from the particles.

The luminescent labeling material for pathological diagnosis is characterized in that a target-directed ligand is covalently bonded to the surface of the above luminescent nanoparticle.

A detailed description of the present invention, its components, and the form and embodiment of carrying out the present invention will be given below. In this application, “to” is used in the sense of including the numerical values described before and after “to” as lower and upper limits.

### 1. Outline of Luminescent Nanoparticle of the Present Invention

The luminescent nanoparticle of the present invention is a luminescent nanoparticle containing a luminescent dye, wherein the luminescent dye is a compound having a structure represented by Formula (1) and is characterized by having at least one of delayed luminescence or long Stokes shift luminescence.

Each component will be described in turn below.

### <1.1> Shape of Luminescent Nanoparticles

The term “luminescent nanoparticles” means particles containing a luminescent dye and having an average particle size in the range of 1 to 1000 nm. The average particle size is preferably in the range of 30 to 500 nm, and more preferably in the range of 5 to 200 nm.

The average particle size of the produced luminescent nanoparticles may be measured by methods known in the art. Specifically, an electron micrograph is taken at an appropriate magnification using a scanning electron microscope (SEM), the cross-sectional area of the luminescent nanoparticles is measured, and the measured value is used as the area of a circle corresponding to the diameter (equivalent circle diameter).

The mean particle size (average particle size) and coefficient of variation of a population of luminescent nanoparticles are calculated by measuring the particle size (particle size) of a sufficient number (e.g., 1000) of luminescent nanoparticles as described above, then the average particle size is calculated as an arithmetic mean thereof, and the coefficient of variation is calculated by the formula: 100×standard deviation of particle size/average particle size.

In the present invention, the coefficient of variation, which indicates the variation in particle size, is not particularly limited, but it is usually 20% or less, preferably 5 to 15%.

### <1.2> Luminescence Characteristics of Luminescent Nanoparticles

The luminescent nanoparticle of the present invention is a luminescent nanoparticle containing a luminescent dye, wherein the luminescent dye is a compound having a structure represented by Formula (1) and is characterized by having at least one of delayed luminescence or long Stokes shift luminescence.

The luminescent nanoparticles of the present invention basically emit heat-activated delayed fluorescent luminescence or exciplex luminescence among delayed luminescence but have the following luminescence properties.

Here, “fluorescence” refers to the light emitted when an electron transitions from the excited singlet state to the ground singlet state (allowed transition).

In contrast, “phosphorescence” refers to the light emitted when an electron transitions from the excited triplet state to the ground singlet state (forbidden transition).

### <Delayed Luminescence>

It is preferred that the luminescent nanoparticles of the present invention have delayed luminescence properties in that the adverse effects of cell autofluorescence may be avoided. The delayed luminescence property enables luminescence imaging at a time after the disappearance of the autofluorescence of the cell.

The term “delayed luminescence” refers to the phenomenon where, for example, when intersystem crossing occurs from the lowest singlet excited state (S₁) to the energetically lower excited triplet state (T₁), if the energy difference between S₁-T₁ is small, the two states are in a thermal equilibrium state, and long-lived fluorescence is emitted in the same manner as phosphorescence from the T₁ state. It also refers to a phenomenon in which energy is once held in a metastable state such as the charge-separated state, and then the energy released by charge recombination is emitted as light with a long lifetime.

For example, the emission lifetime (decay time) of normal fluorescence is very short, less than nanoseconds, but delayed fluorescence may extend the emission lifetime to the order of minutes because it undergoes a metastable state.

Phosphorescence, heat-activated delayed fluorescence, and exciplex luminescence are examples of luminescence methods that cause delayed luminescence. Since phosphorescence is light emission due to forbidden transition, phosphorescence is less likely to occur than light emission due to allowed transition, and thus phosphorescence tends to exhibit delayed luminescence.

### (Heat-Activated Delayed Fluorescence)

The term “heat-activated delayed fluorescence” refers to light emitted by singlet excitons generated by transition of triplet excitons due to reverse intersystem crossing caused by thermal energy. Since reverse intersystem crossing is involved, the heat-activated delayed fluorescence tends to exhibit delayed luminescence. For the reverse intersystem crossing to occur, the absolute value (ΔEₛₜ) of the difference between the lowest excited singlet energy level (S₁) and the lowest excited triplet energy level (T₁) must be extremely small.

### (Exciplex Luminescence)

The term “exciplex luminescence” refers to light emitted when an exciplex (excited complex) returns to its ground state. Here, the term “exciplex” is an ABₙ* formed by a chemical species A* in the excited electronic state with n chemical species B in the ground state. Exciplex is known to have an extremely small ΔEₛₜ, and is likely to cause reverse intersystem crossing, so it tends to exhibit delayed luminescence.

Heat-activated delayed fluorescence involves the process of immediate emission from S₁ without undergoing reverse intersystem crossing, whereas exciplex emission is emission from the exciplex level only. Therefore, the intensity of the delayed luminescence component is greater in exciplex luminescence than in heat-activated delayed fluorescence. Furthermore, the luminescent dyes contained in the luminescent nanoparticles of the present invention have a structure that can form an exciplex intramolecularly, allowing them to form an exciplex that is stronger than the common intermolecular exciplex.

### <Long Stokes Shift Luminescence>

It is preferred that the luminescent nanoparticles of the present invention have long Stokes shift luminescence properties in that the adverse effects of cell autofluorescence can be avoided. The long Stokes shift luminescence enables luminescence imaging in a wavelength range different from the wavelength range where the emission intensity of the cell autofluorescence is strong.

The term “Long Stokes shift luminescence” refers to photoluminescence in which the emission (maximum) wavelength shifts to a longer wavelength than the optical absorption (maximum) wavelength, and the difference between the two maximum wavelengths is larger than usual. In the present invention, it refers to a luminescence in which the difference between the optical absorption wavelength and the emission wavelength is 100 nm or more. The heat-activated delayed fluorescence and exciplex luminescence described above are known as luminescence schemes that tend to exhibit long Stokes shift luminescence properties.

It is more preferred that the difference between the optical absorption wavelength and the emission wavelength is 150 nm or more, and 200 nm or more is even more preferred.

### <1.3> Structure of Luminescent Dye

The luminescent dye used in the present invention is preferably a compound having a structure represented by the following Formula (1), and also a compound having a structure represented by the following Formulas (2) to (6).

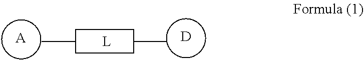

In the formula, A represents an electron-withdrawing group; D represents an electron-donating group; L represents a linking group having a backbone containing 2 to 4 atoms; the 2 to 4 atoms are a carbon atom, an oxygen atom, a nitrogen atom, a silicon atom, a sulfur atom, a boron atom, or a phosphorus atom; and the linking group may have a hydrogen atom or a substituent.

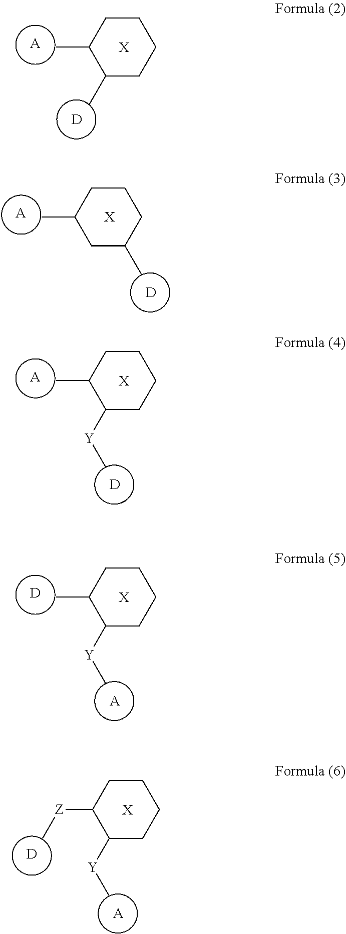

In the formulas, A represents an electron-withdrawing group; D represents an electron-donating group; X represents a cyclic saturated hydrocarbon group, a cyclic unsaturated hydrocarbon group, an aryl group or a heteroaryl group; Y and Z represent a carbon atom, an oxygen atom, a nitrogen atom, a silicon atom, a sulfur atom, a boron atom, or a phosphorus atom and may have a hydrogen atom or a substituent. Y in Formulas (4) and (5) may be a linking group having a backbone containing two atoms.

### (Electron-Withdrawing Group: A)

In Formulas (1) to (6), the electron-withdrawing group represented by A is preferably “a carbonyl group which may be substituted”, “a sulfonyl group which may be substituted”, “a boryl group which may be substituted”, “a phosphine oxide group which may be substituted”, “an aryl group which may be substituted with an electron-withdrawing group”, “an electron-donating heterocyclic group substituted with a cyano group, a fluorine atom, an alkyl group substituted with a fluorine atom, or a nitrogen-containing aromatic six-membered ring-containing heterocyclic group”, or “an electron-withdrawing heterocyclic group which may be substituted”.

The alkyl group of the “alkyl group substituted with a fluorine atom” represented by A is the same as described below, and the same is preferred.

The aryl group of the “aryl group which may be substituted” represented by A is the same as described below, and the same is preferred.

As the electron-withdrawing heterocyclic group of the “electron-withdrawing heterocyclic group which may be substituted” represented by A, a group derived from an electron-withdrawing heterocyclic ring having 3 to 24 carbons is preferred. Examples of such a heterocyclic ring include a dibenzothiophene oxide ring, a dibenzothiophene dioxide ring, a pyridine ring, a pyridazine ring, a pyrimidine ring, a pyrazine ring, a triazine ring, a quinoline ring, an isoquinoline ring, a quinazoline ring, a quinoline ring, a quinoxaline ring, a phthalazine ring, a pteridine ring, a phenanthridine ring, a phenanthroline ring, an azacarbazole ring, a diazacarbazole ring, a dibenzofuran ring, a dibenzosilole ring, an azadibenzofuran ring, a diazadibenzofuran ring, a dibenzoborol ring, a dibenzophosphoroxide ring, a quinone ring, a tetracyanoquinodimethane ring, a quasi-planar or plane-fixed triarylborane ring, and a quasi-planar or plane-fixed triarylphosphine ring. More preferable examples include a pyridine ring, a pyrimidine ring, a pyrazine ring, a triazine ring, a quinoline ring, an isoquinoline ring, a quinazoline ring, a quinoxaline ring, a phenanthridine ring, a phenanthroline ring, a dibenzofuran ring, an azadibenzofuran ring, a diazadibenzofuran ring, a coumarin ring, a p-benzoquinone ring, a tetracyanoquinodimethane ring, a triarylborane ring fixed to a quasi-plane or plane by a monoatomic bridge or a single bond, a triarylphosphorane ring fixed to a quasi-plane or plane by a monoatomic bridge or a single bond. More preferable example is a nitrogen-containing aromatic heterocyclic ring. Particularly preferable examples are a nitrogen-containing aromatic six-membered ring, a dibenzofuran ring, an azadibenzofuran ring, a coumarin ring, a diazadibenzofuran ring, a triarylborane ring fixed to a quasi-plane or plane bridged with an oxygen atom, a nitrogen atom, a sulfur atom or a carbon atom, and a triarylphosphine ring fixed to a quasi-plane or plane bridged with an oxygen atom, a nitrogen atom, a sulfur atom or a carbon atom. The electron-withdrawing heterocyclic ring may be formed by bonding two or more of the same or different above heterocyclic rings.

Examples of the substituent of the “electron-withdrawing heterocyclic group which may be substituted” represented by A include a deuterium atom, a fluorine atom, a cyano group, an alkyl group which may be substituted with a fluorine atom, an aryl group which may be substituted with an alkyl group which may be substituted with a fluorine atom, an aryl group which may be substituted with a fluorine atom, an aryl group which may be substituted with a cyano group. Specific examples of the alkyl group and aryl group are the same as above, and the same is preferred. Electron-donating substituents may also be substituted as long as the electron-withdrawing property of the electron-withdrawing heterocyclic group is not impaired, examples of which include a carbazole group.

Examples of the electron-withdrawing group of the “aryl group which may be substituted with an electron-withdrawing group” represented by A includes a fluorine atom, a cyano group, an alkyl group substituted with a fluorine atom, a carbonyl group which may be substituted, a sulfonyl group which may be substituted, a phosphine oxide group, a boryl group which may be substituted, an electron-withdrawing heterocyclic group which may be substituted. More preferable examples are a fluorine atom, a cyano group, an alkyl group substituted with a fluorine atom and an electron-withdrawing heterocyclic group which may be substituted.

The “electron-donating heterocyclic group” of the “electron-donating heterocyclic group substituted with a cyano group, a fluorine atom, an alkyl group substituted with a fluorine atom, or a nitrogen-containing aromatic six-membered heterocyclic group” represented by A is the same as described below and the same is preferred.

The “nitrogen-containing aromatic six-membered ring-containing heterocycle” of the “electron-donating heterocyclic group substituted with a cyano group, a fluorine atom, an alkyl group substituted with a fluorine atom, or a nitrogen-containing aromatic six-membered ring-containing heterocyclic group” represented by A means a group derived from a heterocyclic ring containing a nitrogen-containing aromatic six-membered ring. Examples of the heterocyclic ring containing a nitrogen-containing aromatic six-membered ring include a pyridine ring, a pyridazine ring, a pyrimidine ring, a pyrazine ring, a triazine ring, a quinoline ring, an isoquinoline ring, a quinazoline ring, a quinoline ring, a quinoxaline ring, a quinoline ring, a phthalazine ring, a pteridine ring, a phenanthridine ring, a phenanthroline ring, an azacarbazole ring, a diazacarbazole ring, an azadibenzofuran ring, and a diazadibenzofuran ring. More preferable examples include a pyridine ring, a pyrimidine ring, a triazine ring, an azacarbazole ring, a diazacarbazole ring, an azadibenzofuran ring and a diazadibenzofuran ring.

Examples of the substituent of the “carbonyl group which may be substituted”, “a sulfonyl group which may be substituted”, “a boryl group which may be substituted” and “a phosphine oxide group which may be substituted” represented by A include a fluorine atom, cyano group, fluorine atom, an alkyl group which may be substituted with a fluorine atom, an aryl group which may be substituted, and an electron-withdrawing heterocyclic group which may be substituted. Specific examples include the same as described above, and the same is preferred.

Since the electron-withdrawing group A easily interacts with another electron-withdrawing group A or electron-donating group D, among these, the electron-withdrawing group A is preferred to be “an aryl group which may be substituted with an electron-withdrawing group”, “an electron-donating heterocyclic group which may be substituted with a cyano group, a fluorine atom, an alkyl group substituted with a fluorine atom, or a nitrogen-containing aromatic six-membered ring-containing heterocyclic group”, or “an electron-withdrawing heterocyclic group which may be substituted”. This is because these groups are highly planar and thus tend to spatially overlap and interact with other electron-withdrawing groups A or electron-donating groups D.

The “aryl group which may be substituted with an electron-withdrawing group”, “electron-donating heterocyclic group substituted with a cyano group, a fluorine atom, an alkyl group substituted with a fluorine atom, or a nitrogen-containing aromatic six-membered ring-containing heterocyclic group” or “electron-withdrawing heterocyclic group which may be substituted” is selected from the following in terms of having high electron-withdrawing property. It is preferably an aryl group substituted with a cyano group, an electron-withdrawing heterocyclic group which may be substituted with a cyano group, an unsubstituted nitrogen-containing aromatic six-membered ring group, a nitrogen-containing aromatic six-membered ring group substituted with a fluorine atom, a nitrogen-containing aromatic six-membered ring group substituted with a cyano group, an electron-donating heterocyclic group substituted with a cyano group, an electron-donating heterocyclic group substituted with an azacarbazole, an electron-donating heterocyclic group substituted with an azadibenzofuran, a nitrogen-containing aromatic six-membered ring group substituted with a fluorine substituted alkyl group, or a nitrogen-containing aromatic six-membered ring group substituted with an aryl group which may be substituted. More preferably, it is a phenyl group substituted with a cyano group, a carbazolyl group substituted with a cyano group, a carbazolyl group substituted with an azacarbazole, a carbazolyl group substituted with an azadibenzofuran, a dibenzofuryl group substituted with a cyano group, an azadibenzofuryl group, a diazadibenzofuryl group, a pyridyl group substituted with a cyano group, a pyrazyl group which may be substituted, a pyrimidyl group which may be substituted, or a triazyl group which may be substituted. Still more preferably, it is a cyanophenyl group, a m-dicyanophenyl group, a 3-cyanocarbazolyl group, a 3-(9-azacarbazolyl)carbazolyl group, a 3-azadibenzofurylcarbazolyl group, a 9-azadibenzofurylcarbazolyl group, a 6-cyanocarbazolyl group, a 2-cyanodibenzofuryl group, an azadibenzofuryl group, a diazadibenzofuryl group, a 2-cyanopyridyl group, a 3-cyanopyridyl group, a 2,6-dicyanopyridyl group, a 2-cyanopyrazyl group, a 2-cyanopyrimidyl group, a 5-cyanopyrimidyl group, a 2,4-diphenylpyrimidyl group which may be substituted, a diphenyltriazyl group which may be substituted, a triphenyltriazyl group which may be substituted, a p-quinone group, a tetracyanoquinodimethane group, a tetracyanoquinodimethane group substituted with a fluorine atom, a triarylborane group fixed to a quasi-plane or plane by a monoatomic bridge or a single bond, or a triarylphosphine group fixed to a quasi-planar or planar surface by a monoatomic bridge or a single bond.

### (Electron-Donating Group: D)

In Formulas (1) to (6), the electron-donating group represented by D is preferably “an aryl group substituted with an electron-donating group”, “an electron-donating heterocyclic group which may be substituted with a substituent other than a cyano group, a fluorine atom, an alkyl group substituted with a fluorine atom, or a nitrogen-containing aromatic six-membered ring-containing heterocyclic group”, or “an amino group which may be substituted”.

As the aryl group of the “aryl group substituted with an electron-donating group” represented by D, it is preferable to be a group derived from an aromatic hydrocarbon ring having a carbon number of 6 to 24 is preferred. Examples of such aromatic hydrocarbon rings include a benzene ring, an indene ring, a naphthalene ring, an azulene ring, a fluorene ring, a phenanthrene ring, an anthracene ring, an acenaphthylene ring, a biphenylene ring, a naphthacene ring, a pyrene ring, a pentalene ring, an aceanthrylene ring, a heptalene ring, a triphenylene ring, an as-indacene ring, a chrysene ring, an s-indacene ring, a pleiadene ring, a phenalene ring, a fluoranthene ring, a perylene ring, an acephenanthrylene ring, a biphenyl ring, a terphenyl ring, and a tetraphenyl ring. More preferably, they are a benzene ring, a naphthalene ring, a fluorene ring, a phenanthrene ring, an anthracene ring, a biphenylene ring, a chrysene ring, a pyrene ring, a triphenylene ring, a chrysene ring, a fluoranthene ring, a perylene ring, a biphenyl ring, and a terphenyl ring.

As the “electron-donating heterocyclic group” of the “electron-donating heterocyclic group which may be substituted with substituents other than a cyano group, a fluorine atom, an alkyl group substituted with a fluorine atom, and a nitrogen-containing aromatic six-membered ring-containing heterocyclic group” represented by D, preferred examples thereof are groups derived from an electron-donating heterocyclic ring having a carbon number of 4 to 24. Examples of such heterocyclic rings include a pyrrole ring, an indole ring, a carbazole ring, an indoloindole ring, a 9,10-dihydroacridine ring, a 10,11-dihydrodibenzoazepine ring, a 5,10-dihydrodibenzoazacillin ring, a 5,10-dihydrodiphenazine ring, a phenoxazine ring, a phenothiazine ring, a dibenzothiophene ring, a benzofurylindole ring, a benzothienoindole ring, an indolocarbazole ring, a benzofurylcarbazole ring, a benzothienocarbazole ring, a benzothienobenzothiophene ring, a benzocarbazole ring, a dibenzocarbazole ring, a tetrathiafulvalene ring, a thianthrene, and a triarylamine ring fixed to a quasi-plane or plane. More preferably, they are a carbazole ring, an indoloindole ring, a 9,10-dihydroacridine ring, a phenoxazine ring, a phenothiazine ring, a dibenzothiophene ring, a benzofurylindole ring, a benzocarbazole ring, a dibenzofuran ring, a triarylamine ring fixed to a quasi-plane or plane by a monoatomic bridge of an oxygen atom, a nitrogen atom, a sulfur atom or a carbon atom, or a single bond. The electron-donating heterocyclic ring may be a combination of two or more of the same or different heterocyclic rings described above. The dibenzofuran ring may function as an electron-donating group D as a whole when substituted with an electron-donating substituent (e.g., a carbazole group), but may function as an electron-withdrawing group A when unsubstituted or substituted with an electron-drawing substituent.

The “electron-donating group” of the “aryl group substituted with an electron-donating group” represented by D includes an alkyl group which may be substituted, an alkoxy group which may be substituted, an amino group which may be substituted, and an electron-donating heterocyclic group which may be substituted. Preferable examples thereof include an amino group which may be substituted and an electron-donating heterocyclic group which may be substituted.

The alkyl group as the “electron-donating group” of the “aryl group substituted with an electron-donating group” represented by D may be a linear, branched, or cyclic alkyl group having a carbon number of 1 to 20. Examples thereof include a methyl group, an ethyl group, an n-propyl group, an isopropyl group, an n-butyl group, a t-butyl group, an n-pentyl group, a neopentyl group, an n-hexyl group, a cyclohexyl group, a 2-ethylhexyl group, an n-heptyl group, an n-octyl group, a 2-hexyloctyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-icosyl group, and an adamantyl group. Preferable examples thereof include a methyl group, an ethyl group, an isopropyl group, a t-butyl group, a cyclohexyl group, a 2-ethylhexyl group, a 2-hexyloctyl group, and an adamantyl group.

The alkoxy group of the “electron-donating group” of the “aryl group substituted with an electron-donating group” represented by D may be a linear, branched, or cyclic alkoxy group having a carbon number of 1 to 20. Specific examples thereof include a methoxy group, an ethoxy group, an n-propoxy group, an isopropoxy group, n-an butoxy group, an isobutoxy group, a t-butoxy group, an n-pentyloxy group, a neopentyloxy group, an n-hexyloxy group, a cyclohexyloxy group, an n-heptyloxy group, an n-heptyloxy group, an n-octyloxy group, a 2-ethylhexyloxy group, a nonyloxy group, a decyloxy group, a 3,7-dimethyloctyloxy group, an n-undecyloxy group, an n-dodecyloxy group, an n-tridecyloxy group, an n-tetradecyloxy group, a 2-n-hexyl-n-octyloxy group, an n-pentadecyloxy group, an n-hexadecyloxy group, an n-heptadecyloxy group, an n-octadecyloxy group, an n-nonadecyloxy group, and an n-icosyloxy group. Preferable examples include a methoxy group, an ethoxy group, an isopropoxy group, a t-butoxy group, a cyclohexyloxy group, a 2-ethylhexyloxy group, and a 2-hexyloctyloxy group.

As the substituent of the “amino group which may be substitute” of the “aryl group substituted with an electron-donating group” represented by D, the following may be cited. Examples thereof include an alkyl group which may be substitute, an aryl ring which may be substitute with an optionally substituted alkyl group, an aryl ring which may be substitute with an optionally substituted alkoxy group, and an aryl ring which may be substitute with an optionally substituted amino group. With respect to the alkyl group, the alkoxy group, the amino group, and the aryl group, the same as those mentioned above are mentioned and the same groups are preferred.

In the “electron-donating heterocyclic group which may be substituted with substituents other than a cyano group, a fluorine atom, an alkyl group substituted with a fluorine atom and a nitrogen-containing aromatic six-membered ring-containing heterocyclic group” represented by D, “the substituents other than a cyano group, a fluorine atom, an alkyl group substituted with a fluorine atom and a nitrogen-containing aromatic six-membered ring-containing heterocyclic group” means substituents other than a cyano group, a fluorine atom, an alkyl group substituted with a fluorine atom, and a nitrogen-containing aromatic six-membered ring-containing heterocyclic group described above. It is preferable that the substituent is an electron-donating group or an aryl group. Examples of the electron-donating group or aryl group include an alkyl group, an alkoxy group, an aryl group which may be substituted with an alkyl group, an aryl ring which may be substituted with an alky group, an aryl ring which may be substituted with an alkoxy group which may be substituted, an aryl ring which may be substituted with an amino group which may be substituted, and an amino group which may be substituted. Specific examples thereof include the same as those described above, and the same groups are preferred. In addition, it may be an electron-withdrawing substituent as long as it does not impair the electron-donating property of the electron-donating heterocyclic group, and examples thereof include a dibenzofuryl group.

The “electron-donating heterocyclic group which may be substituted” of the “aryl group substituted with an electron-donating group” represented by D is the same as described above, and the same is preferred.

The substituents of the “amino group which may be substituted” represented by D are the same as those of the “the amino group which may be substituted” above, and the same is preferred.

Among these, the electron-donating group D is preferably “an aryl group substituted with an electron-donating group” or “an electron-donating heterocyclic group which may be substituted” from the viewpoint of easy interaction with other electron-donating groups D or electron-withdrawing groups A. These groups have a high planarity, and therefore tend to overlap spatially with other electron-donating groups D or electron-withdrawing groups A and interact with them. The “aryl group substituted with an electron-donating group” or “electron-donating heterocyclic group which may be substituted” is preferably an aryl group substituted with an electron-donating group, a carbazolyl group which may be substituted, an azacarbazolyl group which may be substituted, a diazacarbazolyl group which may be substituted, a 9,10-dihydroacridyl group which may be substituted, a phenoxazyl group which may be substituted, a phenothiazyl group which may be substituted, a 5,10-dihydrophenazyl group, a tetrathiafulvalene ring, or a triarylamine ring fixed to a quasi-plane or plane bridged by an oxygen atom, a nitrogen atom, a sulfur atom, or a carbon atom.

### (Linking Group: L or X—Y)

In Formula (1), L represents a linking group having a backbone containing 2 to 4 atoms, wherein the 2 to 4 atoms are a carbon atom, an oxygen atom, a nitrogen atom, a silicon atom, a sulfur atom, a boron atom, or a phosphorus atom. The linking group may have a hydrogen atom or a substituent.

In Formulas (2) to (6), X represents a cyclic saturated hydrocarbon group, a cyclic unsaturated hydrocarbon group, an aryl group or a heteroaryl group. Y and Z represent a carbon atom, an oxygen atom, a nitrogen atom, a silicon atom, a sulfur atom, a boron atom, or a phosphorus atom and may have a hydrogen atom or substituent.

As an embodiment, from the viewpoint of expressing the effect of the present invention, it is preferred that, in Formulae (2) to (6), X represents an aryl group or a heteroaryl group. Further, in Formulas (4) to (6), it is preferred that X represents an aryl group or a heteroaryl group, and Y represents a carbon atom, an oxygen atom, or a silicon atom.

### (Hydrophilic Group)

The luminescent dye contained in the luminescent nanoparticles of the present invention is preferred to have a hydrophilic group in order to facilitate incorporation of the dye into the binder particle in a dispersed state and to enhance the luminescent property in the particles. The “hydrophilic group” refers to an atomic group that interacts strongly with water, and specific examples thereof include —OH, —SH, —COOH, —C(═O)H, —S(═O)₂OH, —S(═O)NH₂, —S(═O)₂NH₂, —P(═O)(OH)₃, —P(═O)R(OH)₂, —P(═O)R₂(OH), —P(OH)₃, —P(═O)(NH₂)₃, —P(═O)R(NH₂)₂, —P(═O)R₂(NH₂), —P(NH₂)₃, —O(C═O)OH, —NH₂, —NHR, —NHCONH₂, —NHCONHR, —NHCOOH, —Si(OH)₃, —Si(R)(OH)₂, —Si(R)₂OH, —Ge(OH)₃, —Ge(R)(OH)₂, —Ge(R)₂(OH), —Ti(OH)₃, —Ti(R)(OH)₂, —Si(NH₂)₃, —Si(R)(NH₂)₂, —B(OH)₂, —O—B(OH)₂, —O—B(OH)₂, —B(NH₂)₂, —NHB(OH)₂, and a polyethylene glycol group. The aforementioned R independently indicates a hydrogen atom or an alkyl group having a carbon number of 1 to 20. In addition, an NHS group and a maleimide group may also be mentioned as a hydrophilic group.

### (Specific Examples of Luminescent Dye)

The following are preferred specific examples of the compounds having the structure represented by Formula (1) according to the present invention, but these compounds may have further substituents, structural isomers, and the compounds are not limited only to the compounds exemplified below.

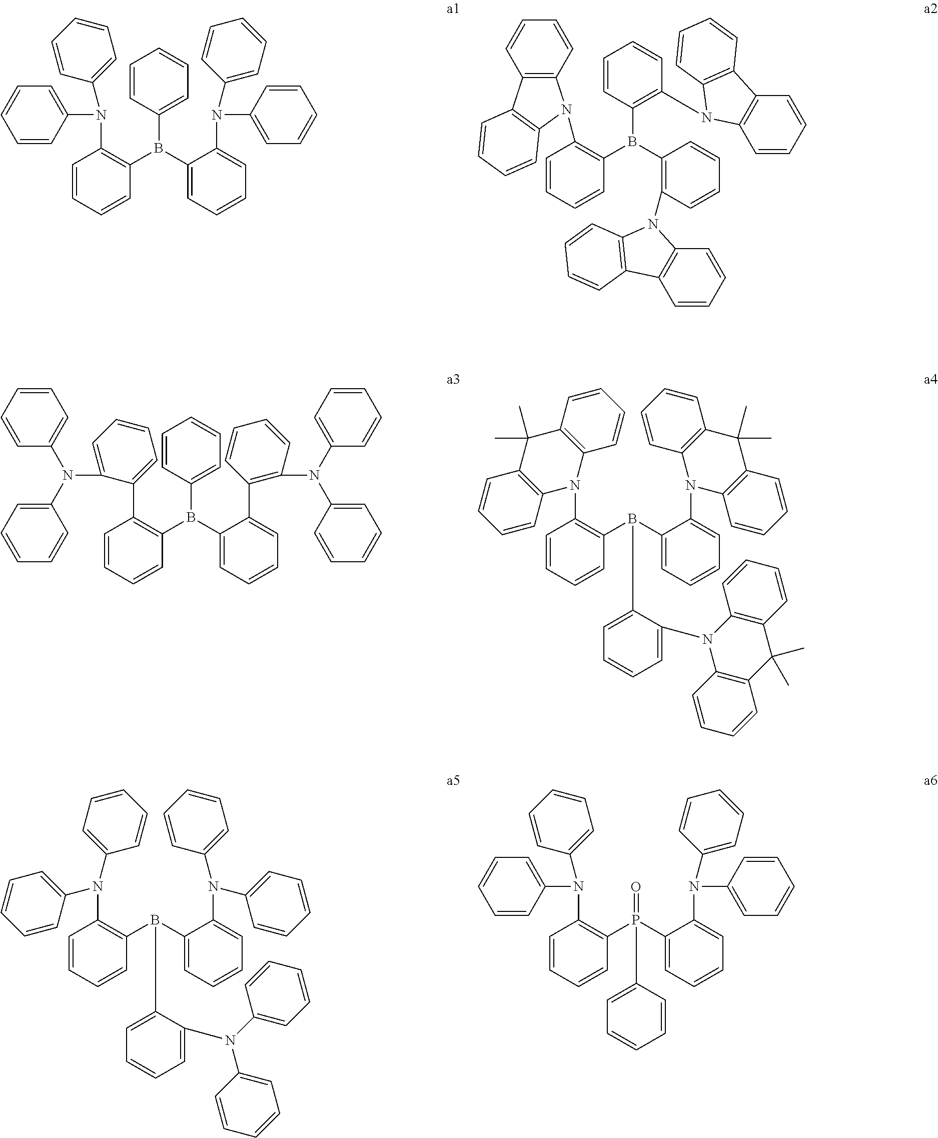

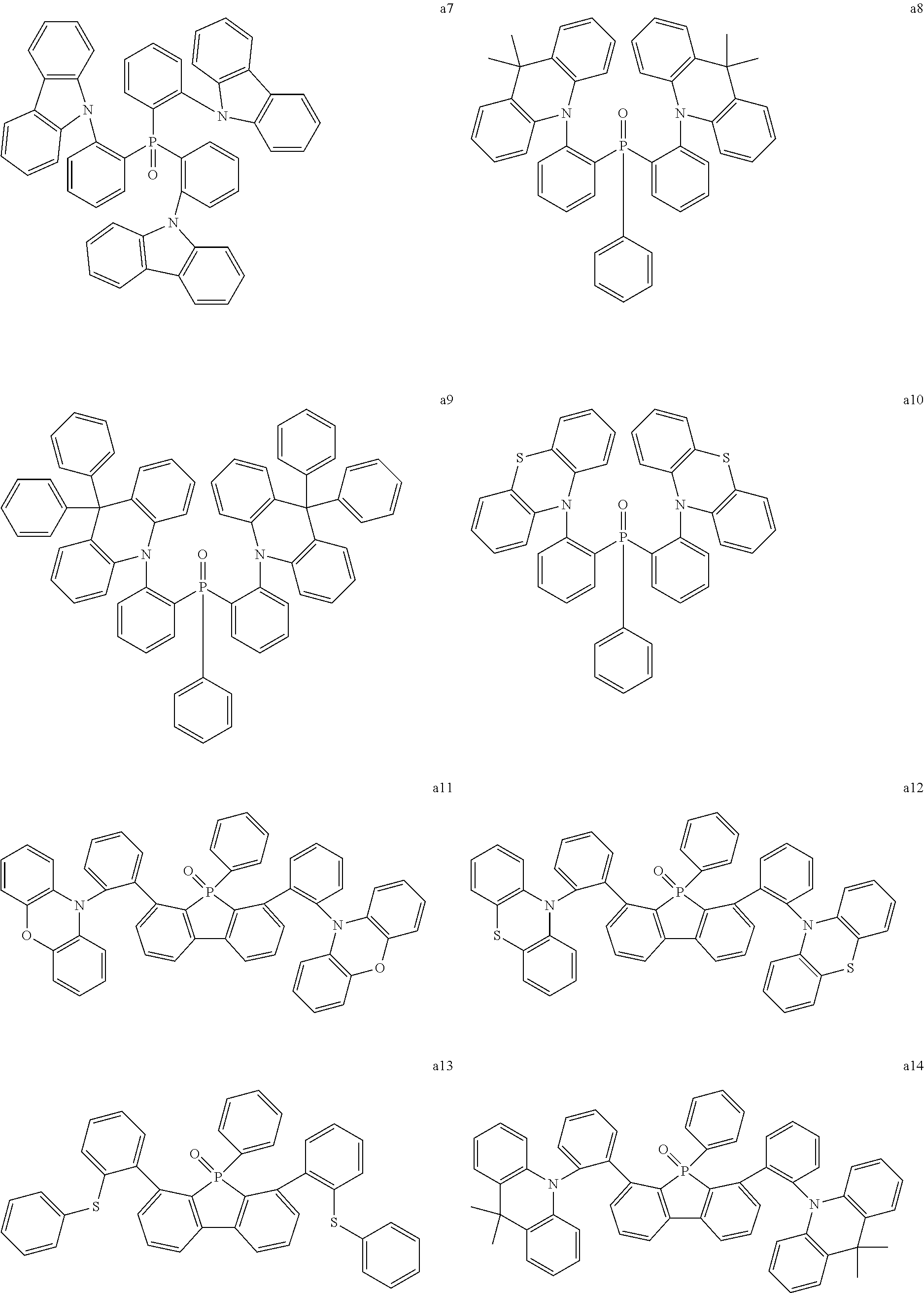

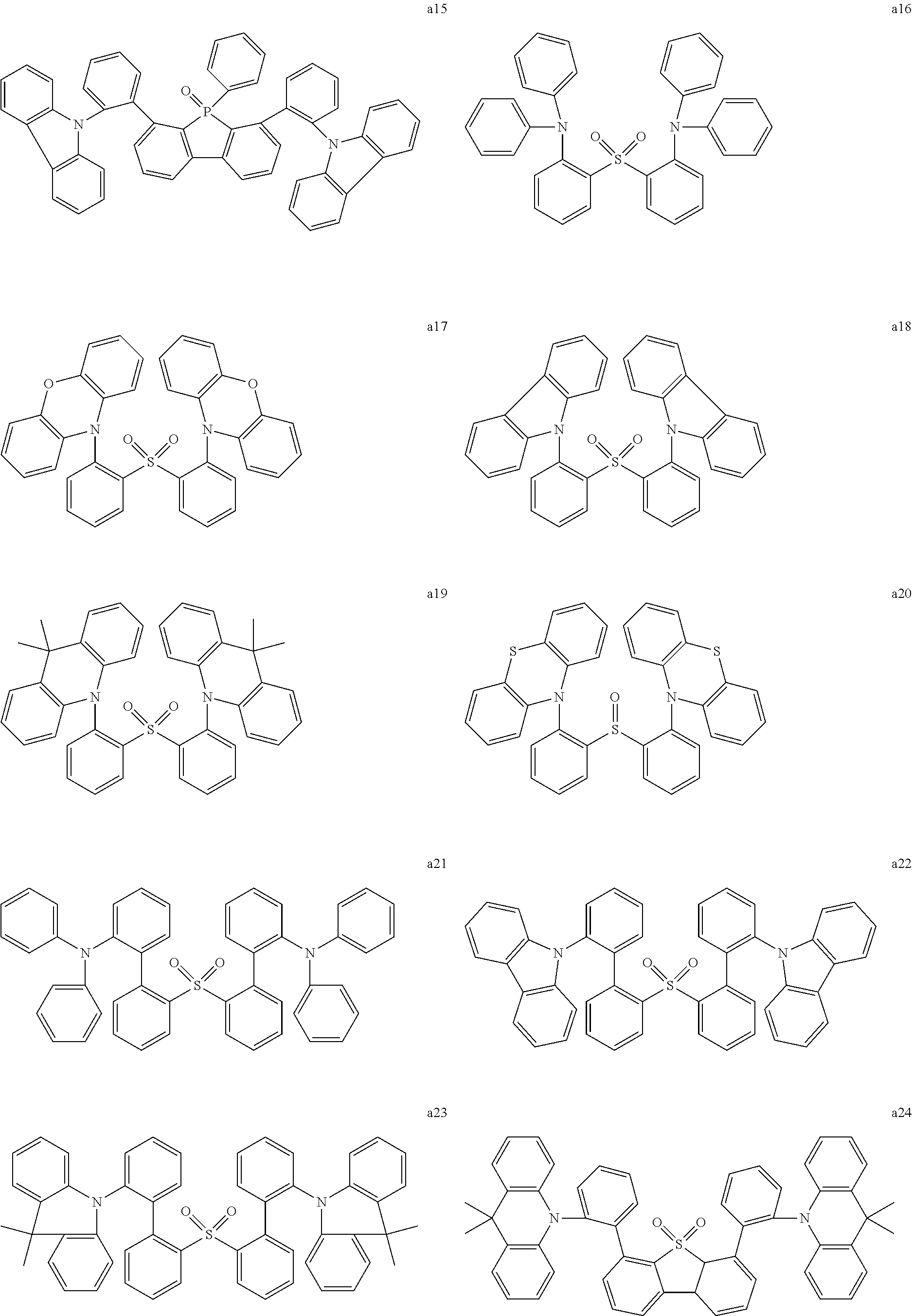

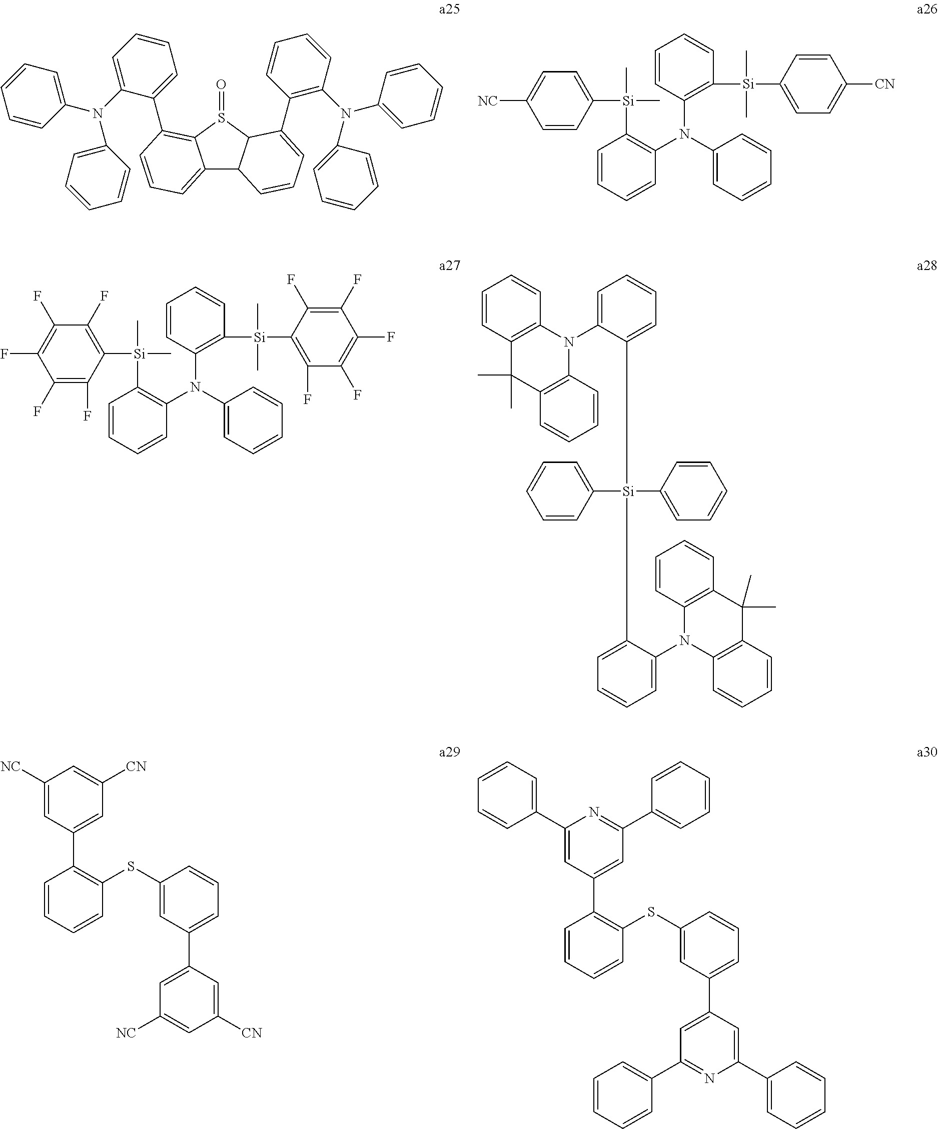

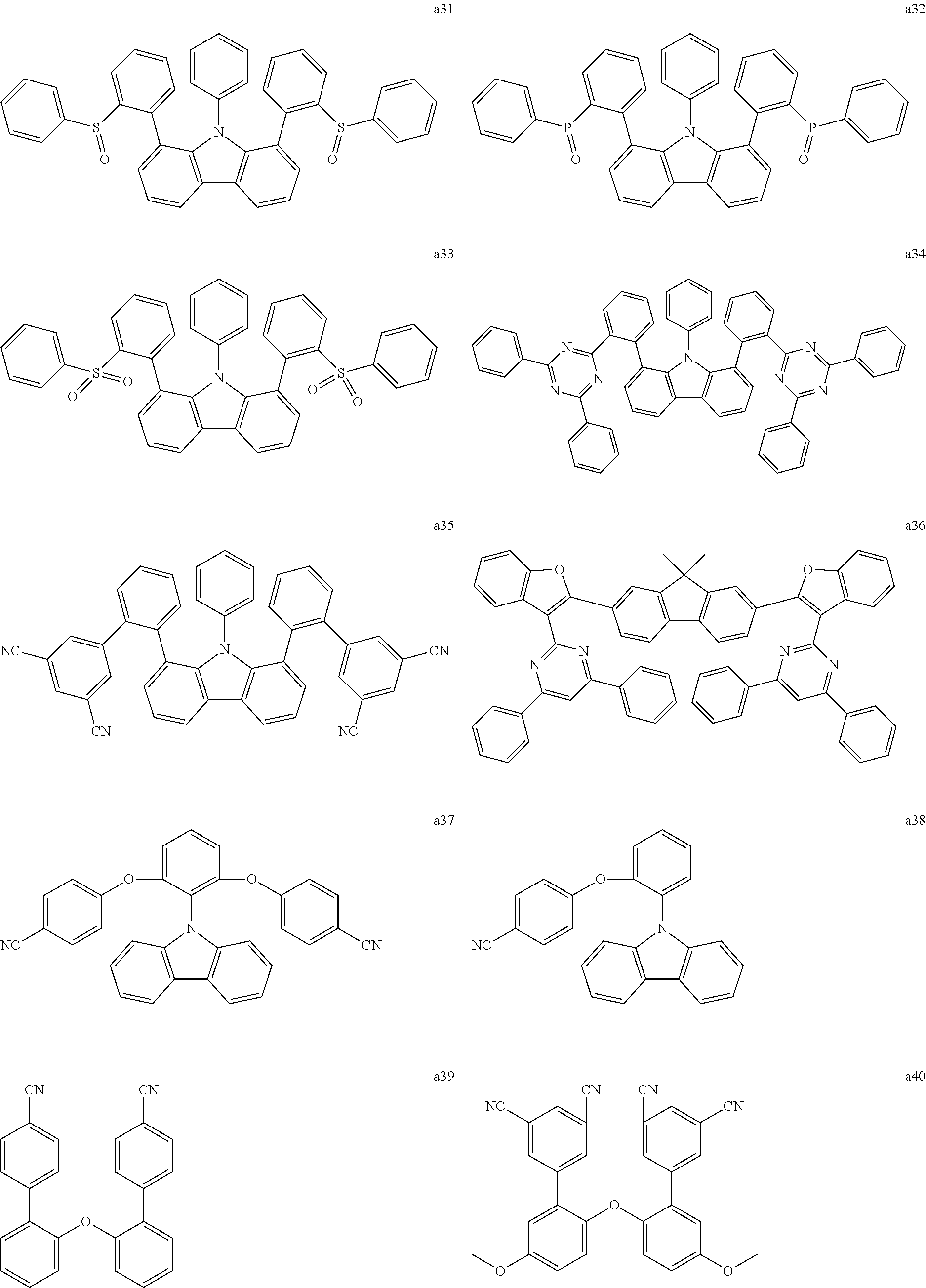

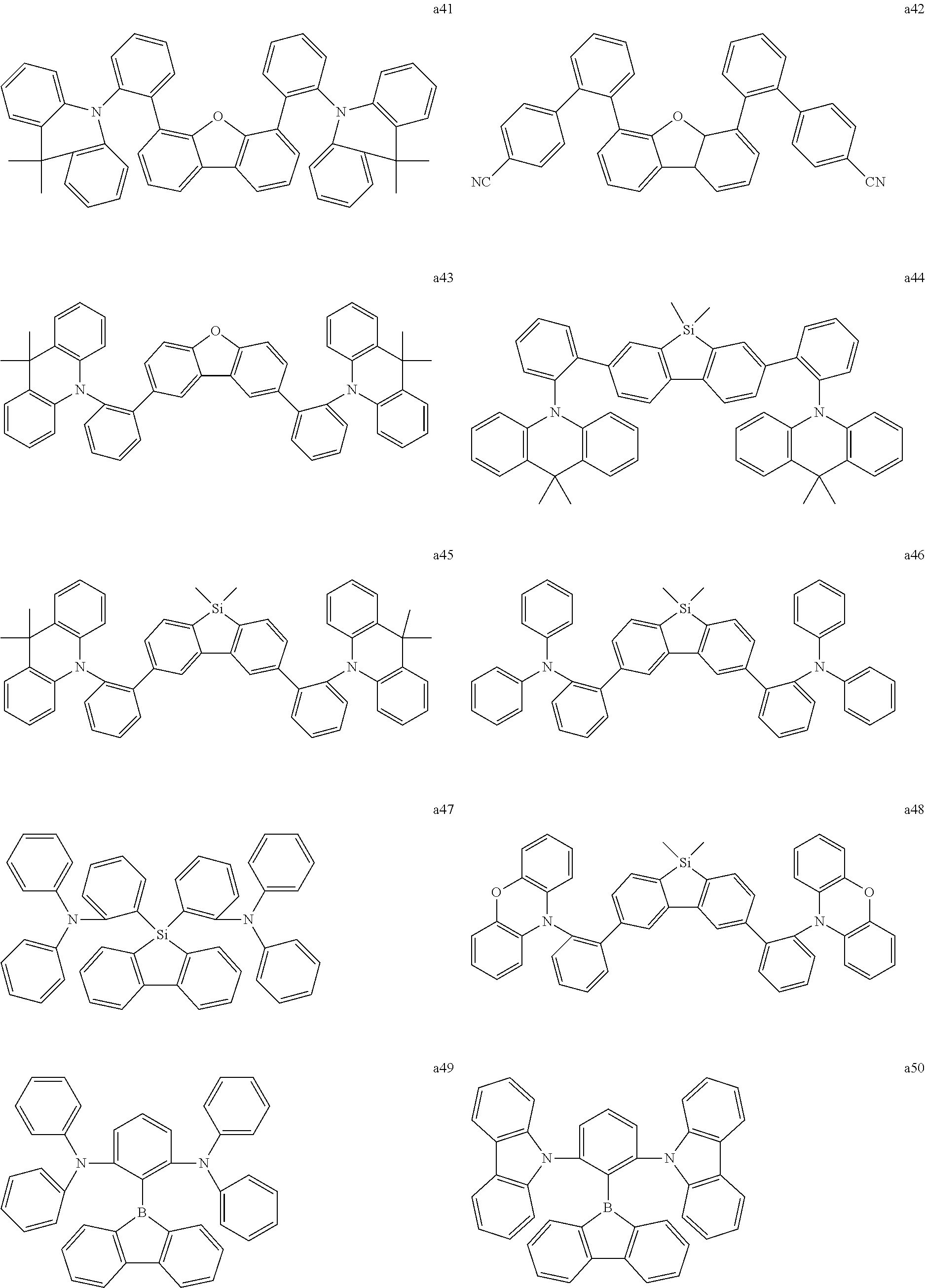

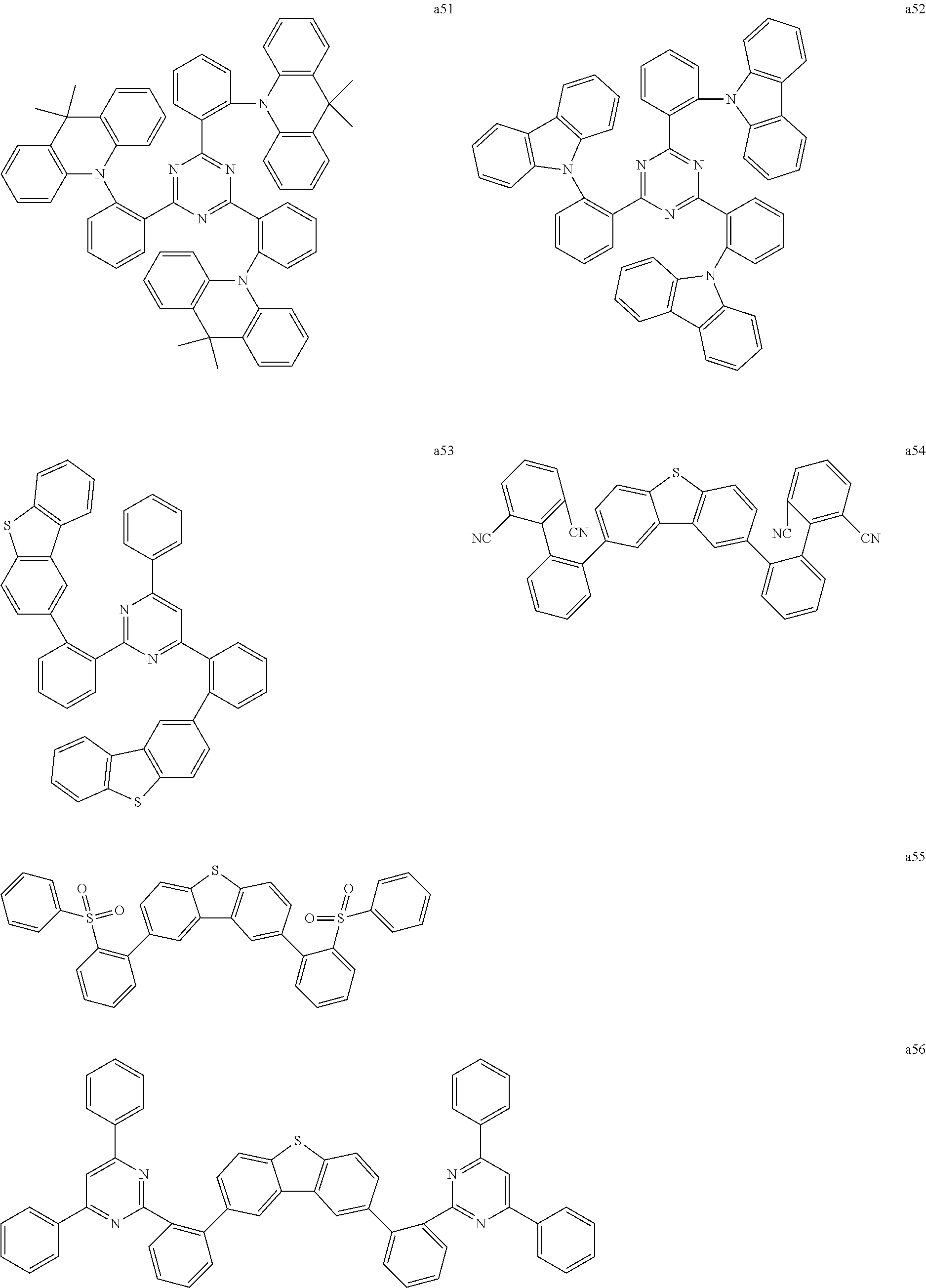

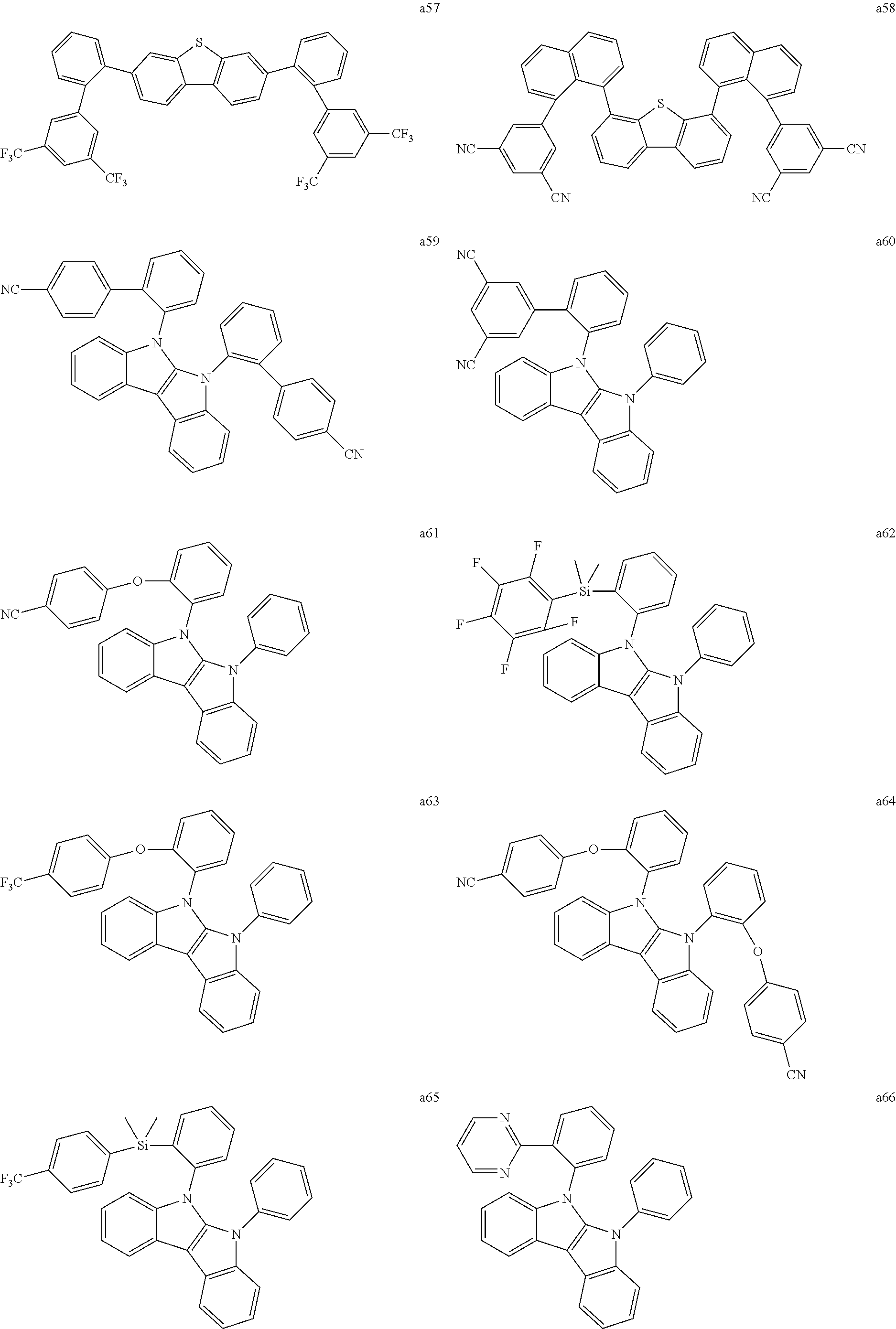

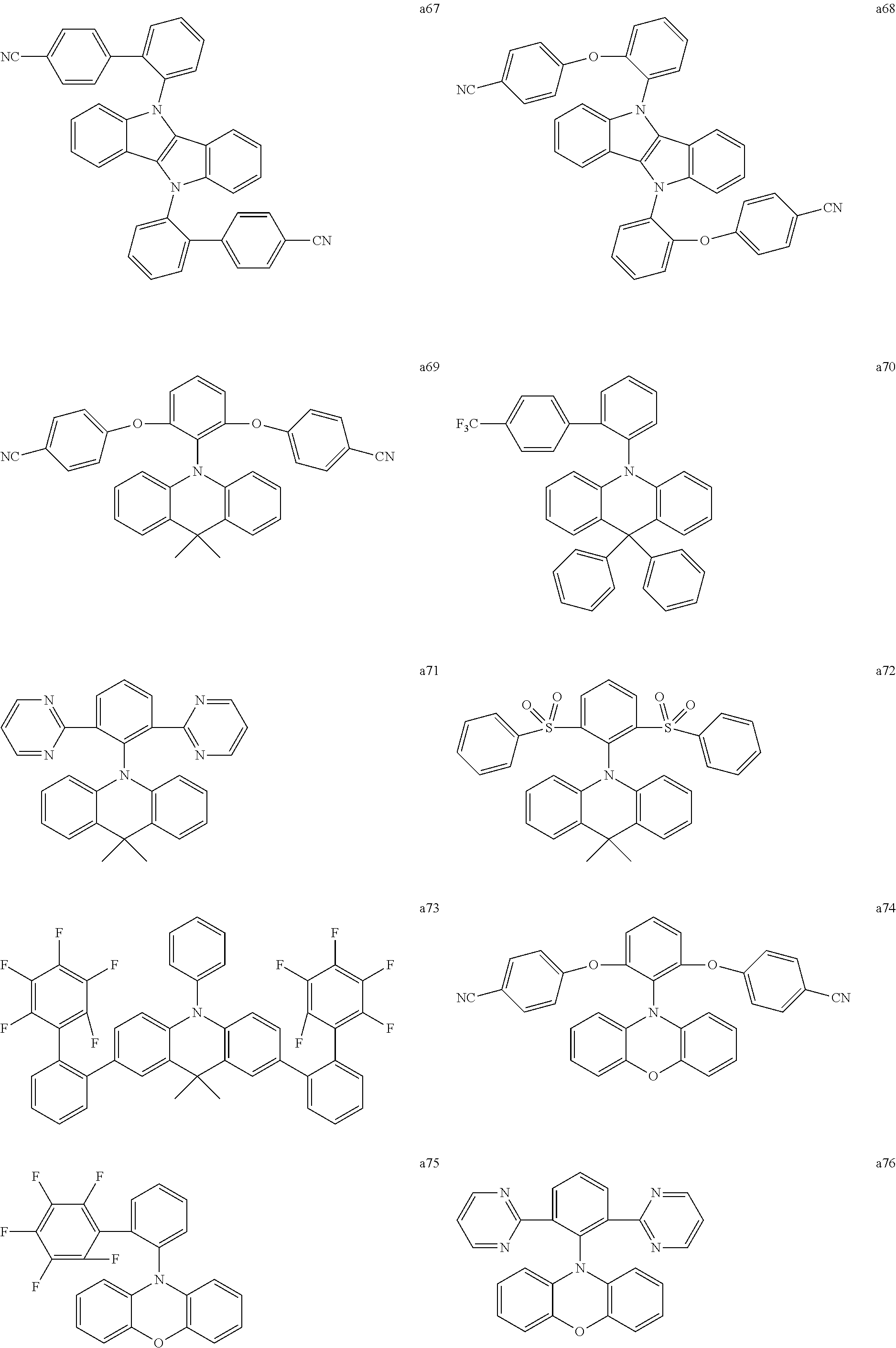

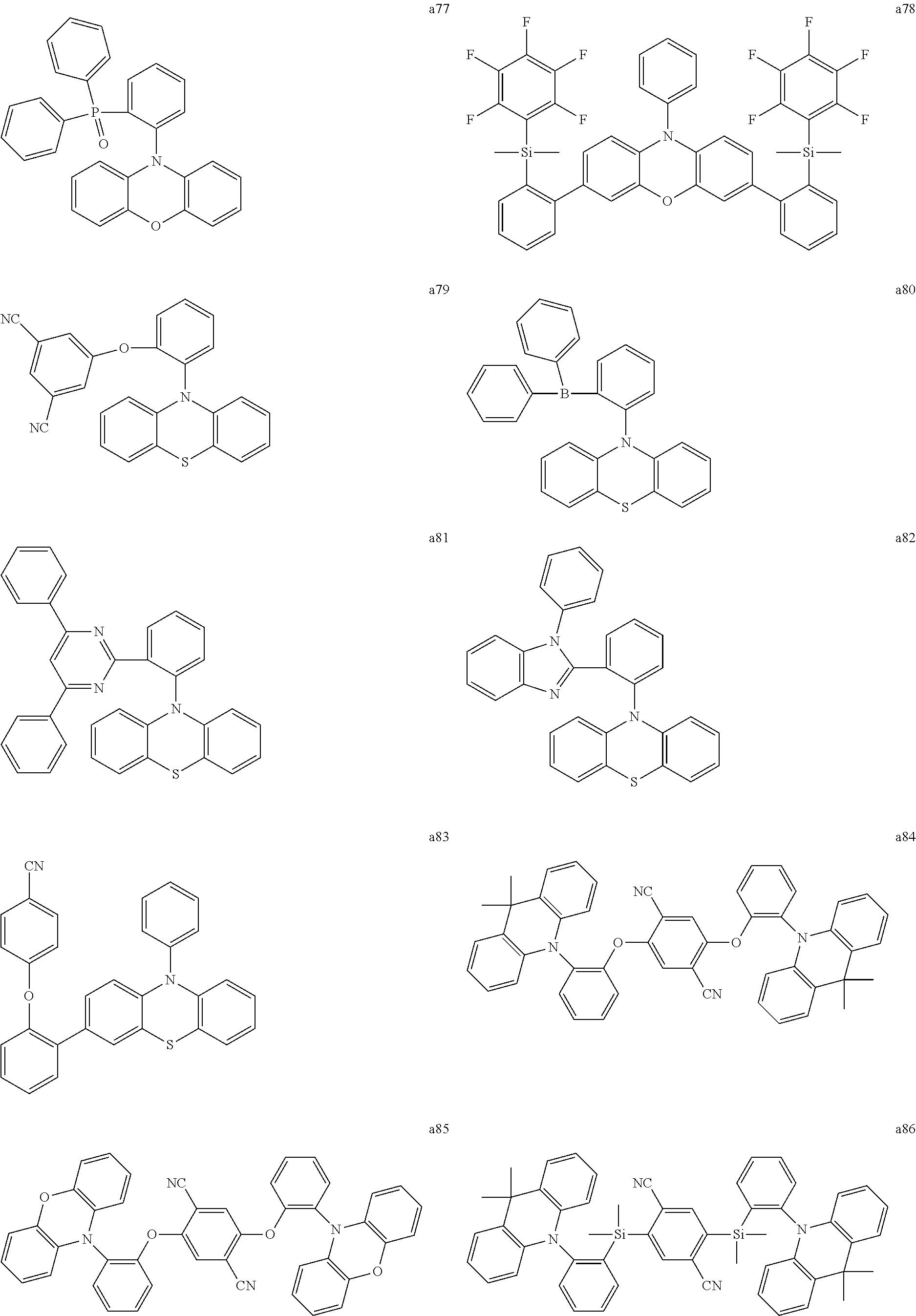

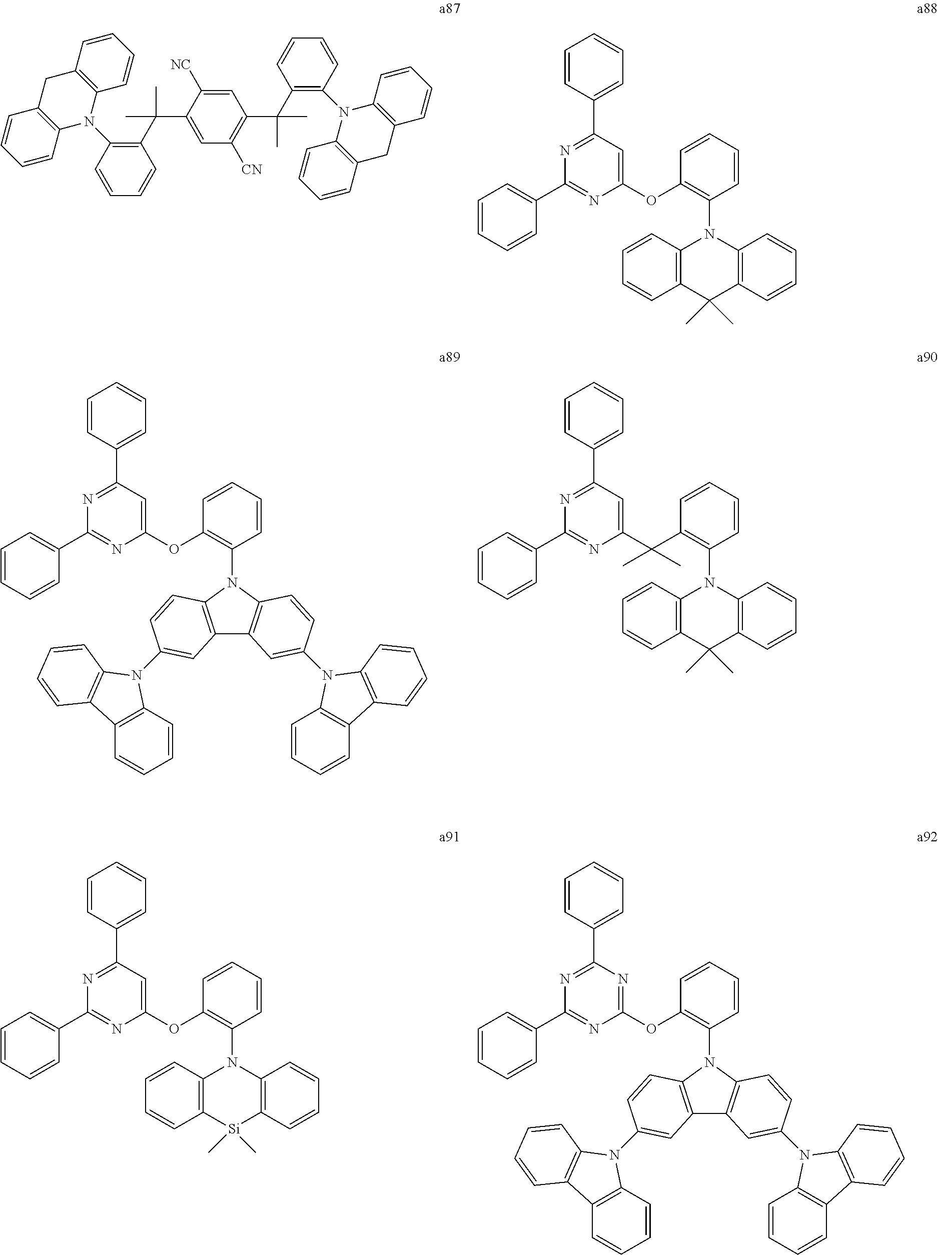

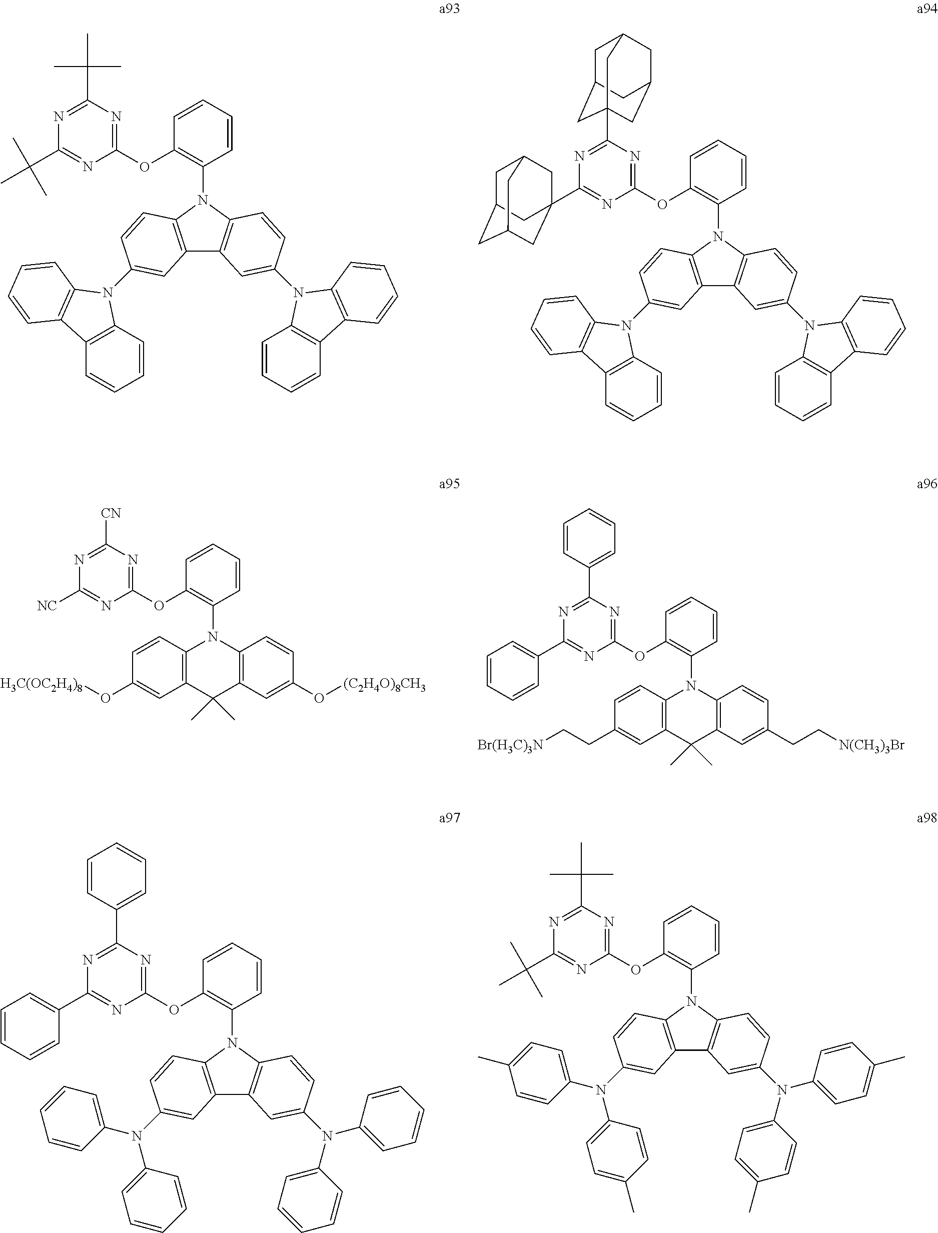

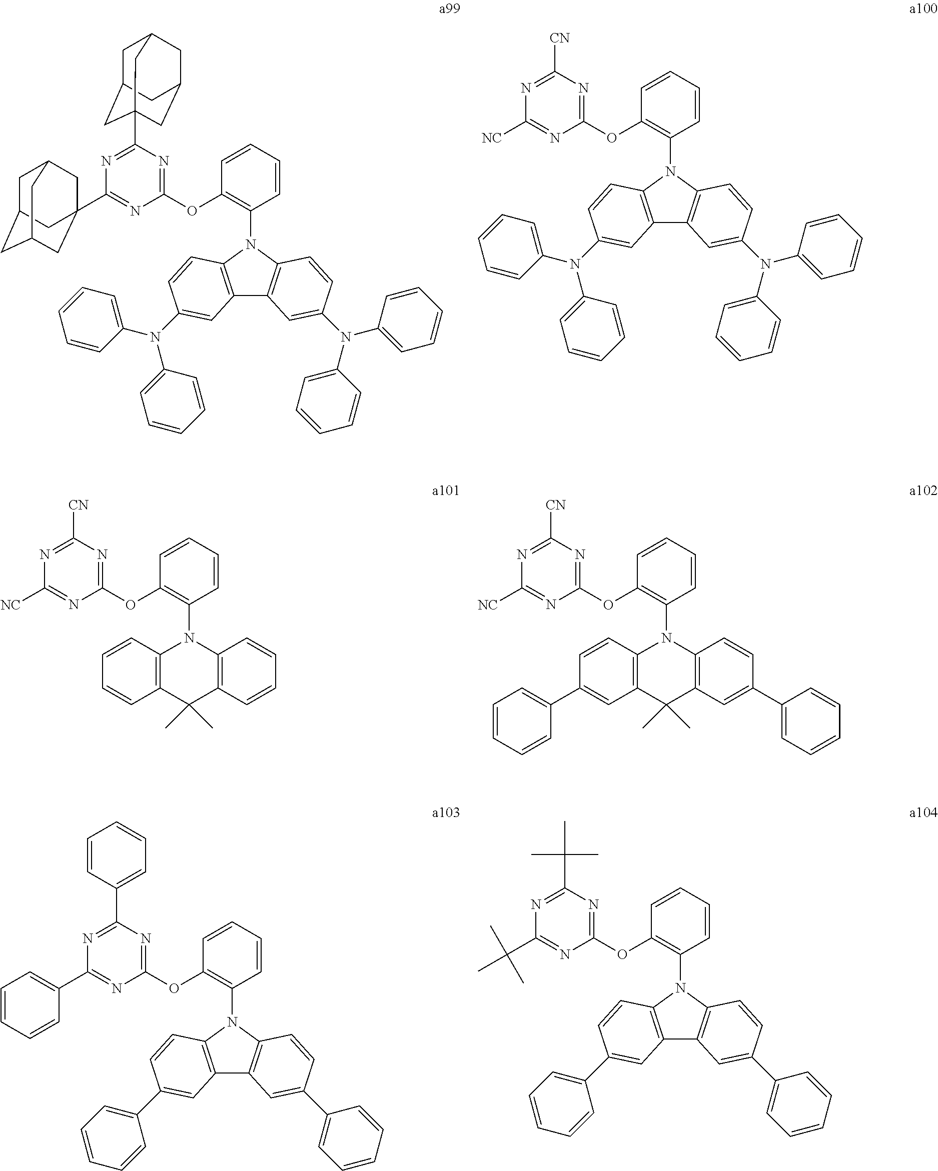

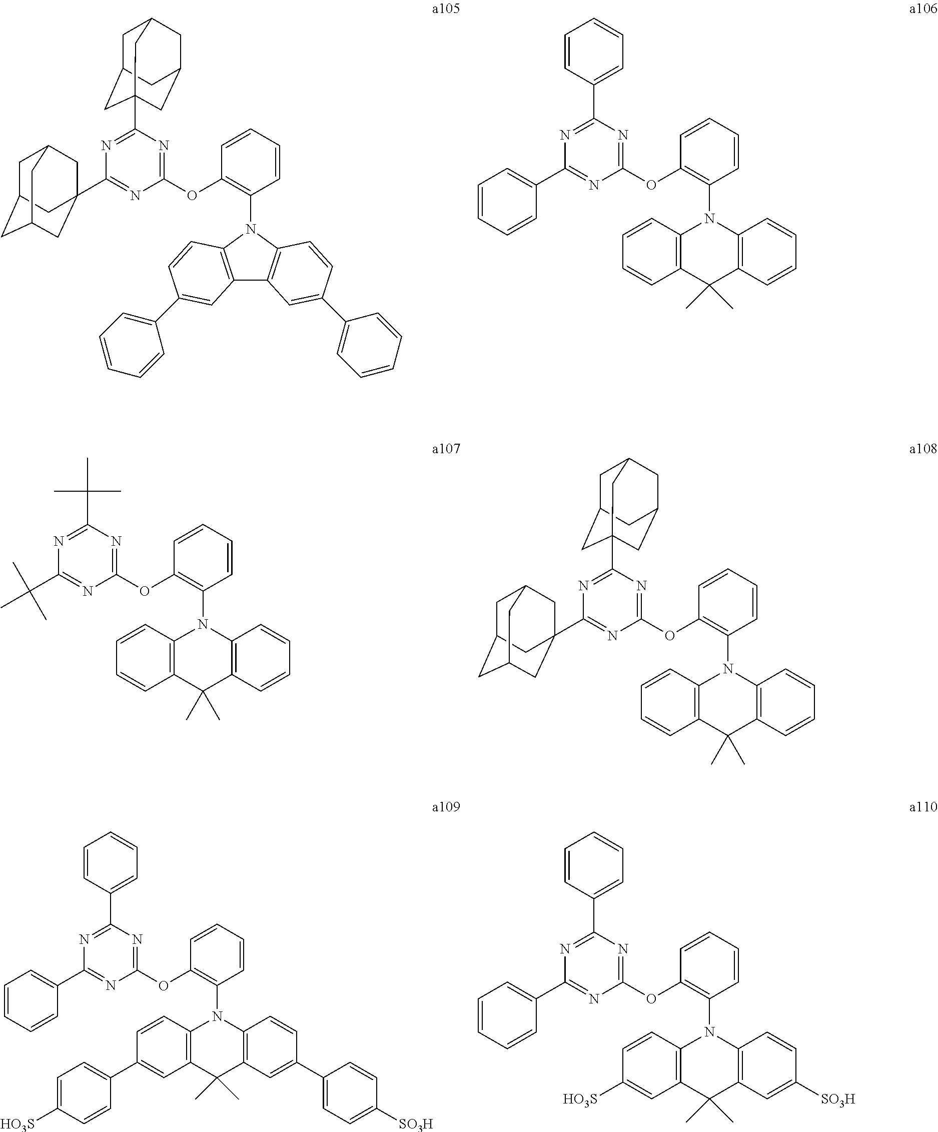

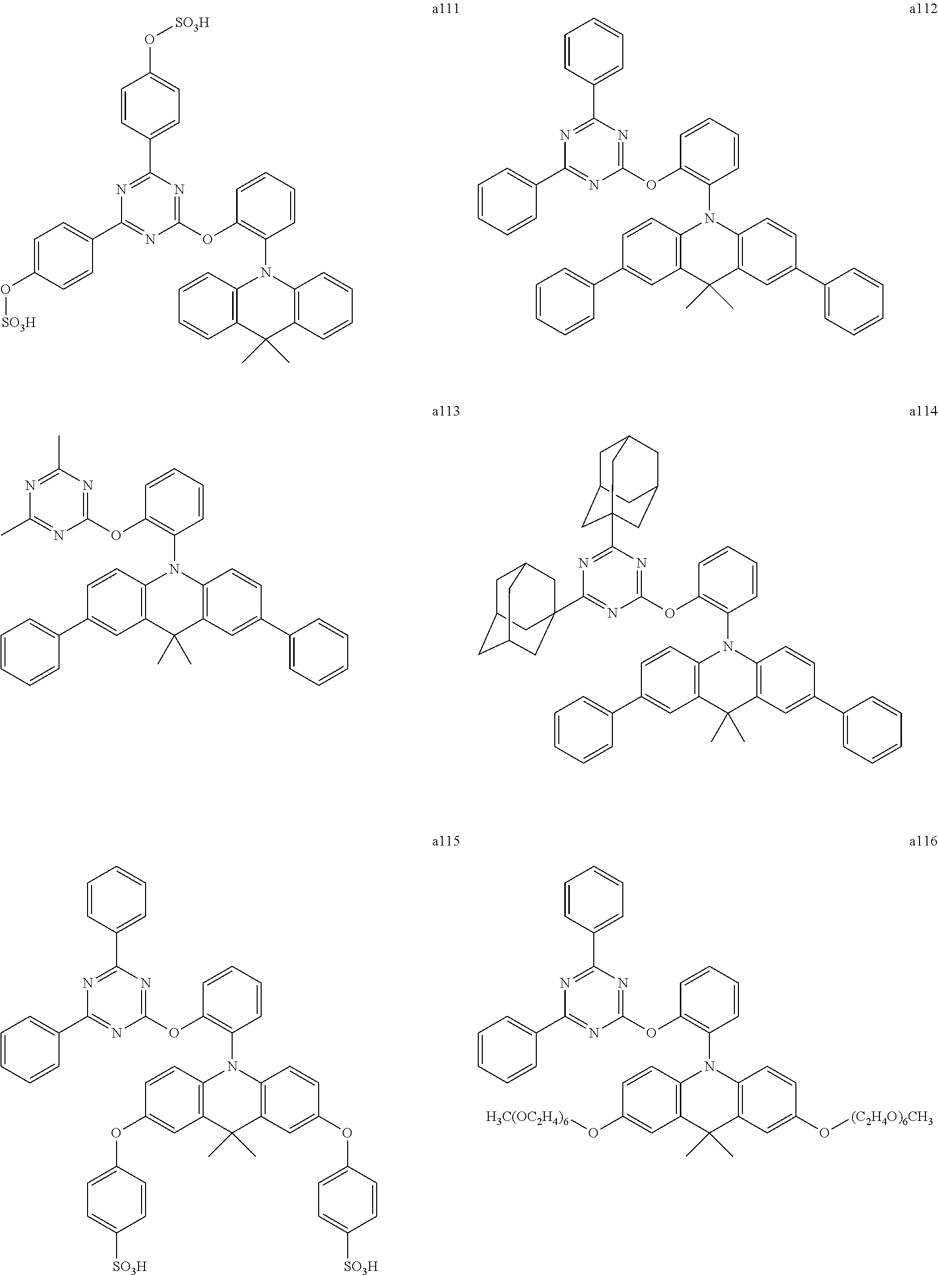

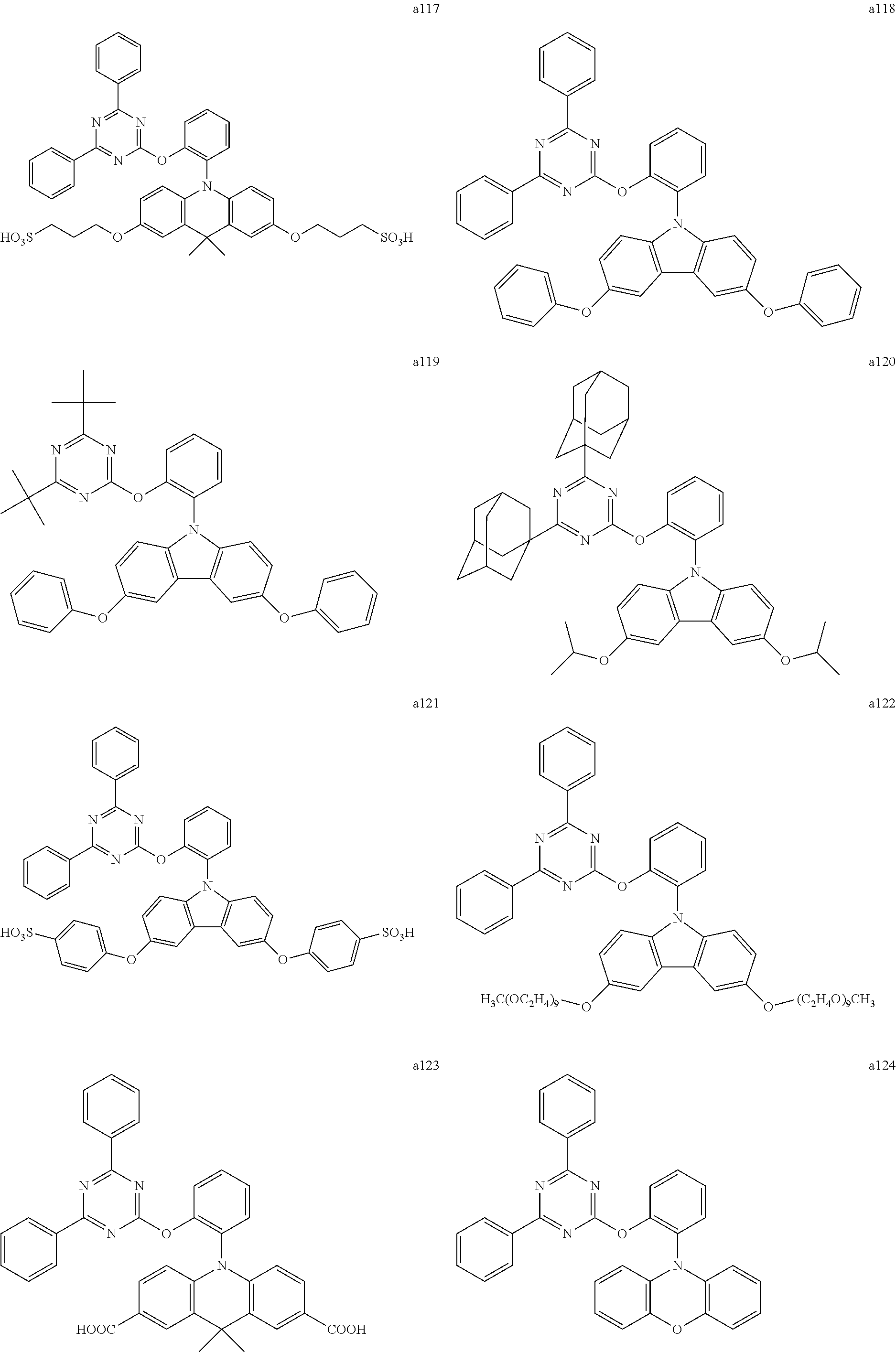

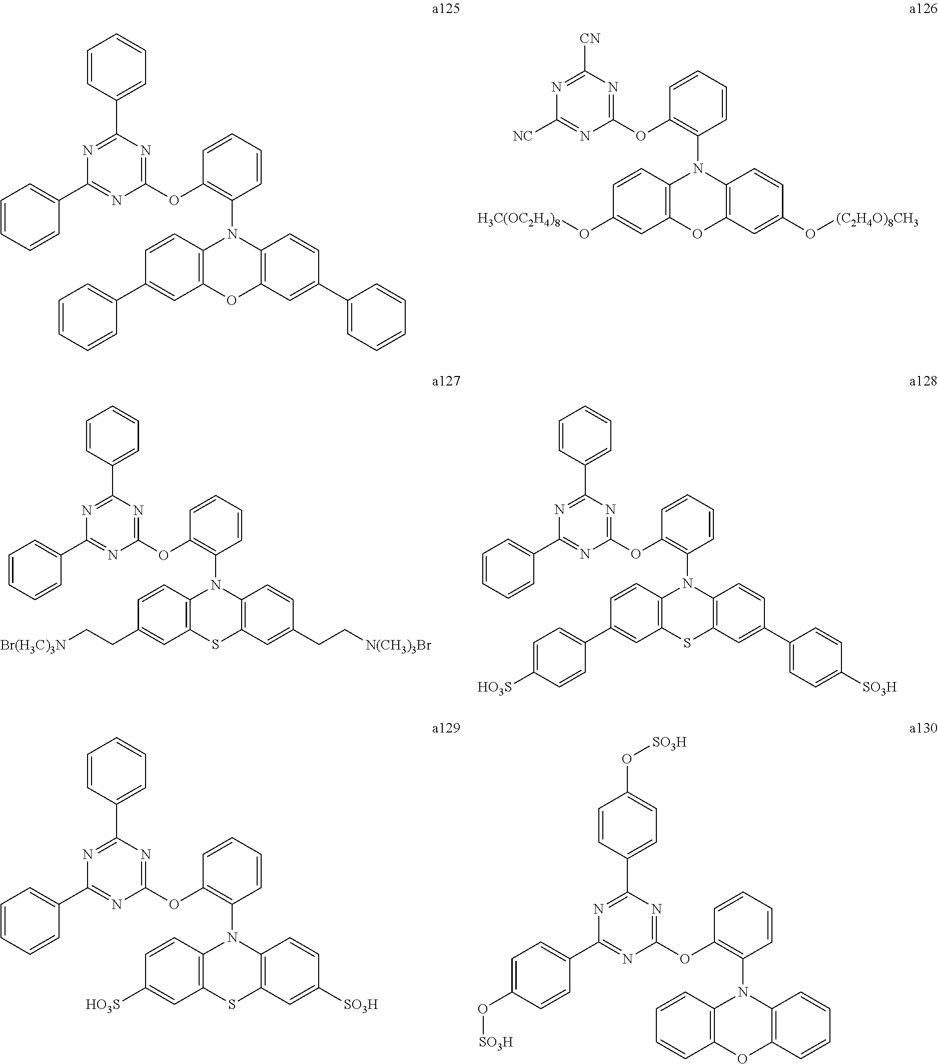

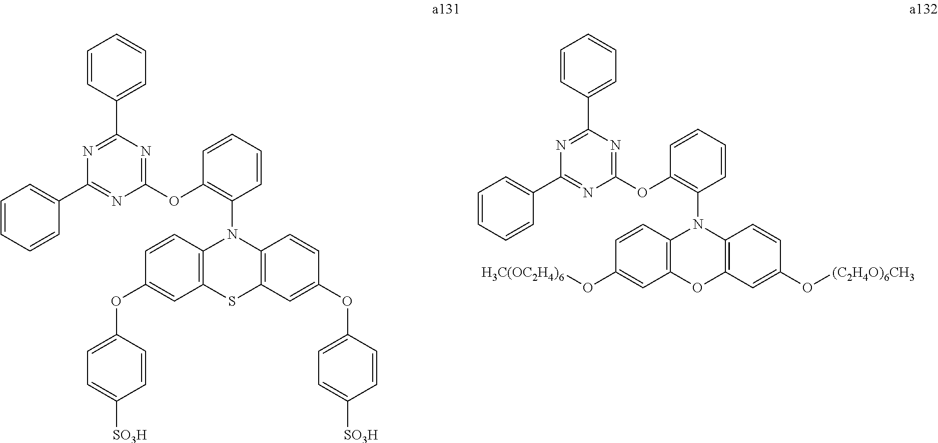

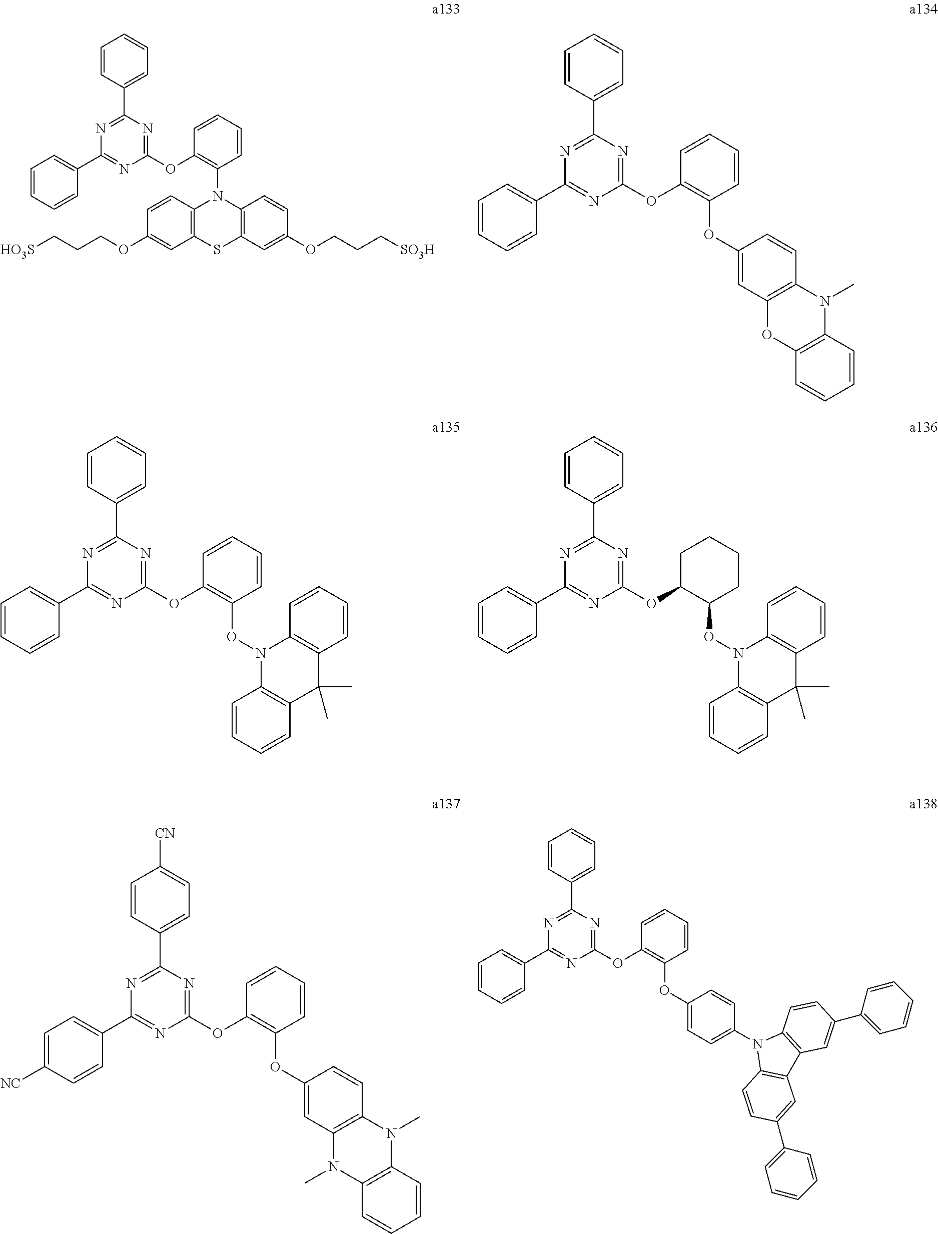 [SMILES: O=S(=O)(O)CCCOc1ccc2c(c1)Sc1cc(OCCCS(=O)(=O)O)ccc1N2c1ccccc1Oc1nc(-c2ccccc2)nc(-c2ccccc2)n1 ; CN1c2ccccc2Oc2cc(Oc3ccccc3Oc3nc(-c4ccccc4)nc(-c4ccccc4)n3)ccc21 ; CC1(C)c2ccccc2N(Oc2ccccc2Oc2nc(-c3ccccc3)nc(-c3ccccc3)n2)c2ccccc21 ; CC1(C)c2ccccc2N(O[C@@H]2CCCC[C@@H]2Oc2nc(-c3ccccc3)nc(-c3ccccc3)n2)c2ccccc21 ; CN1c2ccccc2N(C)c2cc(Oc3ccccc3Oc3nc(-c4ccc(C#N)cc4)nc(-c4ccc(C#N)cc4)n3)ccc21 ; c1ccc(-c2ccc3c(c2)c2cc(-c4ccccc4)ccc2n3-c2ccc(Oc3ccccc3Oc3nc(-c4ccccc4)nc(-c4ccccc4)n3)cc2)cc1]

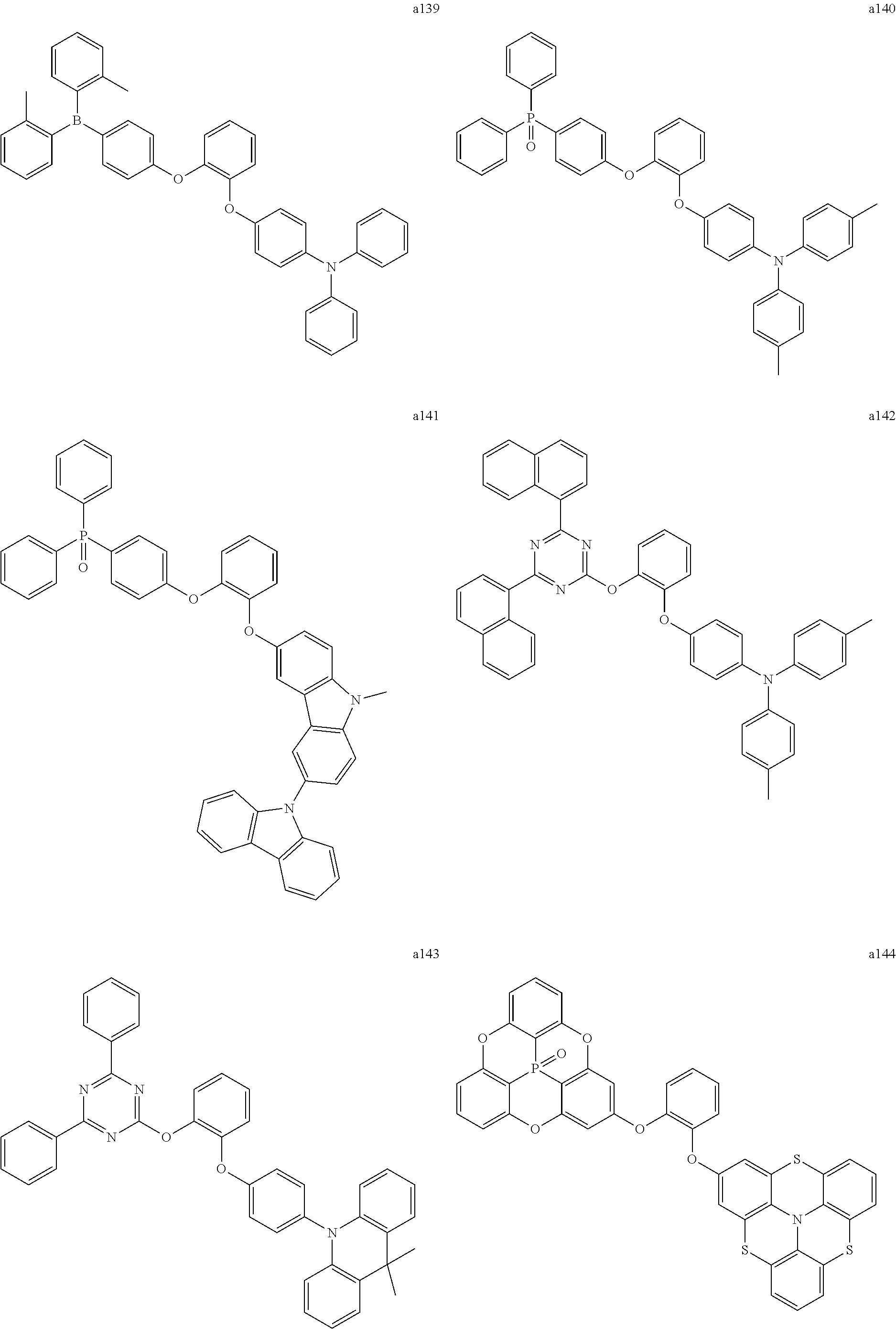

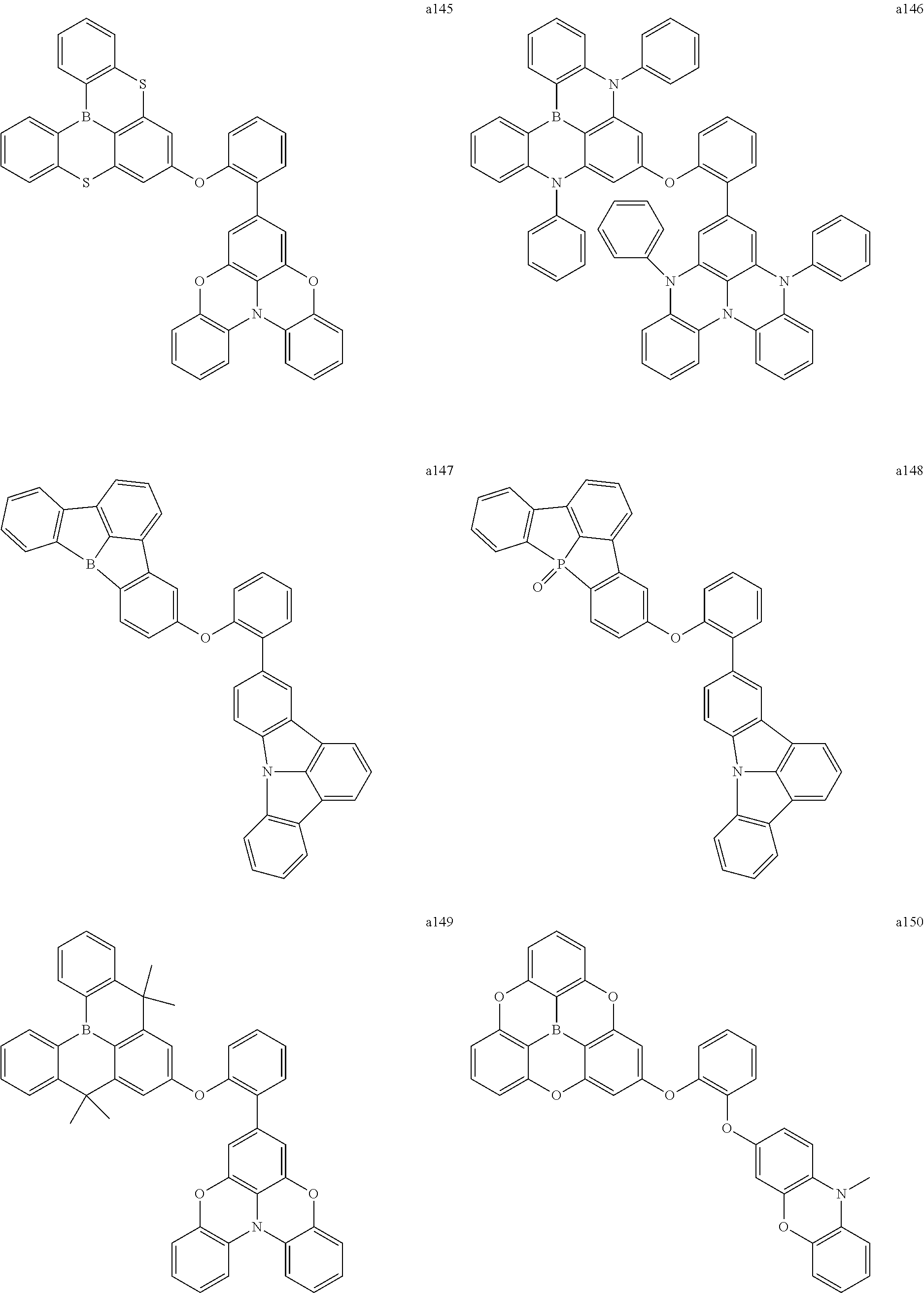

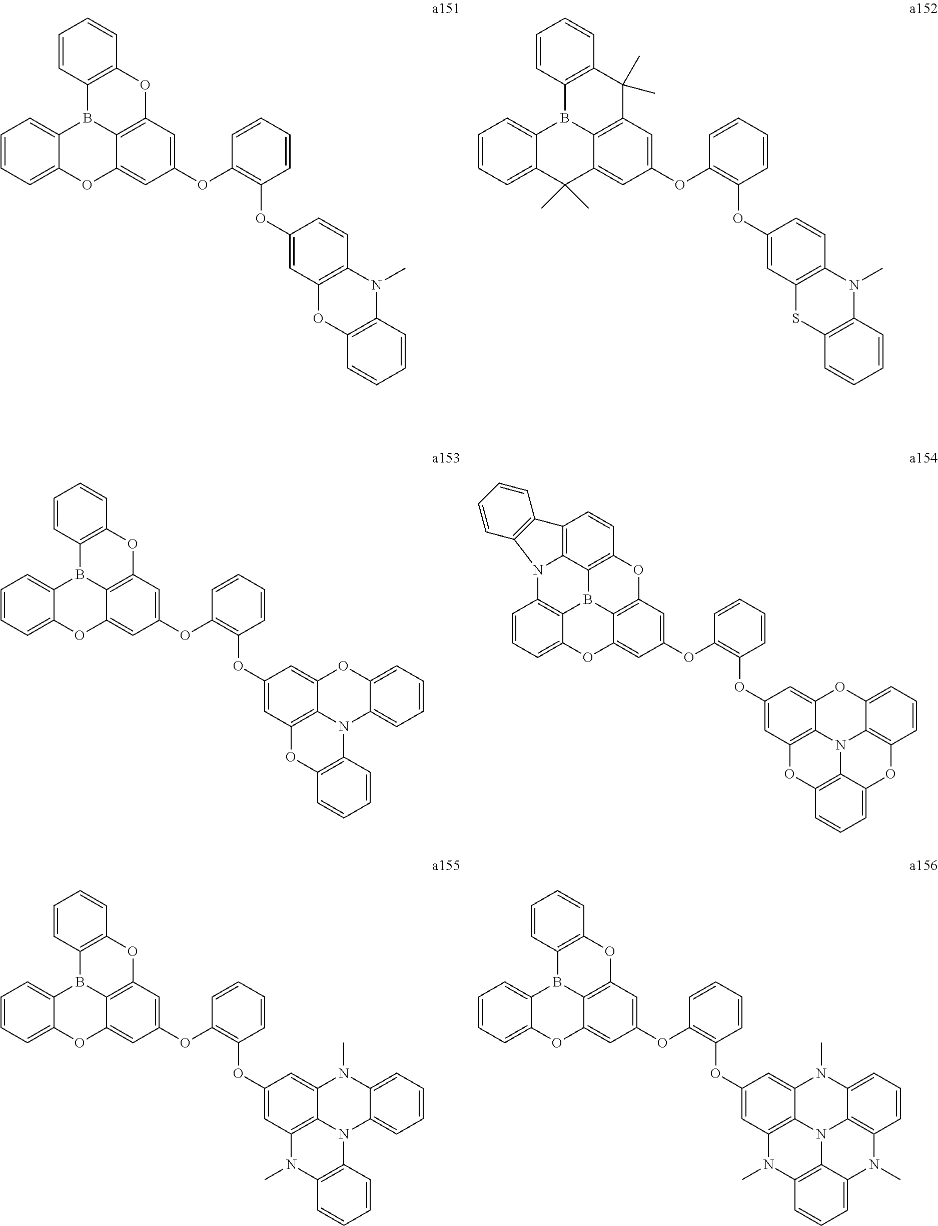

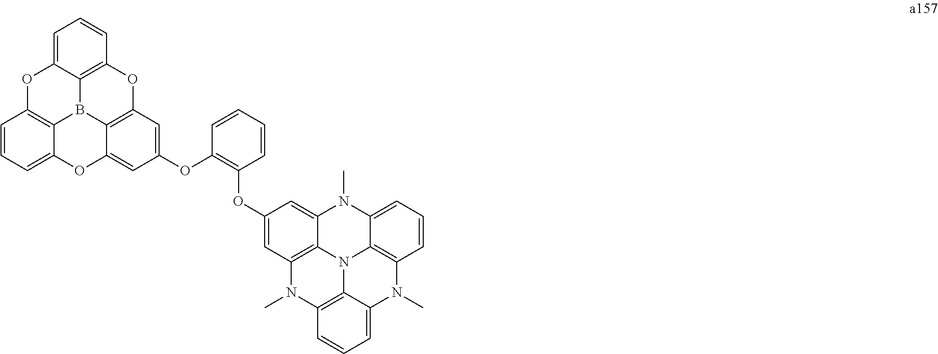 [SMILES: CN1c2cccc3c2N2c4c1cccc4N(C)c1cc(Oc4ccccc4Oc4cc5c6c(c4)Oc4cccc7c4B6c4c(cccc4O5)O7)cc(c12)N3C]

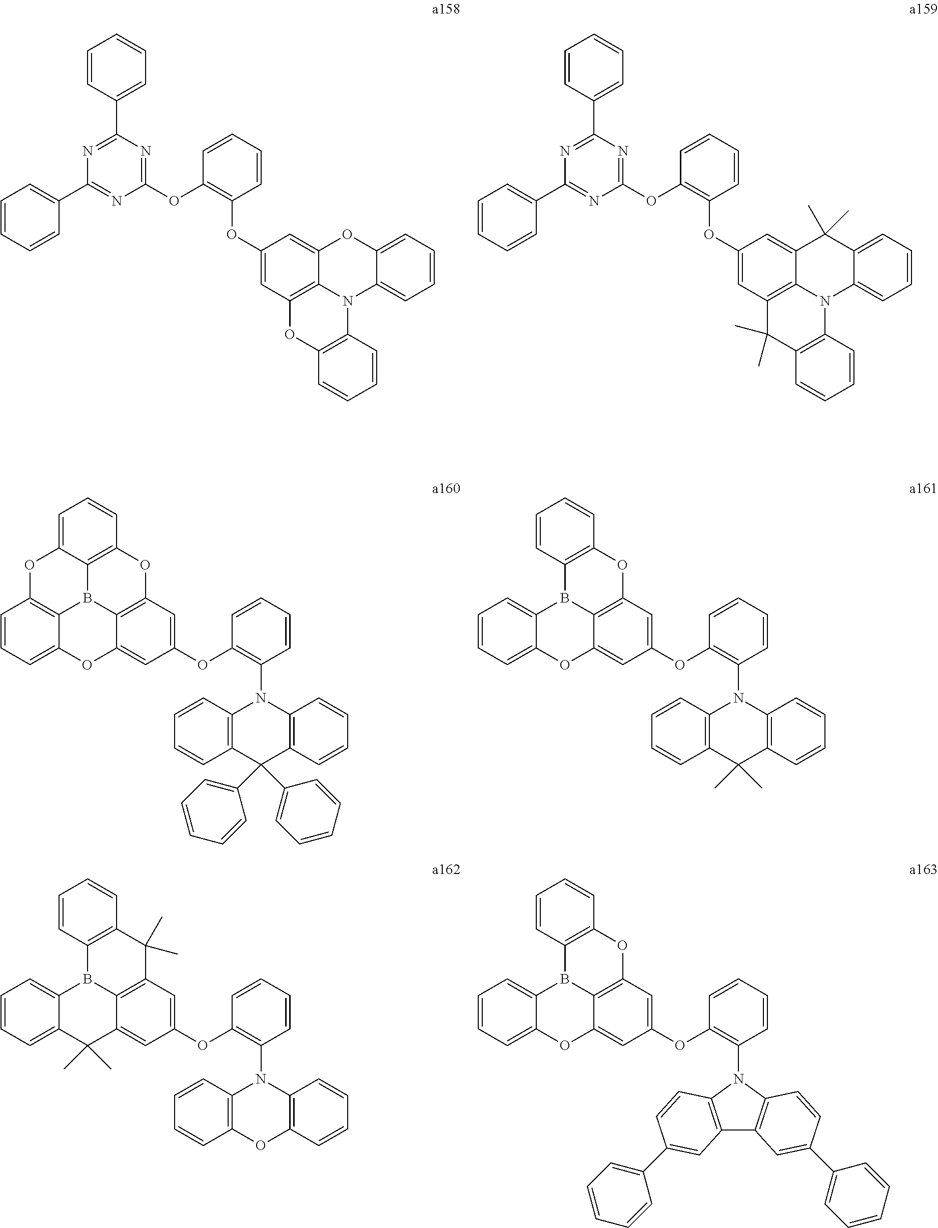 [SMILES: c1ccc(-c2nc(Oc3ccccc3Oc3cc4c5c(c3)Oc3ccccc3N5c3ccccc3O4)nc(-c3ccccc3)n2)cc1 ; CC1(C)c2ccccc2N2c3ccccc3C(C)(C)c3cc(Oc4ccccc4Oc4nc(-c5ccccc5)nc(-c5ccccc5)n4)cc1c32 ; c1ccc(C2(c3ccccc3)c3ccccc3N(c3ccccc3Oc3cc4c5c(c3)Oc3cccc6c3B5c3c(cccc3O4)O6)c3ccccc32)cc1 ; CC1(C)c2ccccc2N(c2ccccc2Oc2cc3c4c(c2)Oc2ccccc2B4c2ccccc2O3)c2ccccc21 ; CC1(C)c2ccccc2B2c3ccccc3C(C)(C)c3cc(Oc4ccccc4N4c5ccccc5Oc5ccccc54)cc1c32 ; c1ccc(-c2ccc3c(c2)c2cc(-c4ccccc4)ccc2n3-c2ccccc2Oc2cc3c4c(c2)Oc2ccccc2B4c2ccccc2O3)cc1]

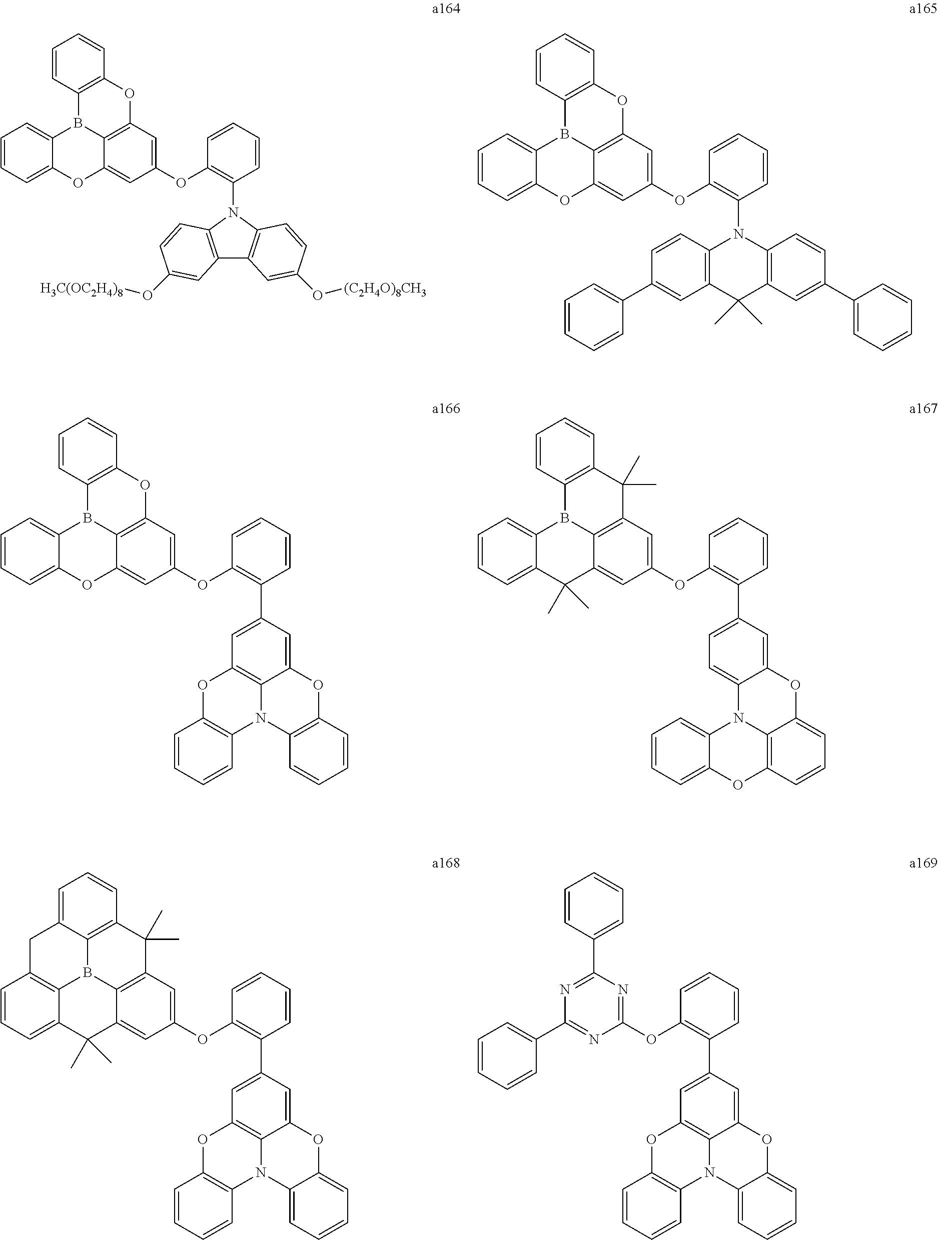

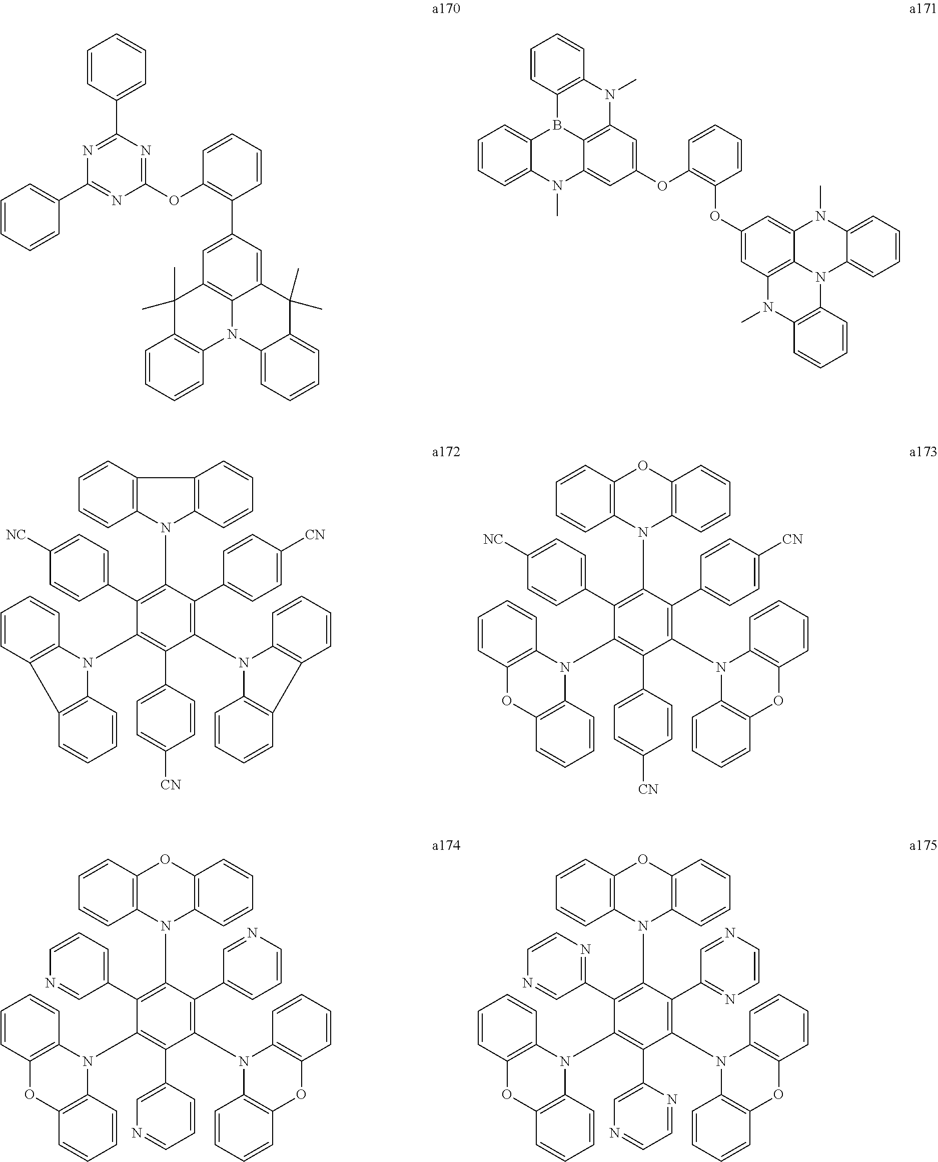

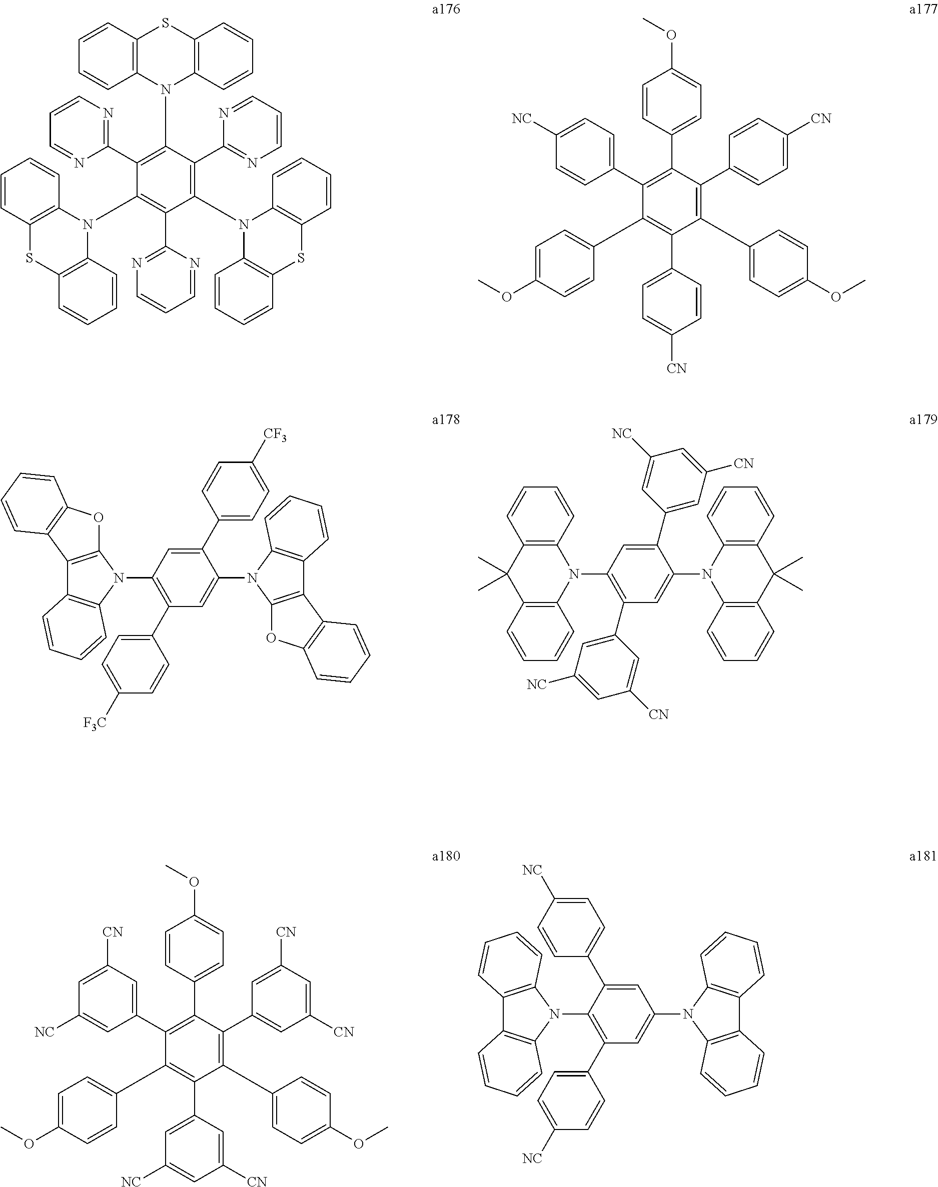

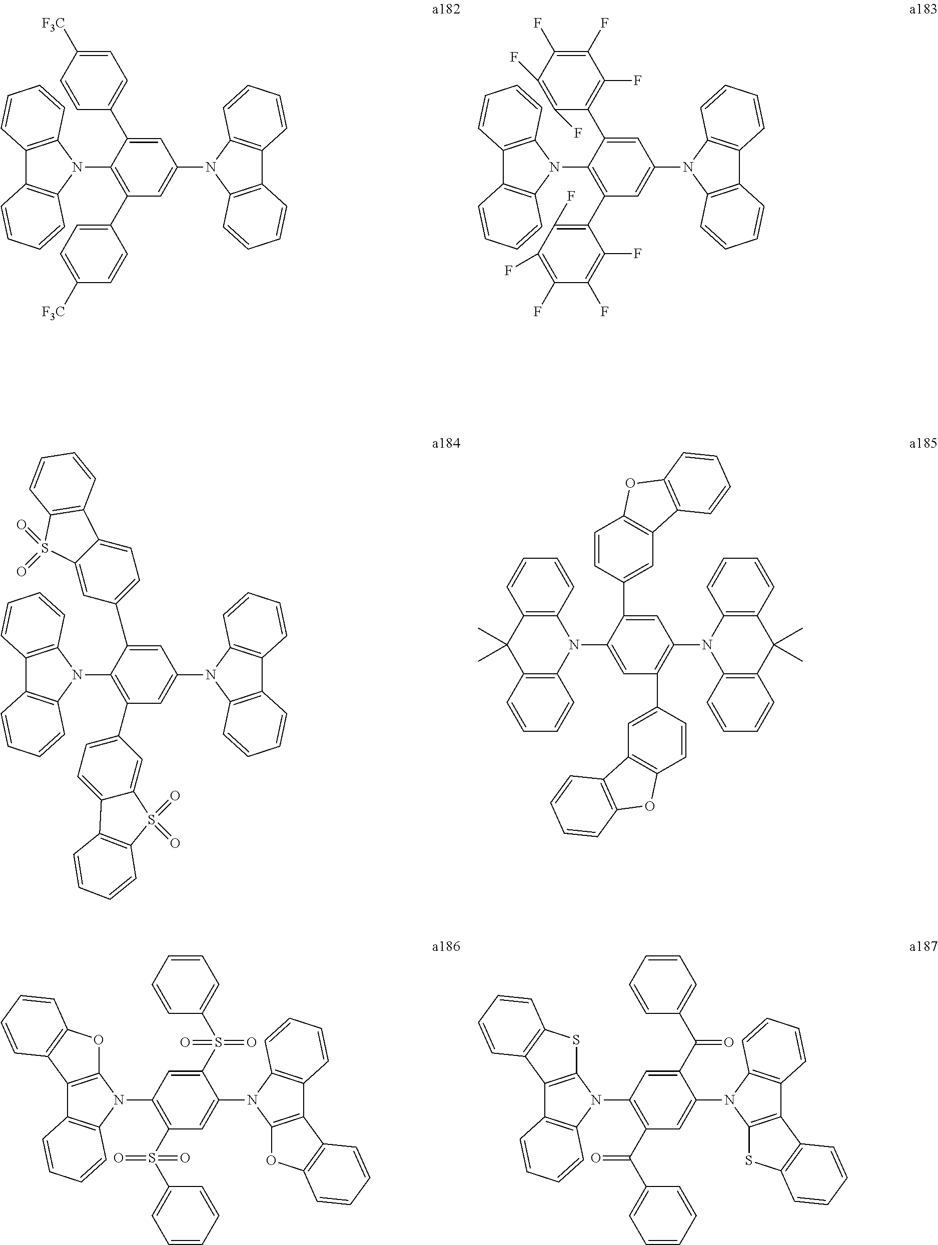

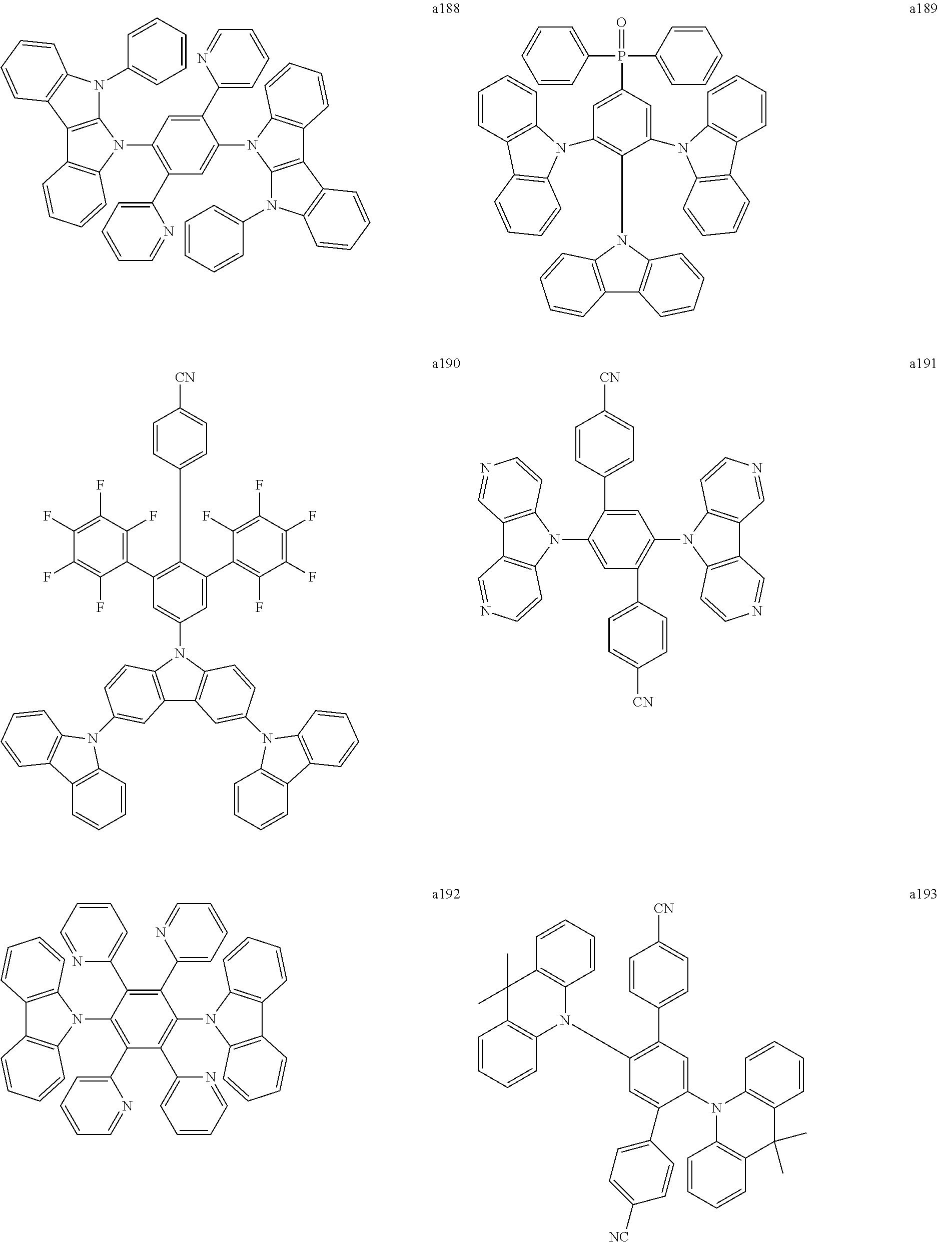

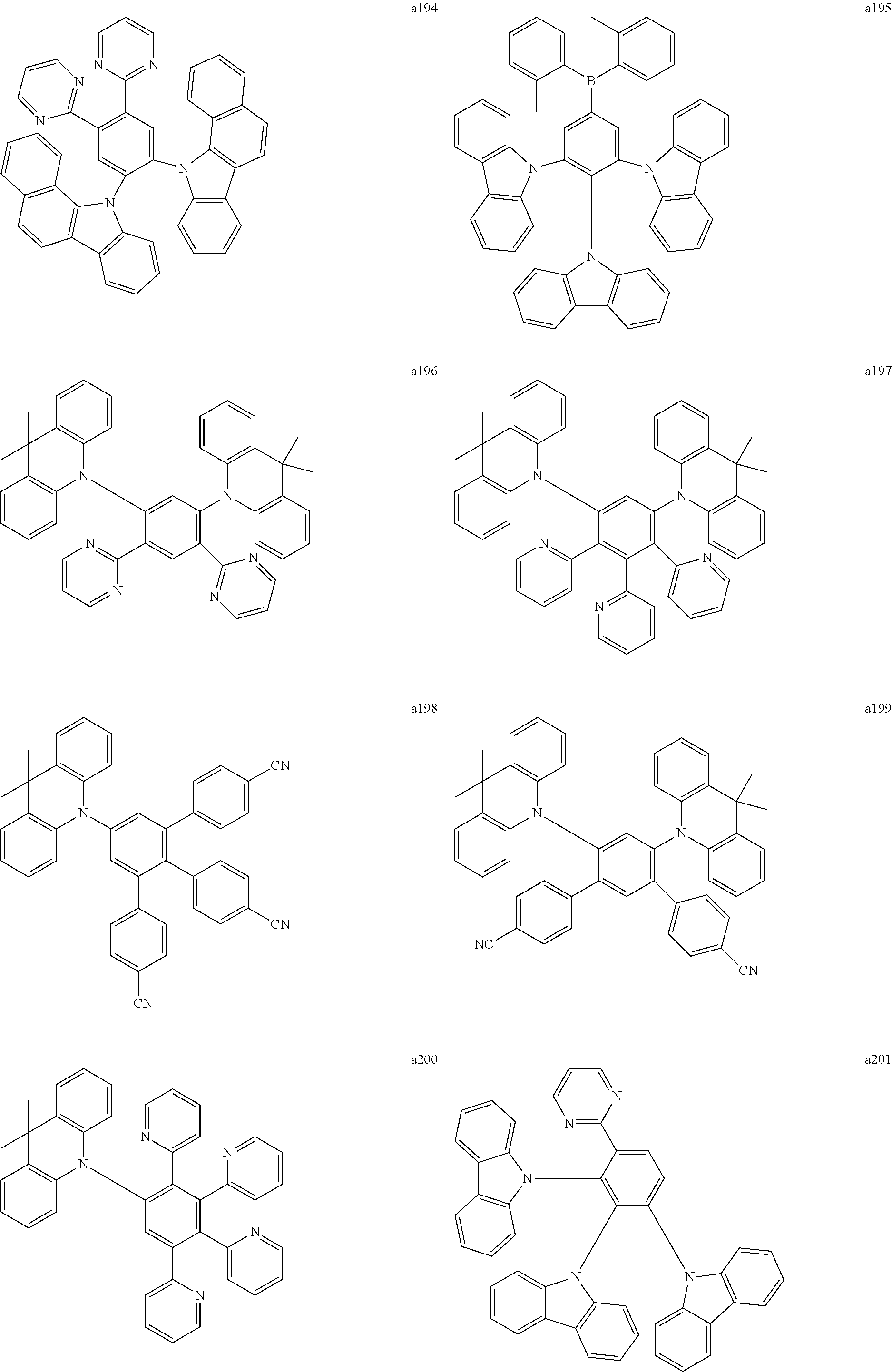

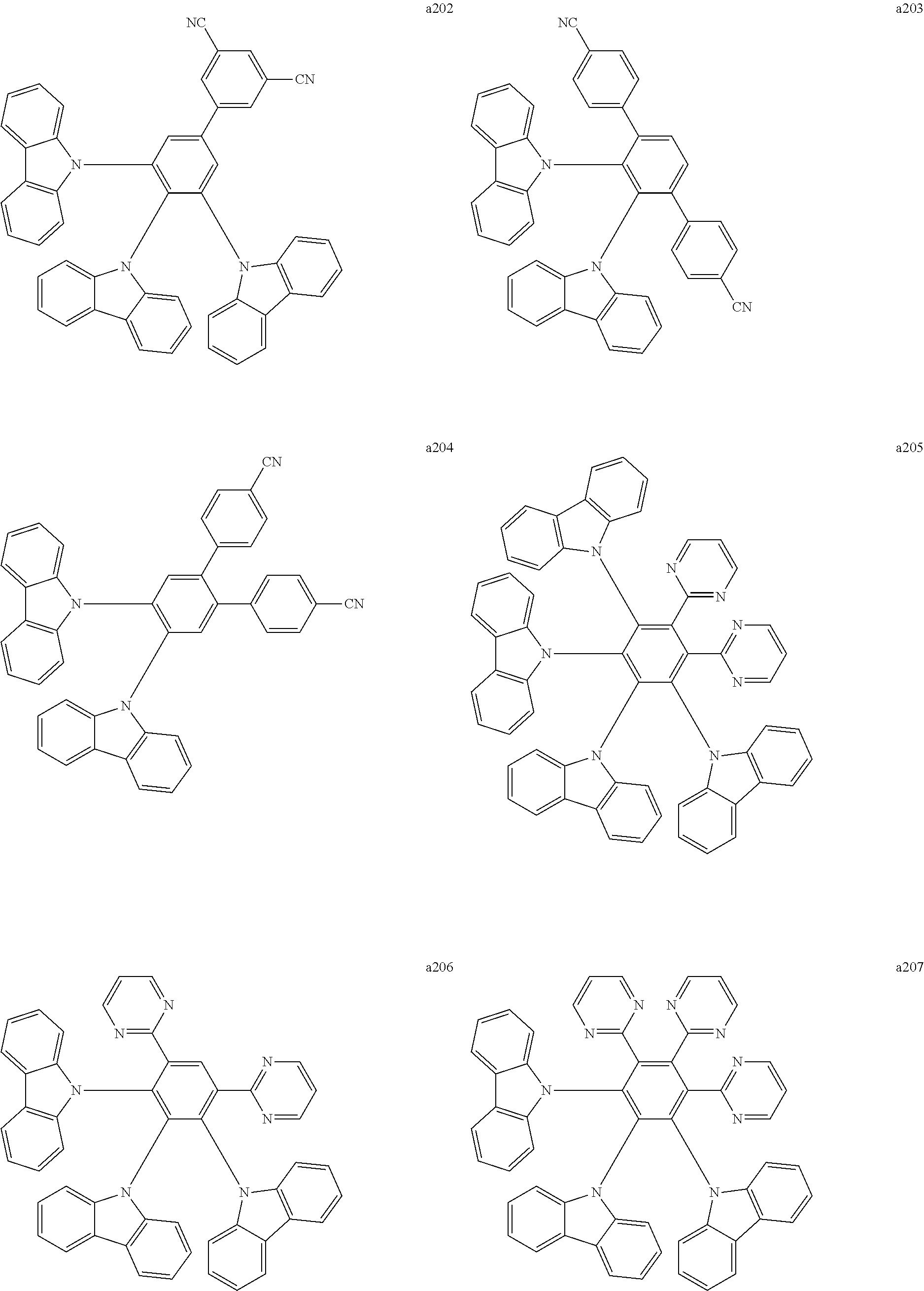

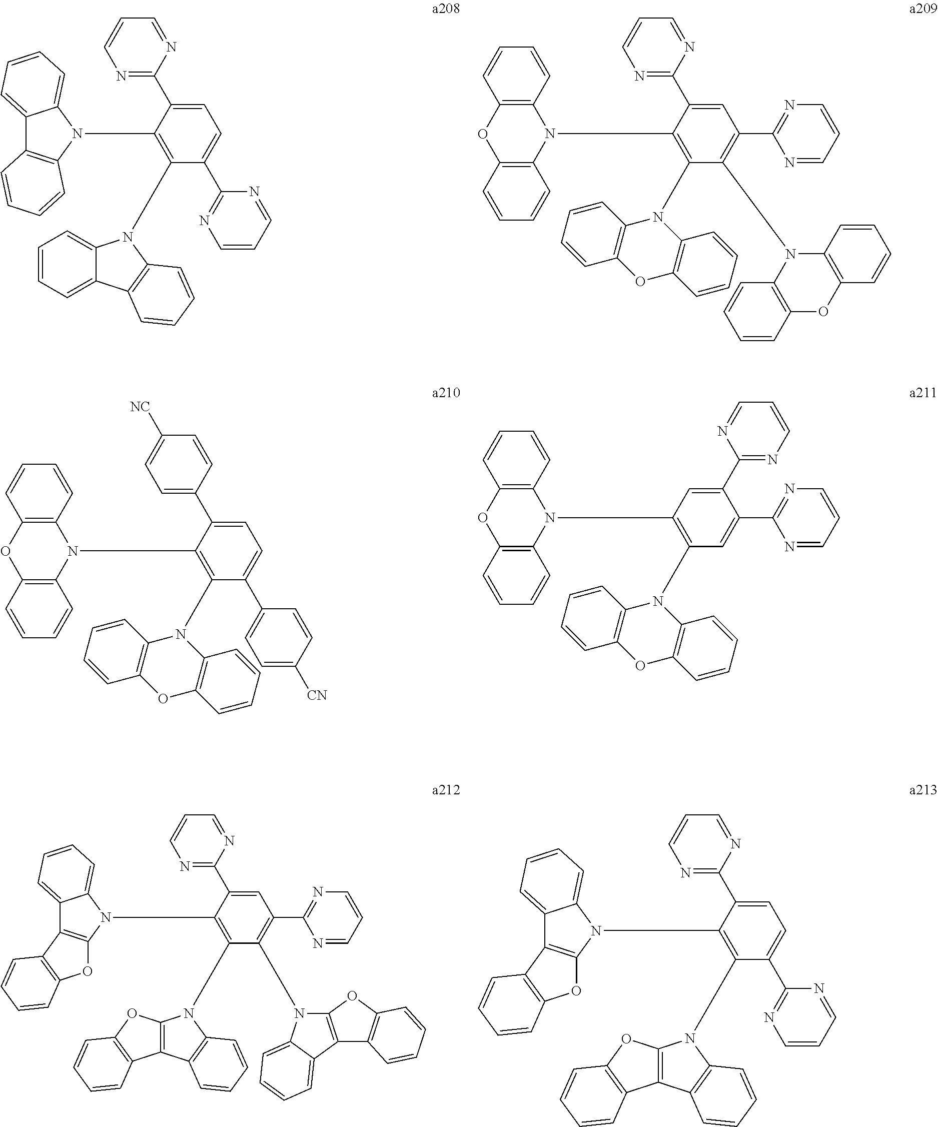

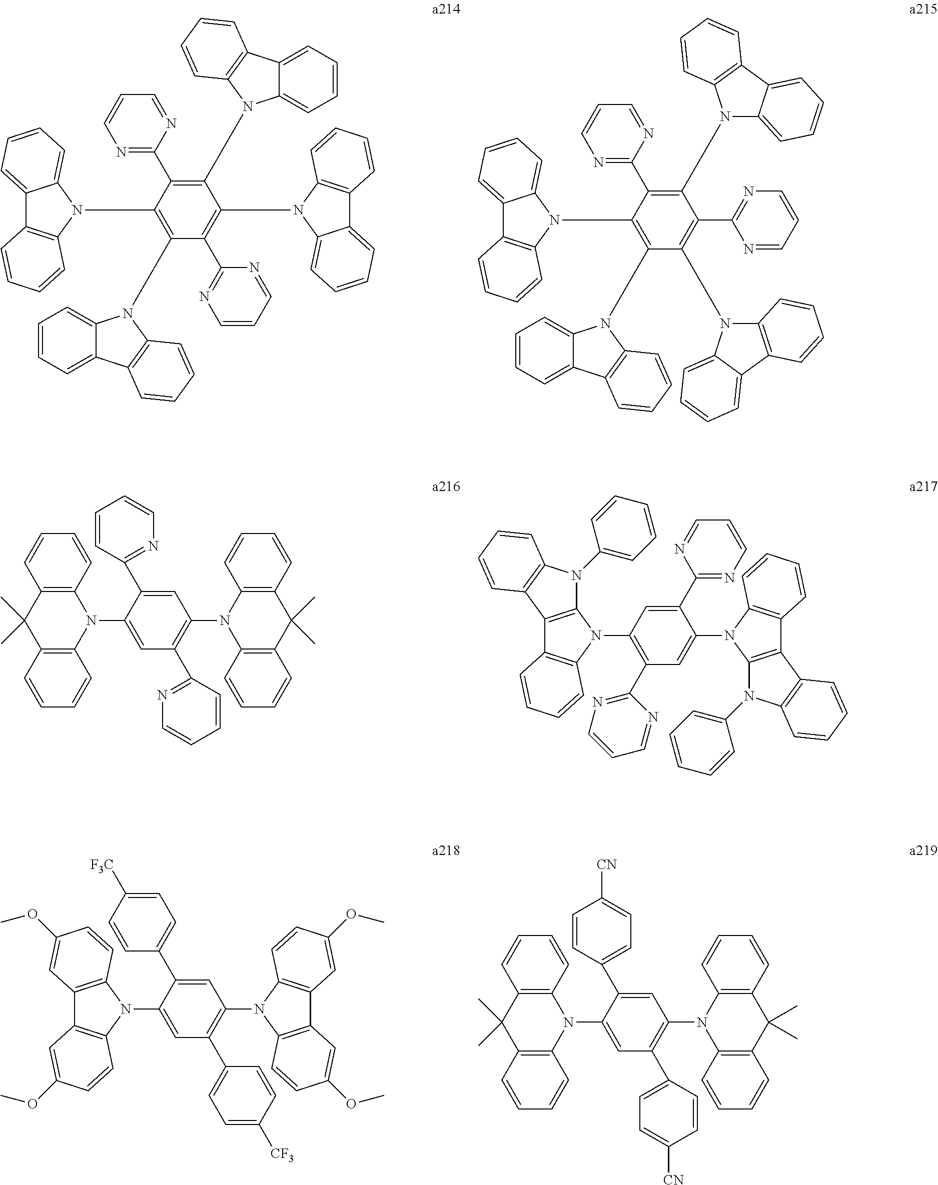

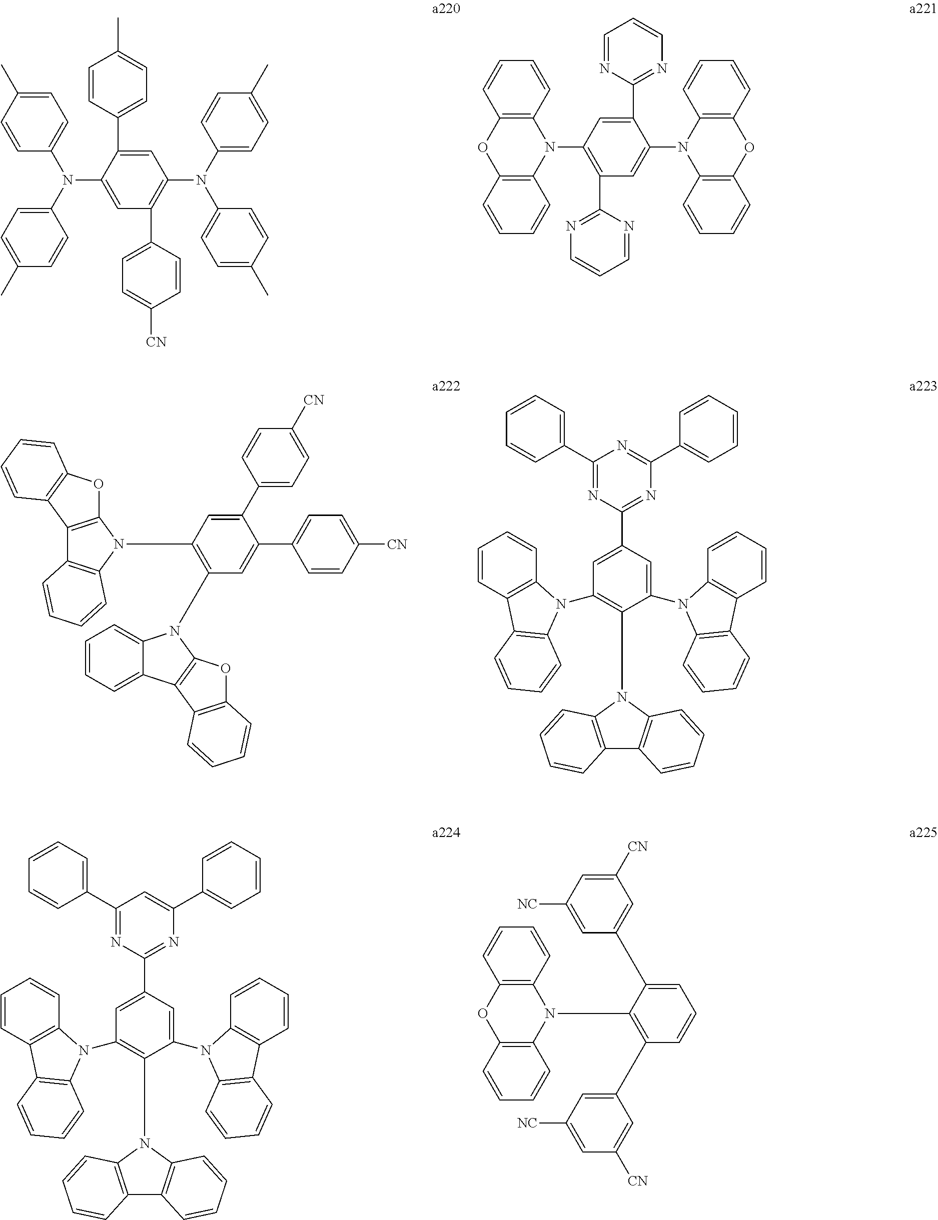

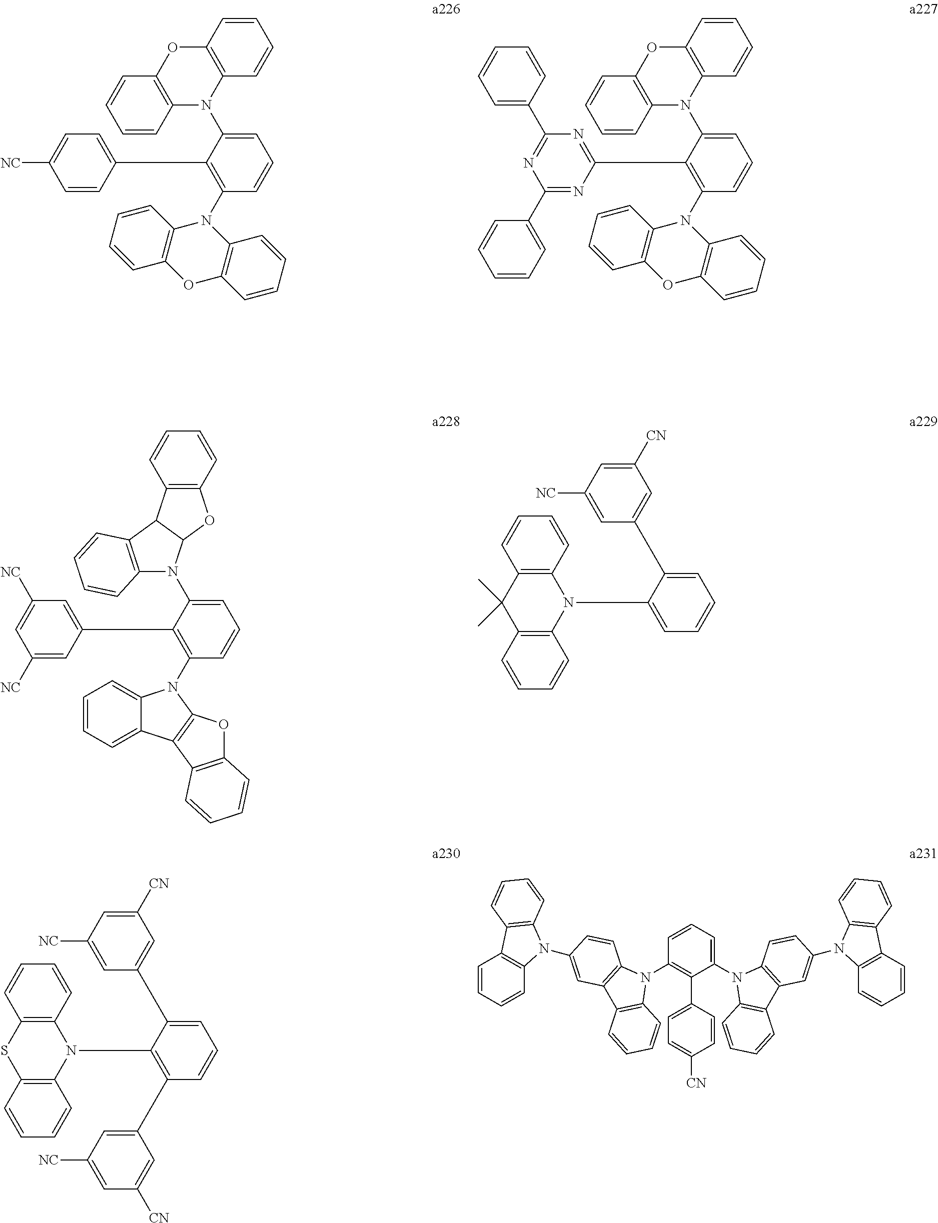 [SMILES: N#Cc1ccc(-c2c(N3c4ccccc4Oc4ccccc43)cccc2N2c3ccccc3Oc3ccccc32)cc1 ; c1ccc(-c2nc(-c3ccccc3)nc(-c3c(N4c5ccccc5Oc5ccccc54)cccc3N3c4ccccc4Oc4ccccc43)n2)cc1 ; N#Cc1cc(C#N)cc(-c2c(N3c4ccccc4C4c5ccccc5OC43)cccc2-n2c3ccccc3c3c4ccccc4oc32)c1 ; CC1(C)c2ccccc2N(c2ccccc2-c2cc(C#N)cc(C#N)c2)c2ccccc21 ; N#Cc1cc(C#N)cc(-c2cccc(-c3cc(C#N)cc(C#N)c3)c2N2c3ccccc3Sc3ccccc32)c1 ; N#Cc1ccc(-c2c(-n3c4ccccc4c4cc(-n5c6ccccc6c6ccccc65)ccc43)cccc2-n2c3ccccc3c3cc(-n4c5ccccc5c5ccccc54)ccc32)cc1]

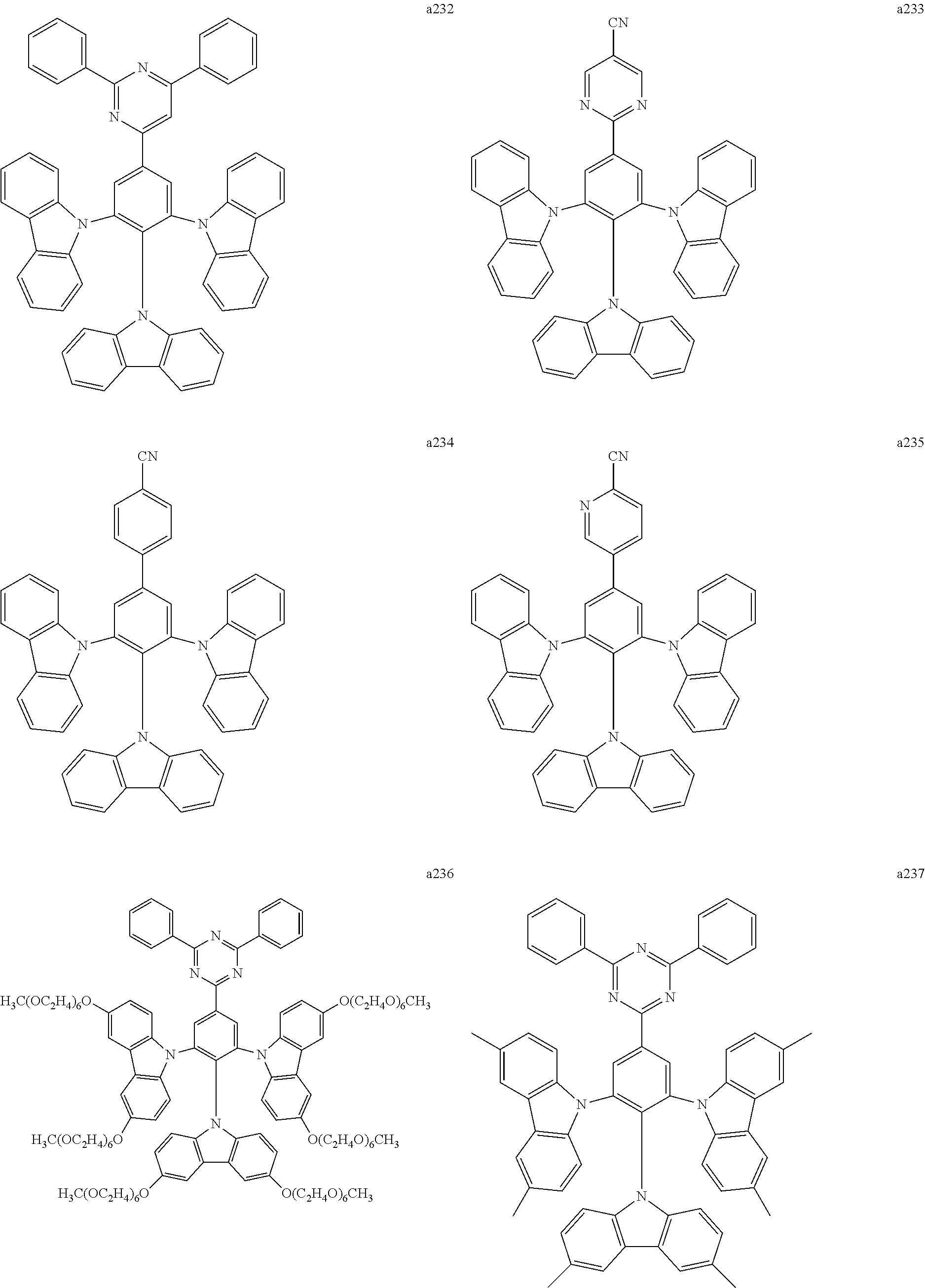

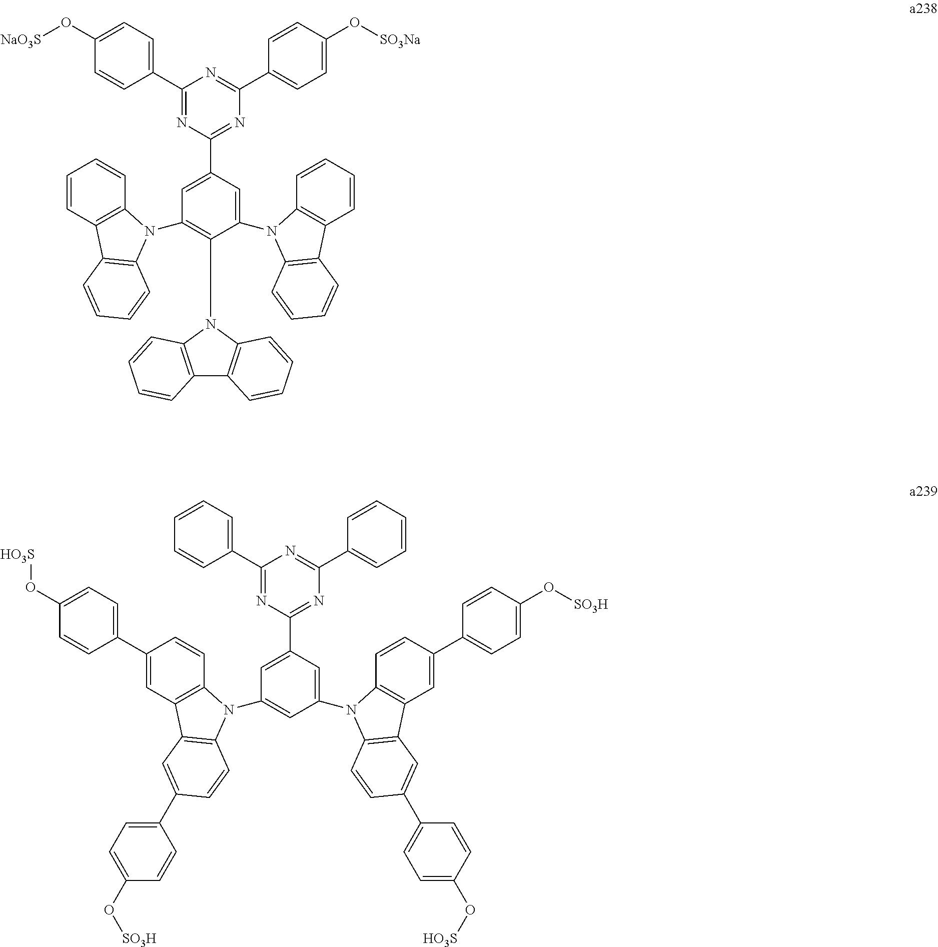

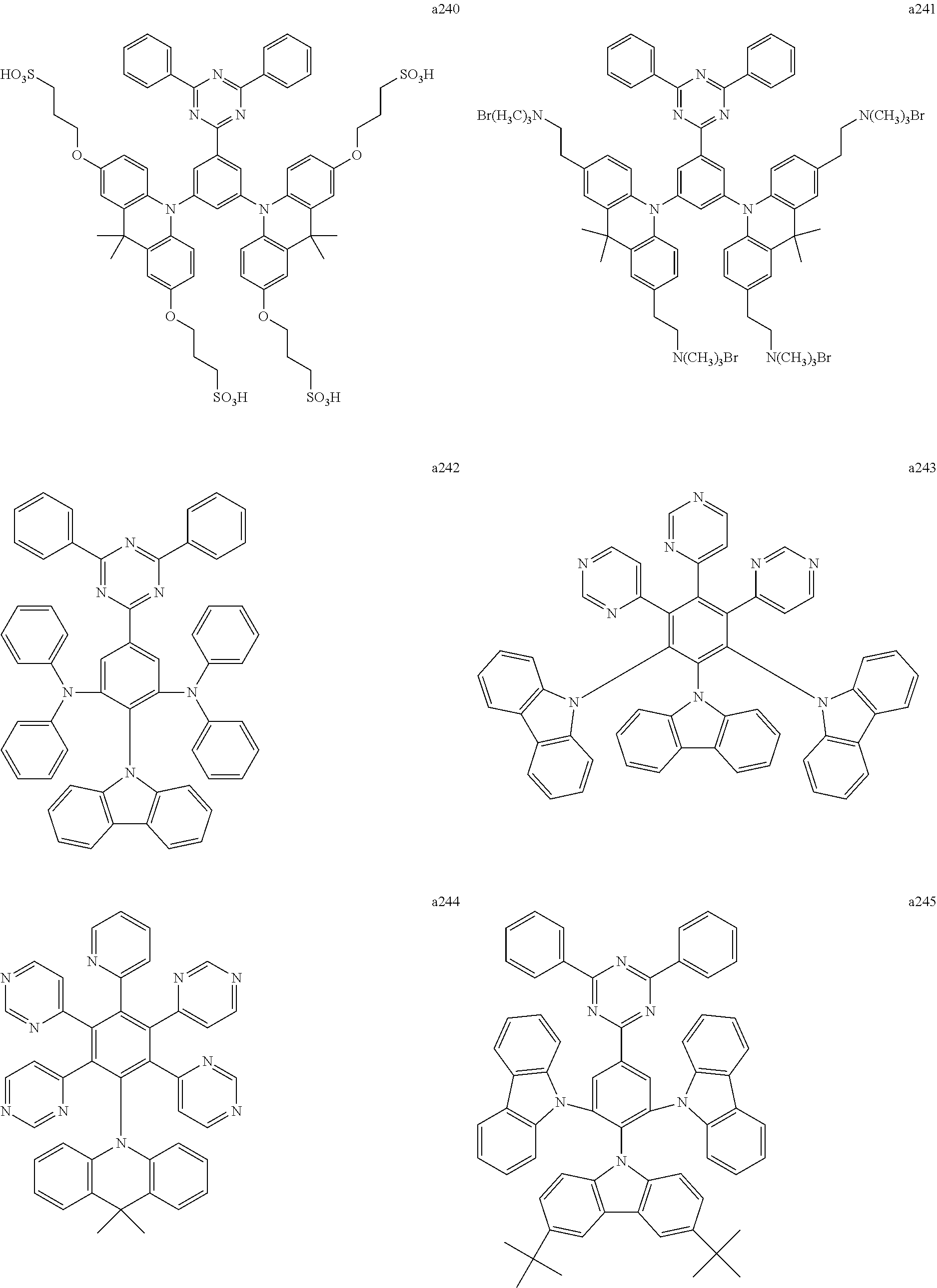

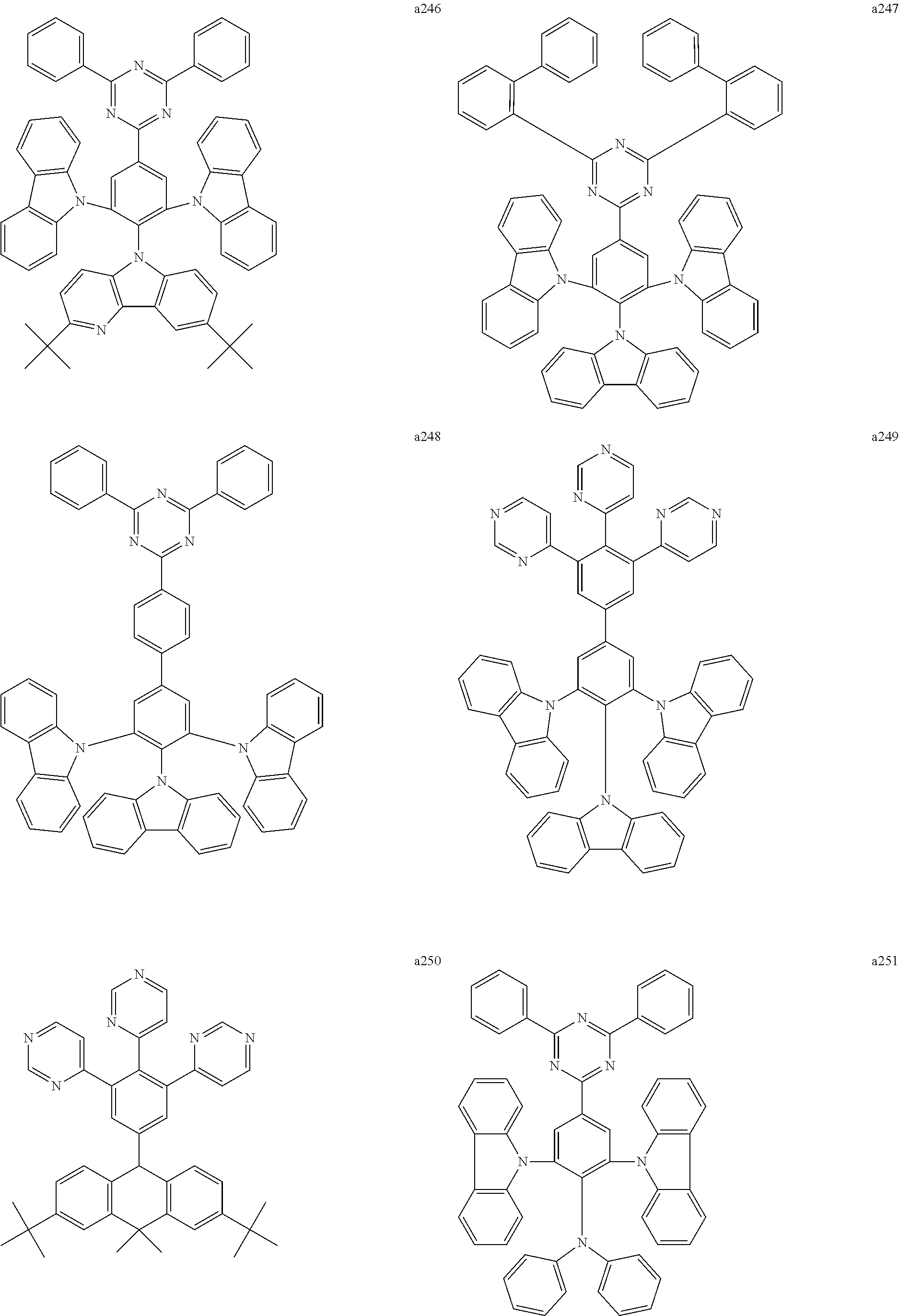

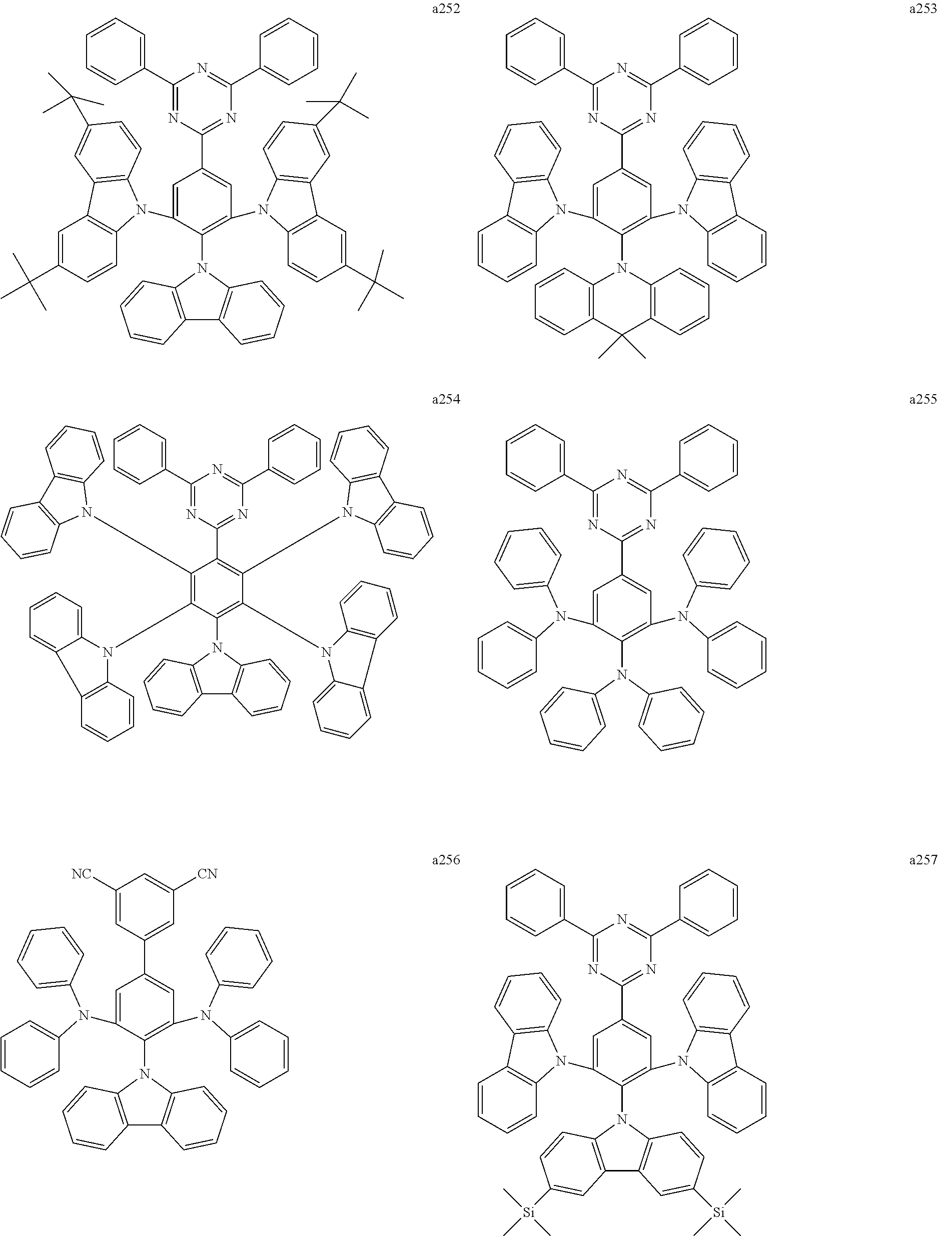

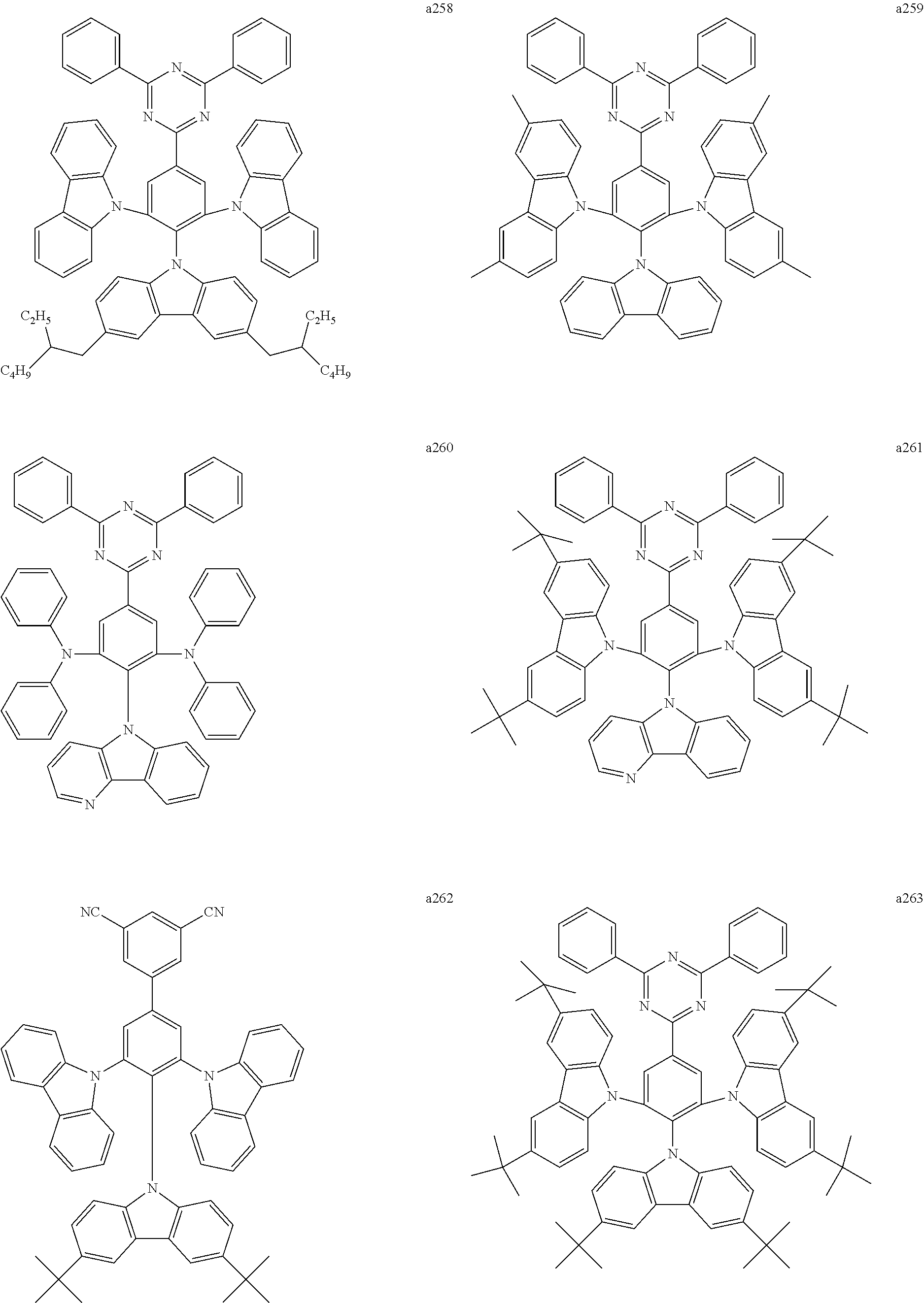

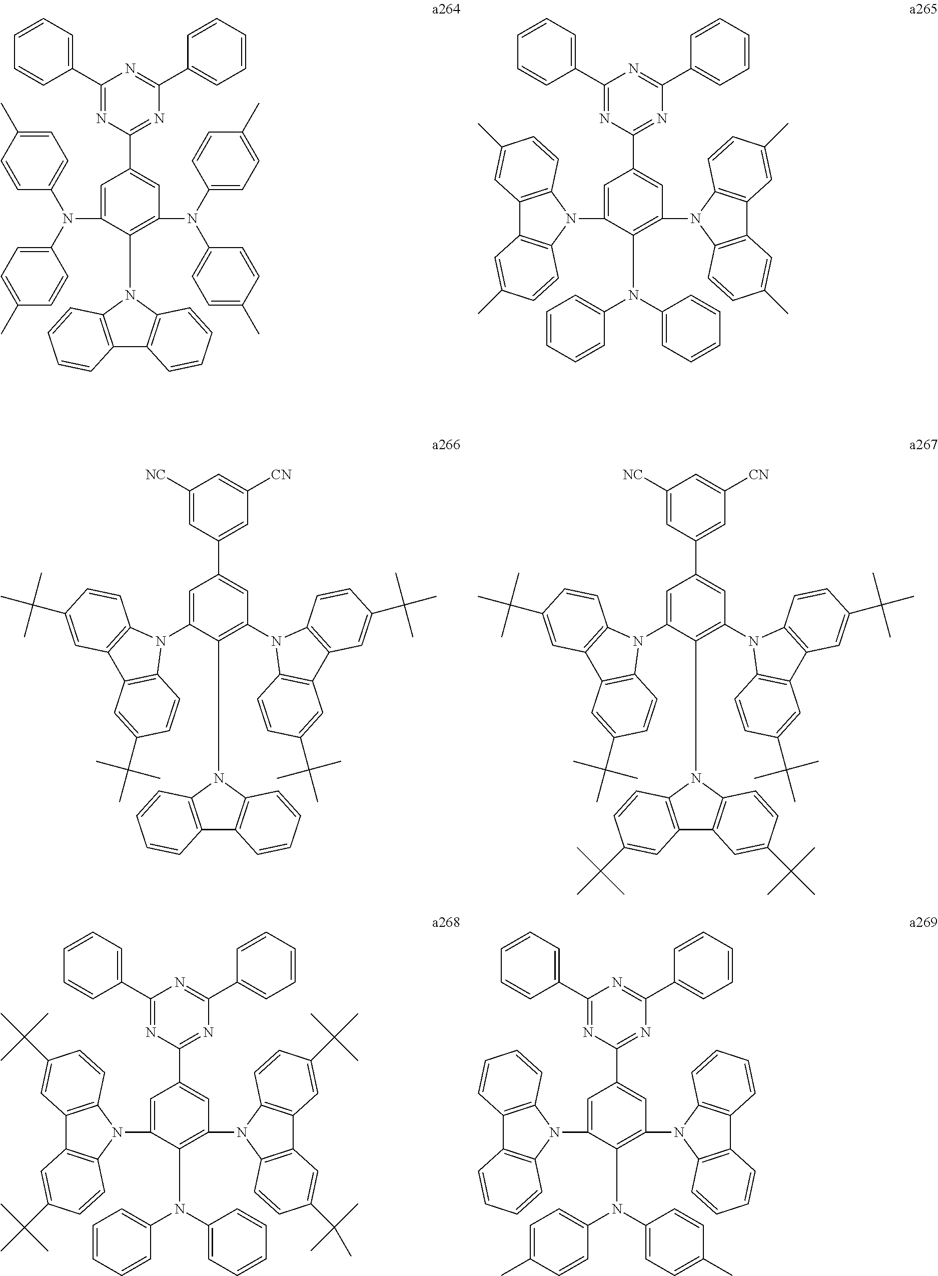

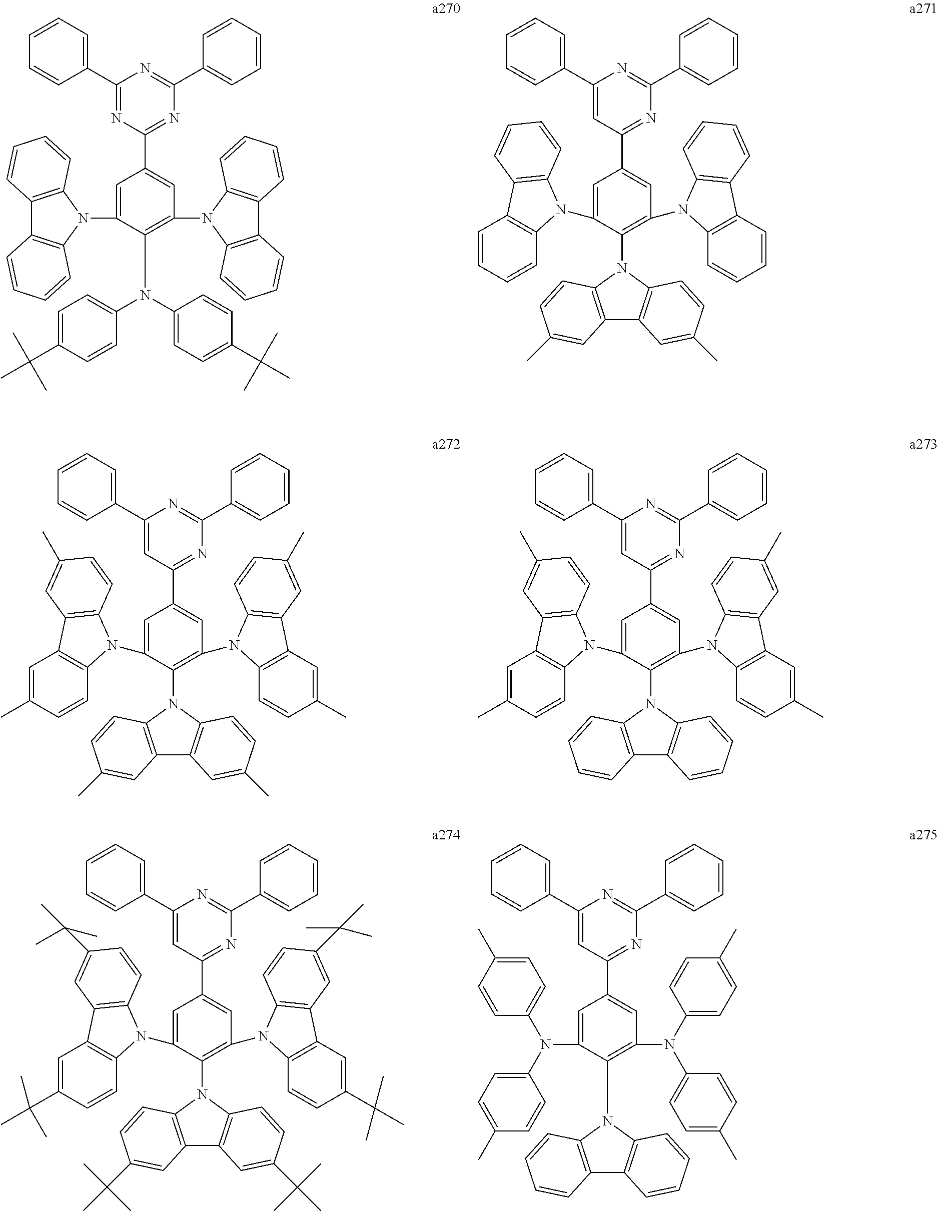

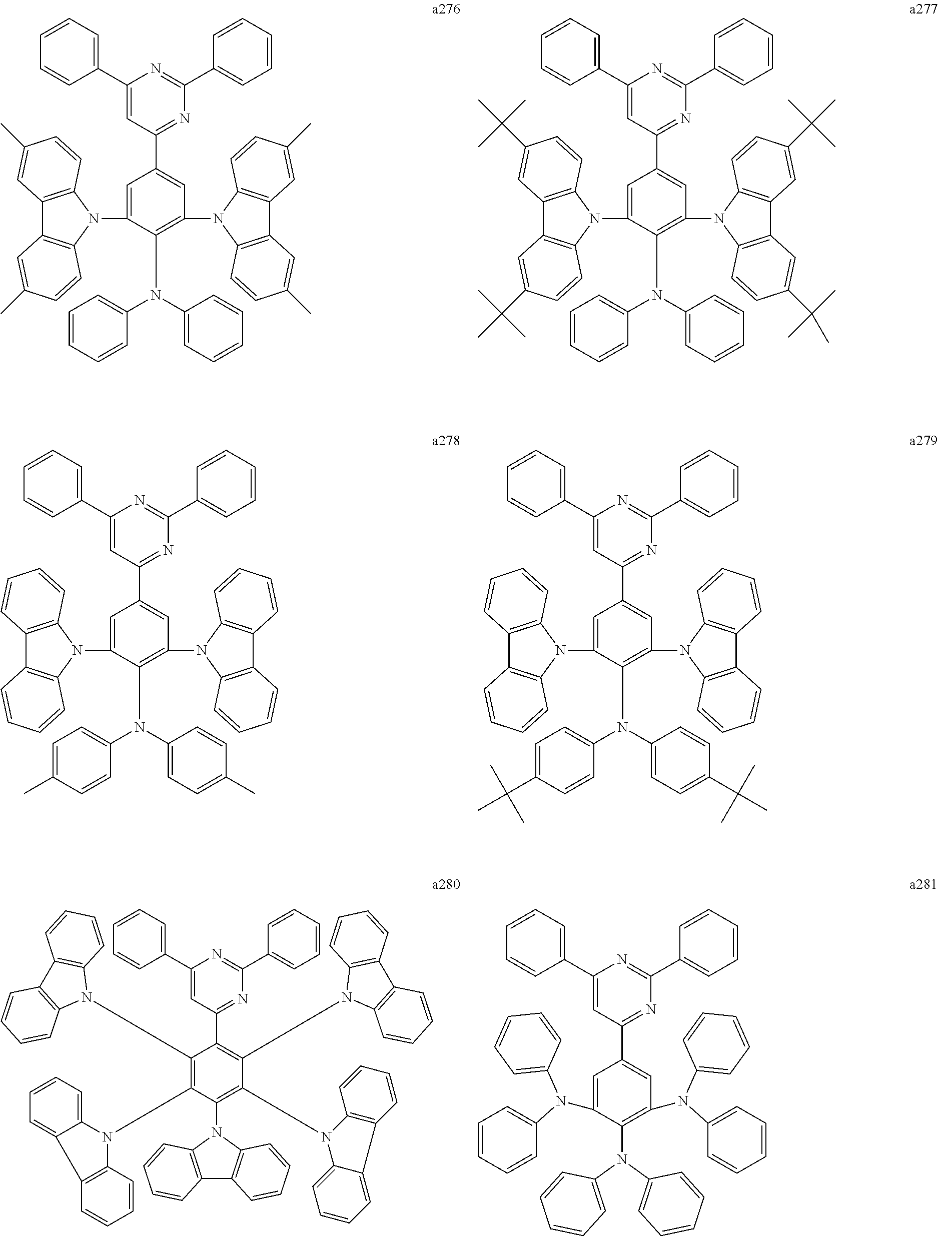

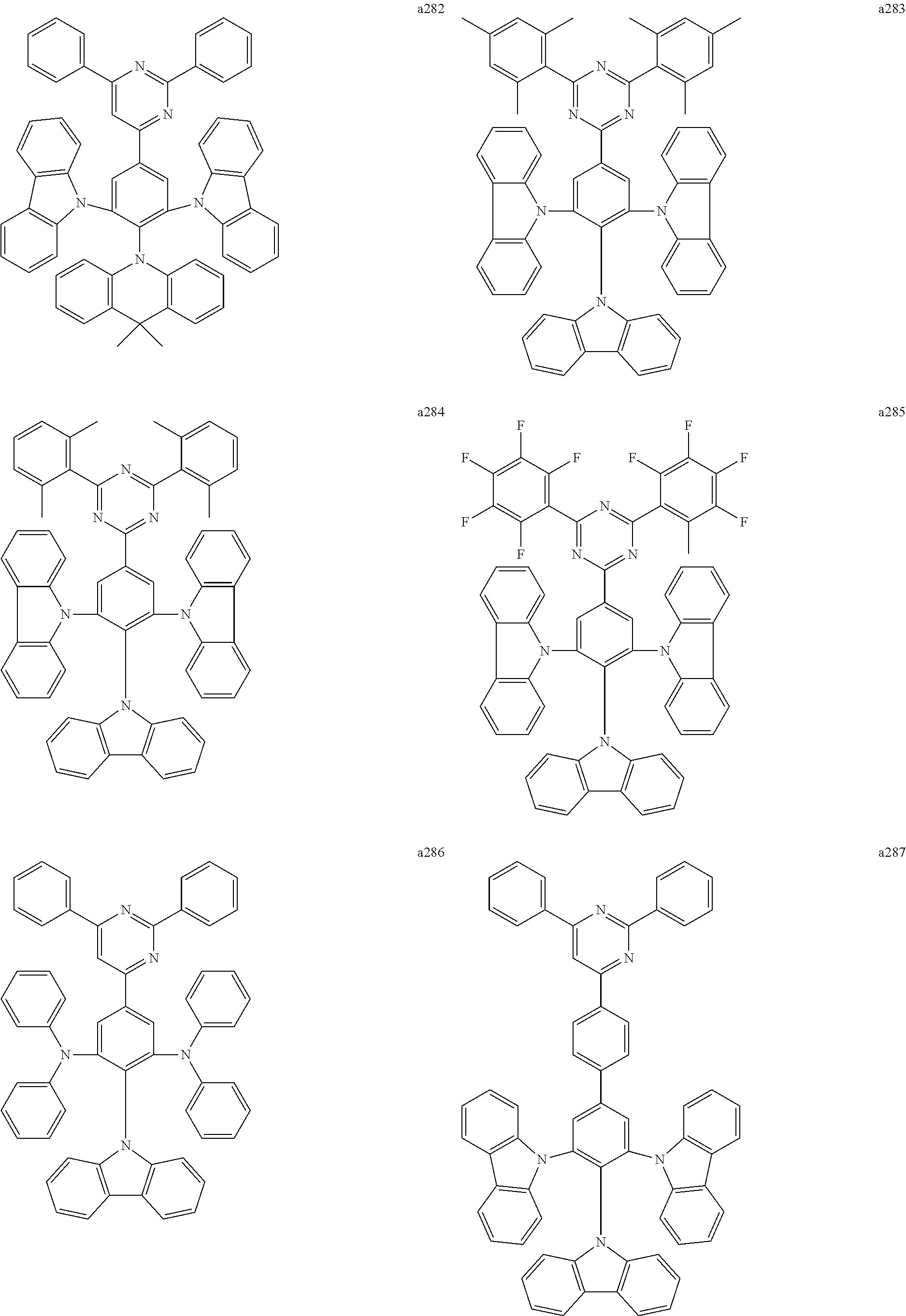

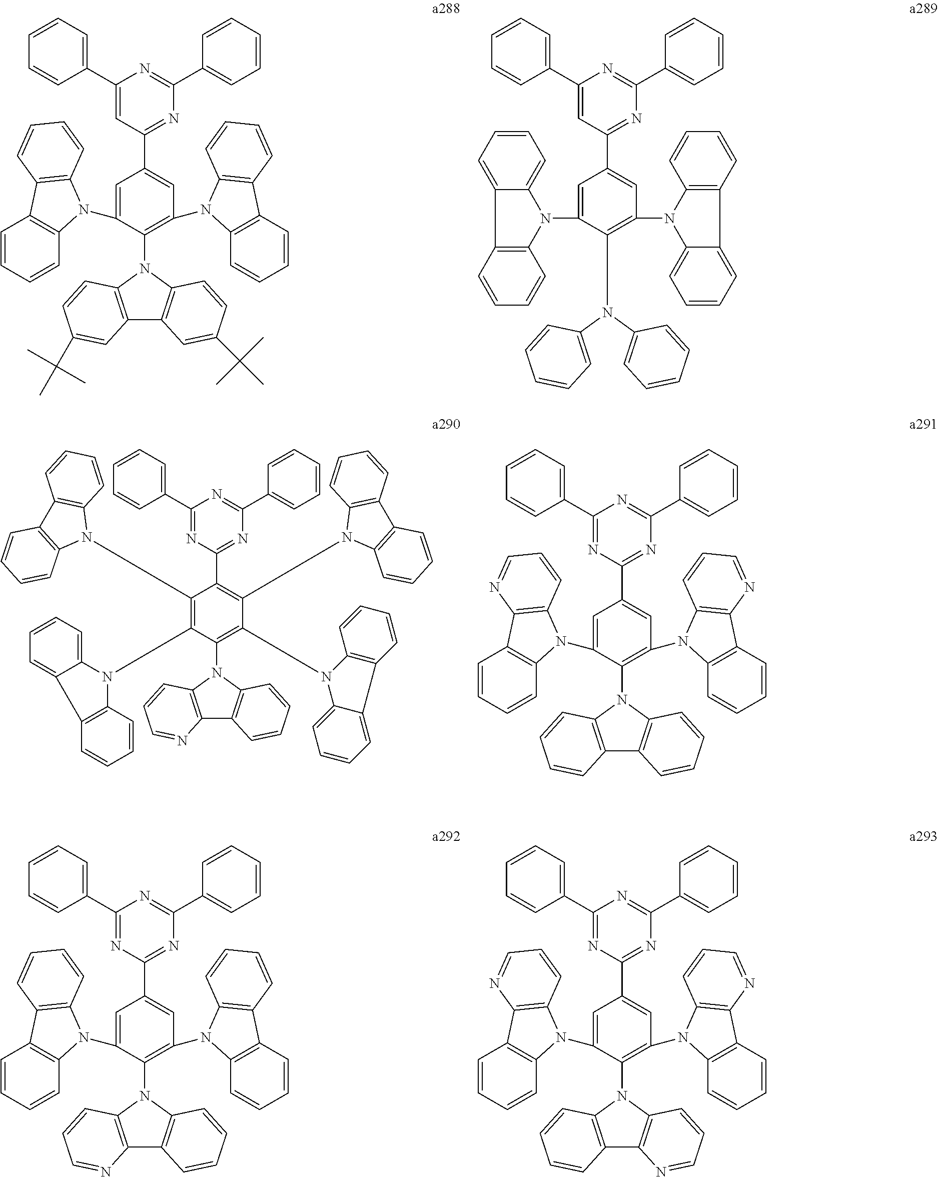

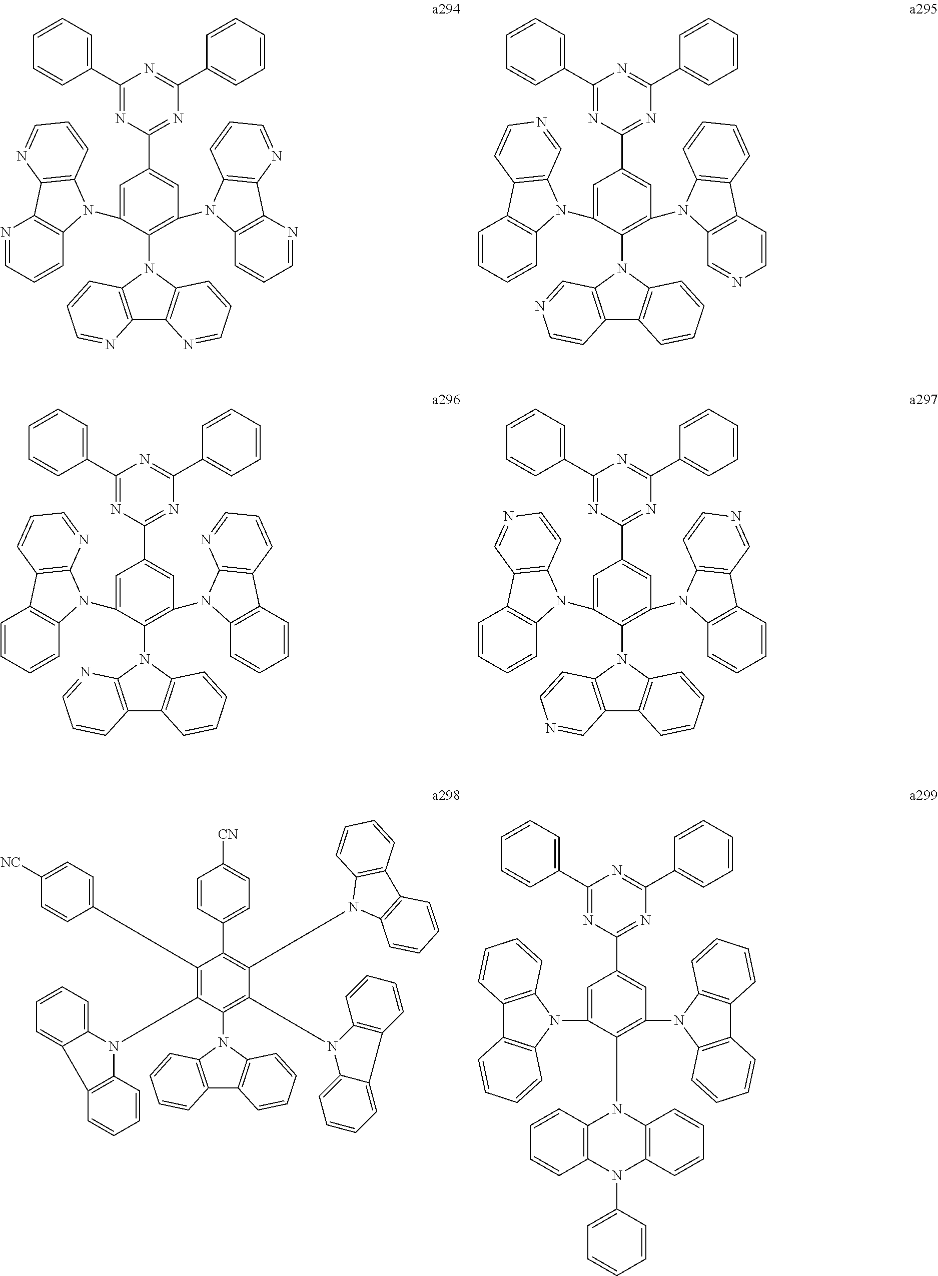

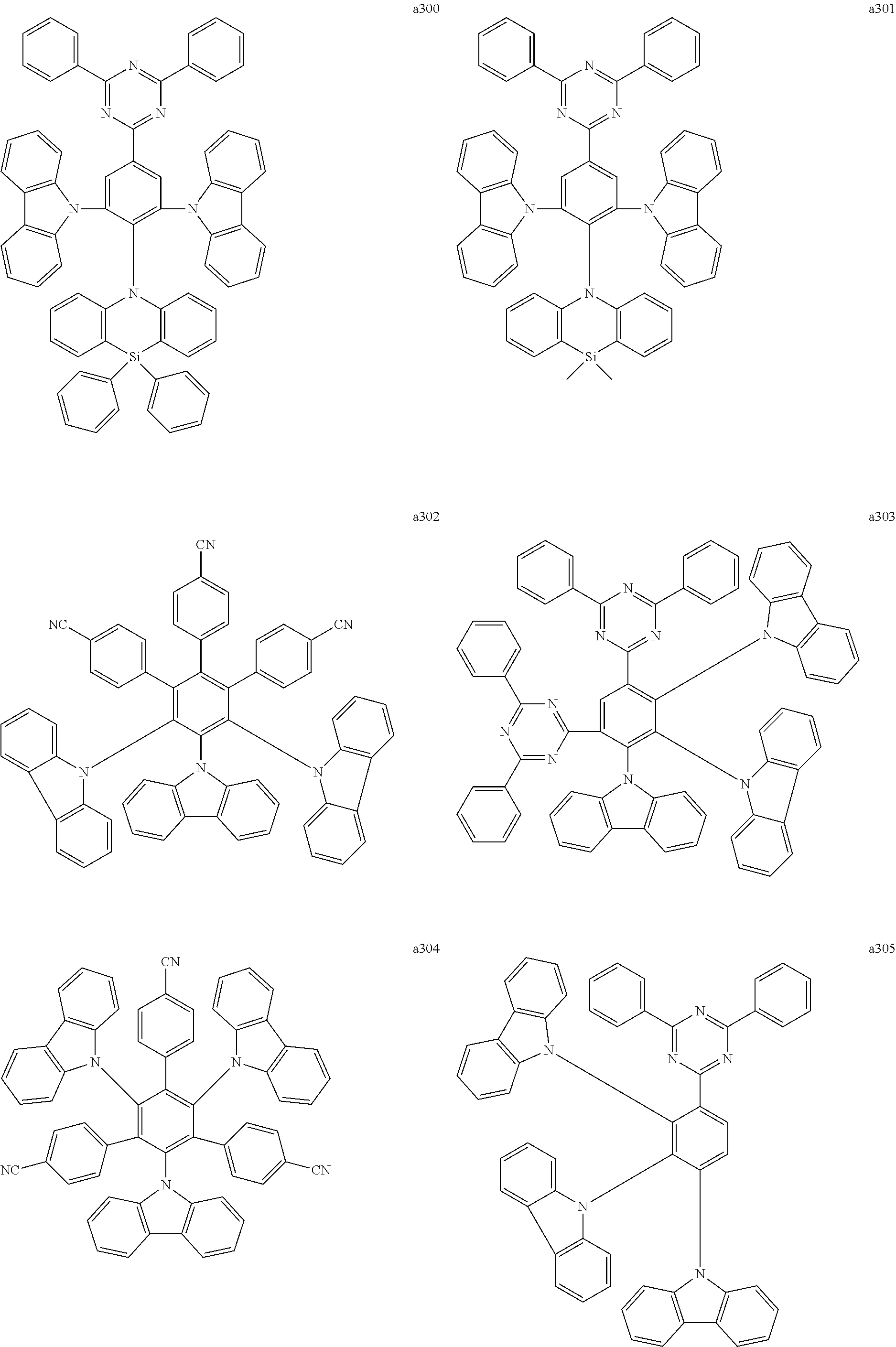

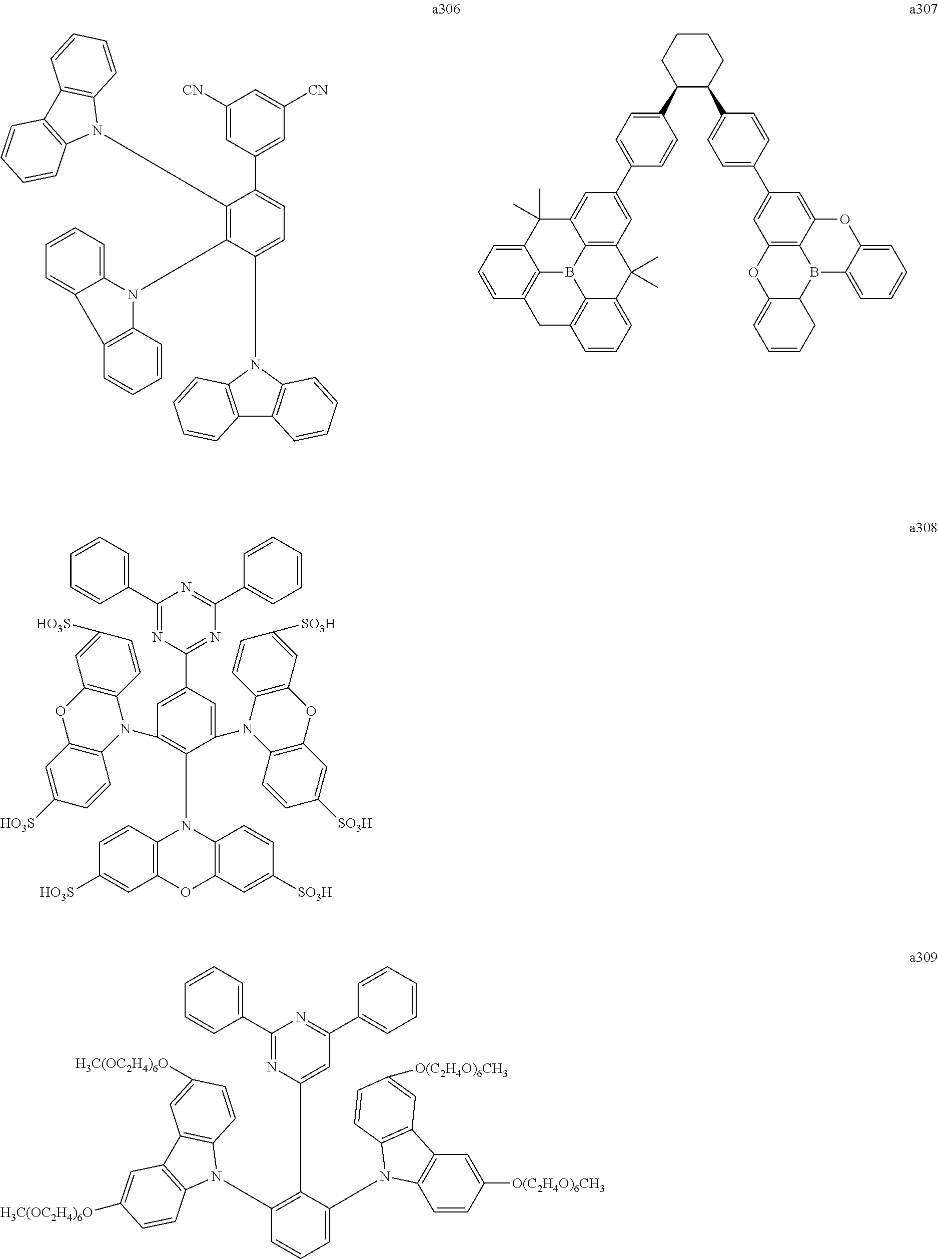

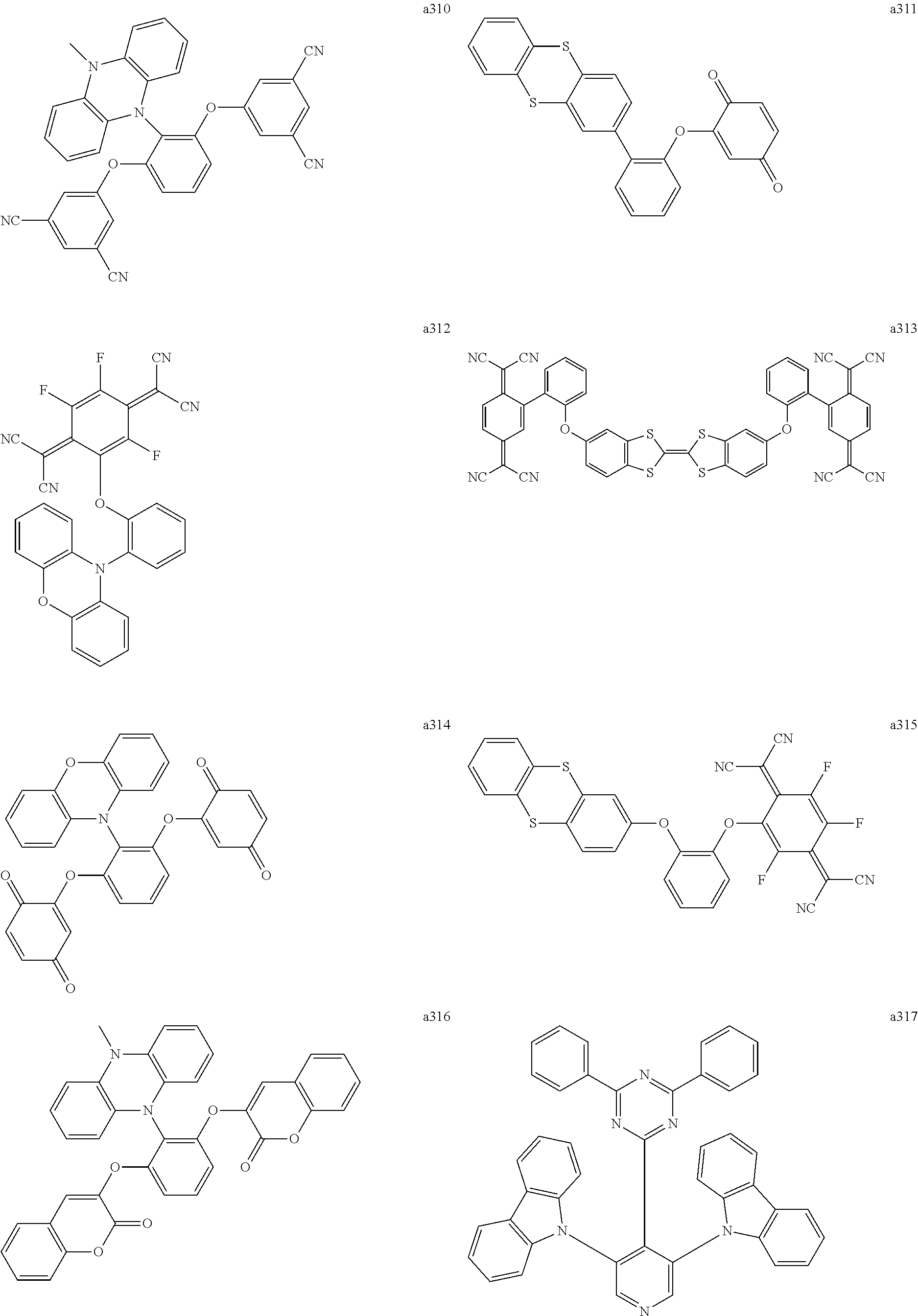

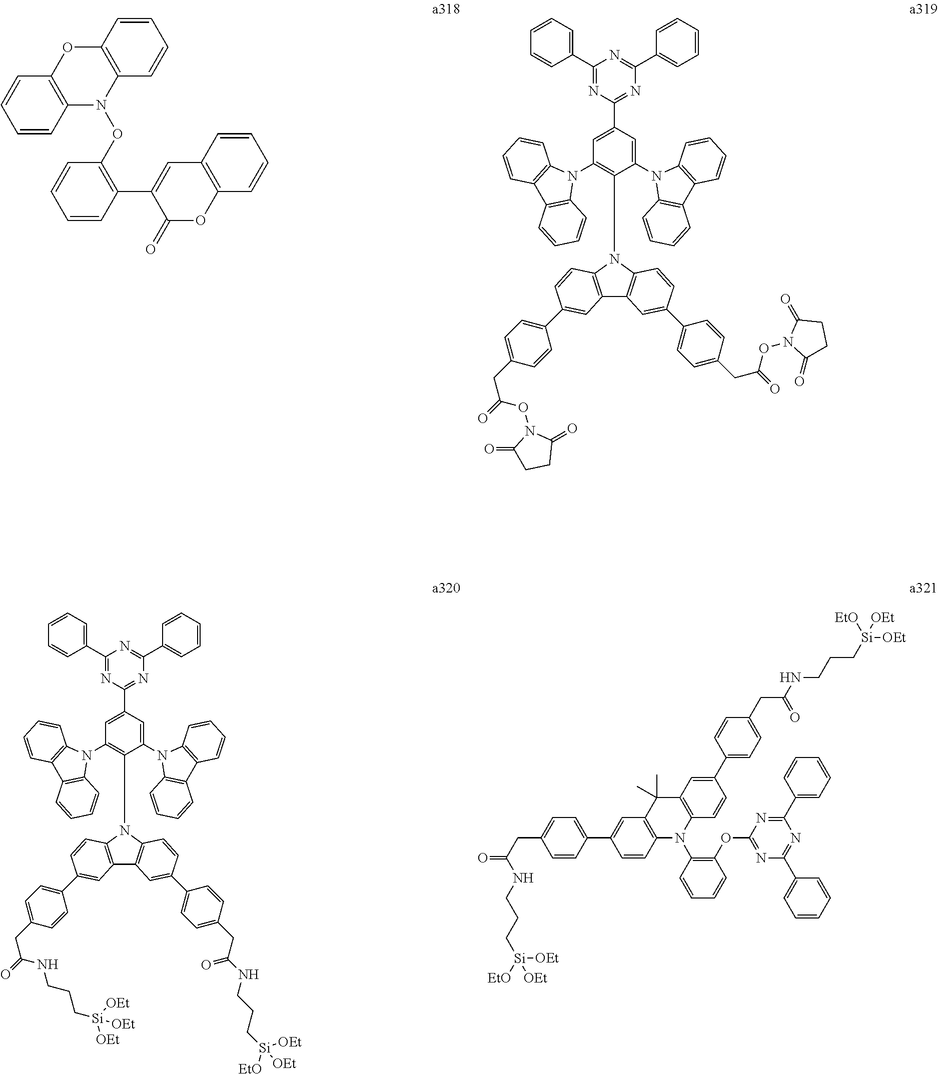

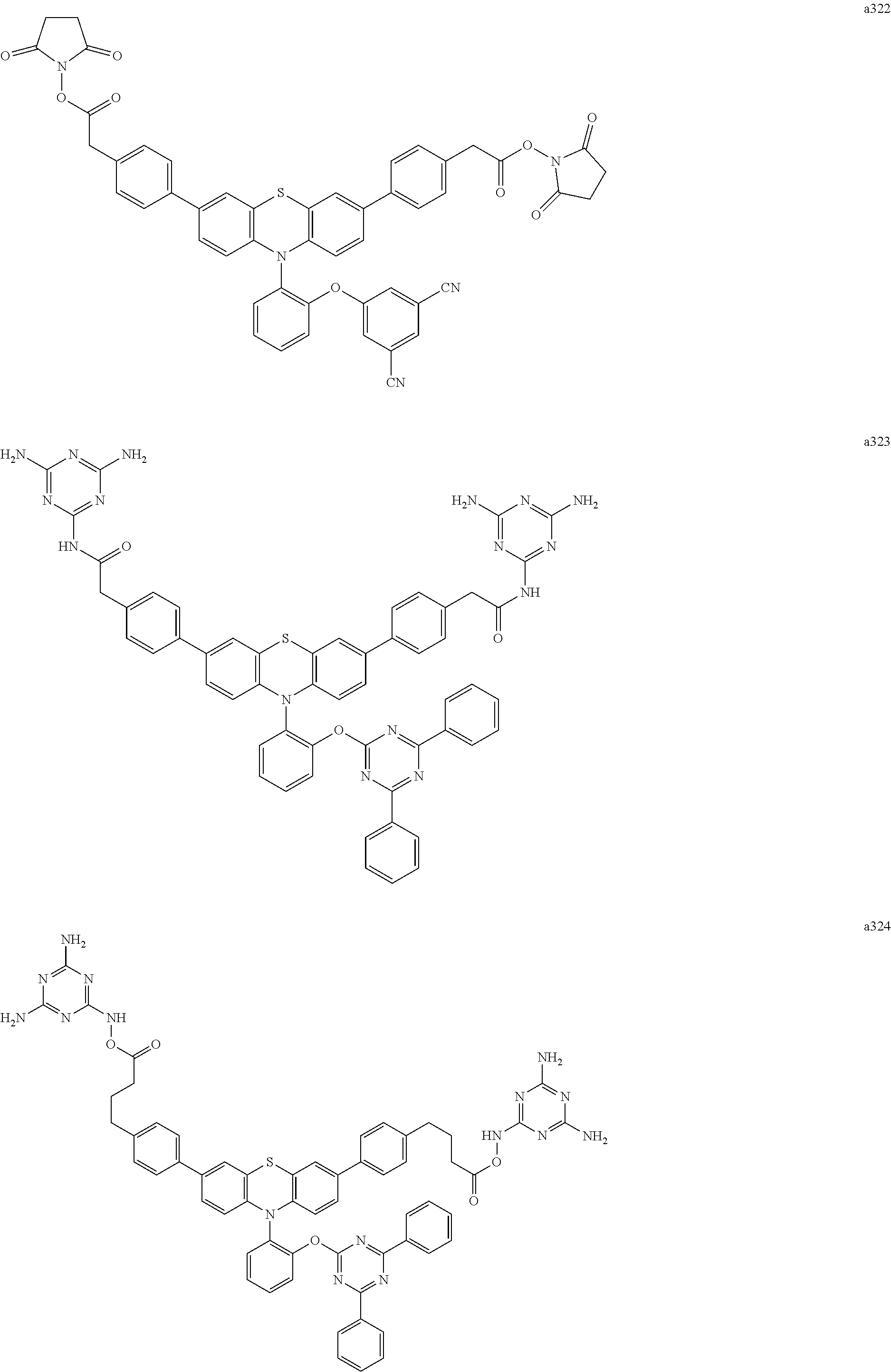 [SMILES: N#Cc1cc(C#N)cc(Oc2ccccc2N2c3ccc(-c4ccc(CC(=O)ON5C(=O)CCC5=O)cc4)cc3Sc3cc(-c4ccc(CC(=O)ON5C(=O)CCC5=O)cc4)ccc32)c1 ; Nc1nc(N)nc(NC(=O)Cc2ccc(-c3ccc4c(c3)Sc3cc(-c5ccc(CC(=O)Nc6nc(N)nc(N)n6)cc5)ccc3N4c3ccccc3Oc3nc(-c4ccccc4)nc(-c4ccccc4)n3)cc2)n1 ; Nc1nc(N)nc(NOC(=O)CCCc2ccc(-c3ccc4c(c3)Sc3cc(-c5ccc(CCCC(=O)ONc6nc(N)nc(N)n6)cc5)ccc3N4c3ccccc3Oc3nc(-c4ccccc4)nc(-c4ccccc4)n3)cc2)n1]

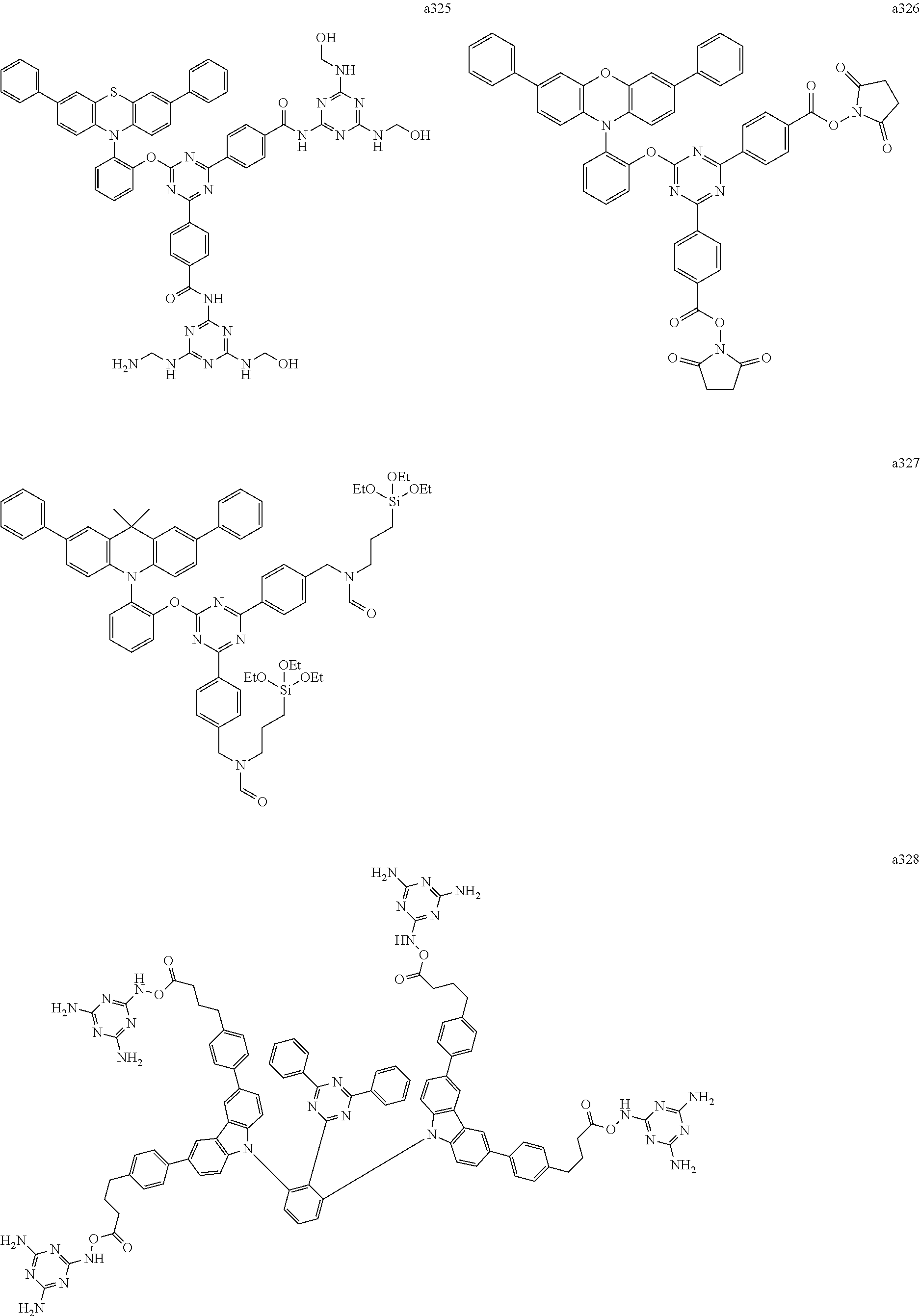

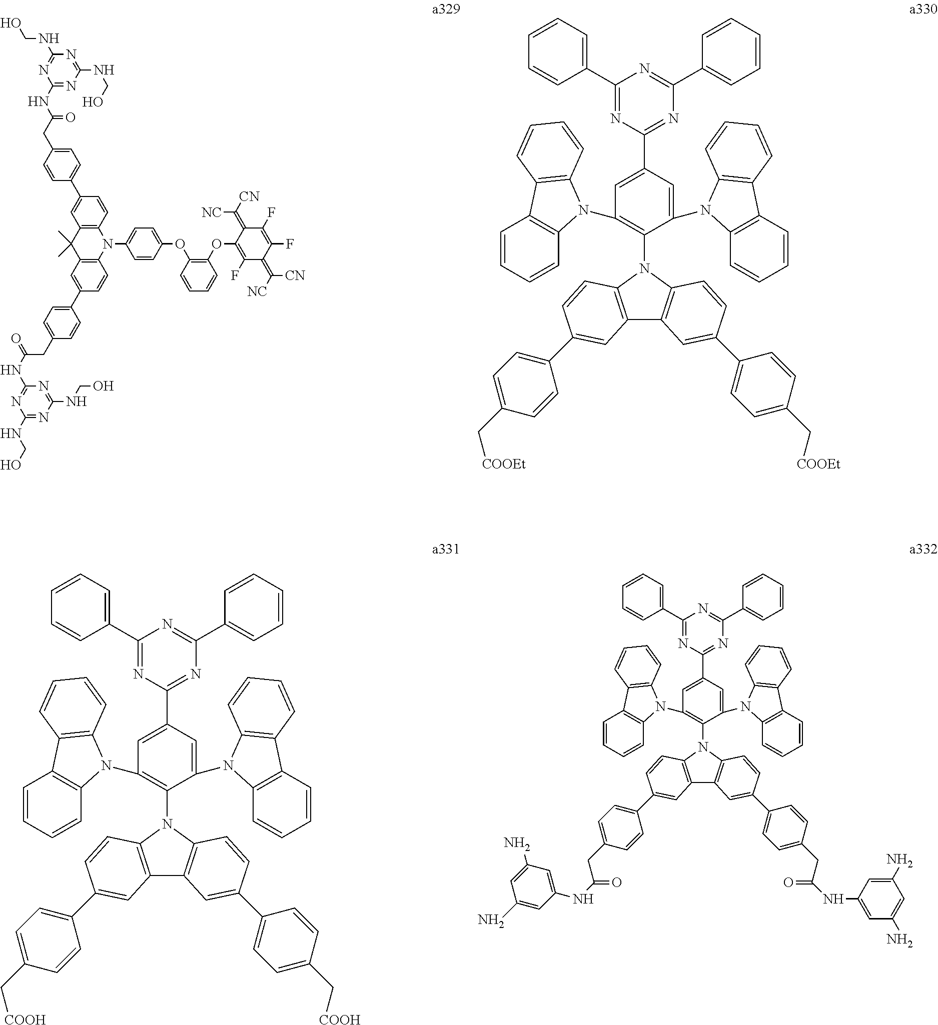

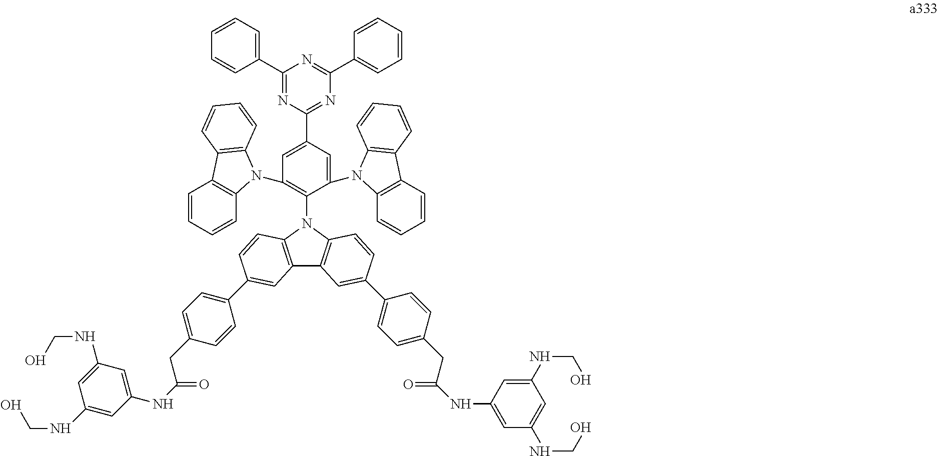 [SMILES: O=C(Cc1ccc(-c2ccc3c(c2)c2cc(-c4ccc(CC(=O)Nc5cc(NCO)cc(NCO)c5)cc4)ccc2n3-c2c(-n3c4ccccc4c4ccccc43)cc(-c3nc(-c4ccccc4)nc(-c4ccccc4)n3)cc2-n2c3ccccc3c3ccccc32)cc1)Nc1cc(NCO)cc(NCO)c1]

### <1.4> Production Method of Luminescent Dye

Compounds having the structure represented by Formula (1) may be synthesized, for example, by the method described in WO 2014/022008, or may be synthesized by referring to the methods described in the references.

### Synthesis Example

The synthesis flow of the compound having the structure represented by Formula (1) is shown below.

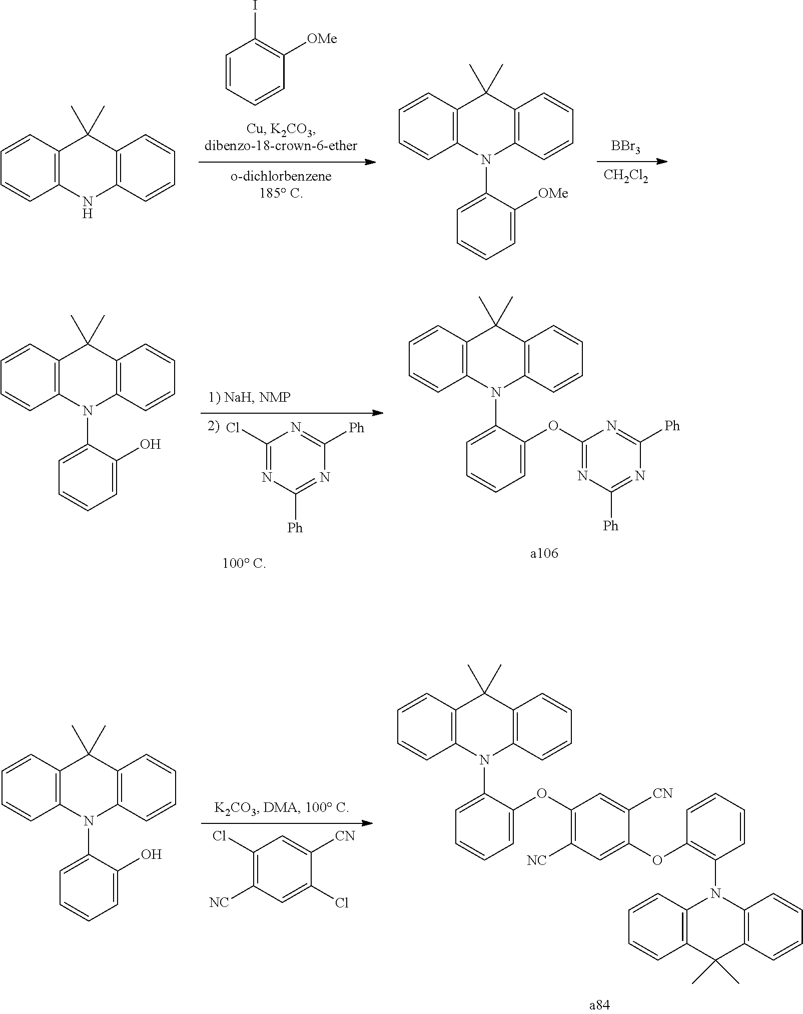

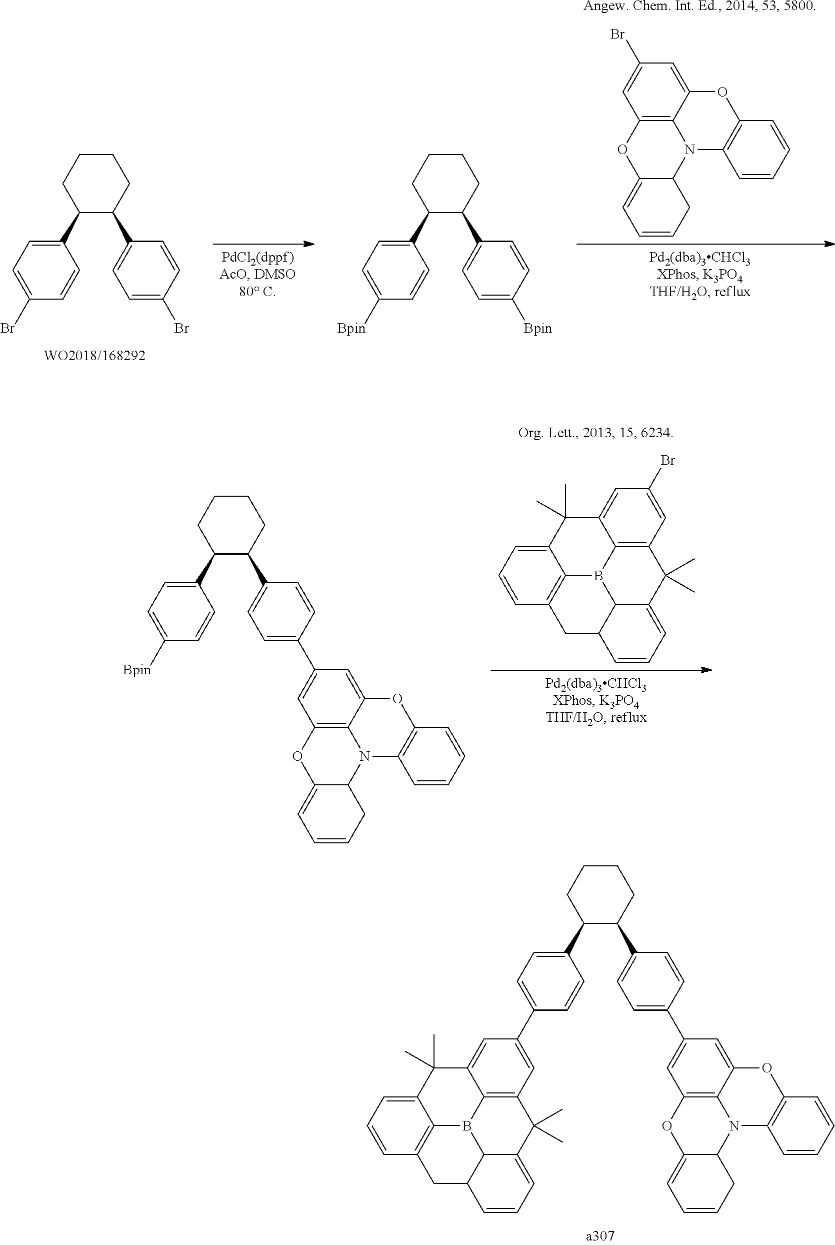

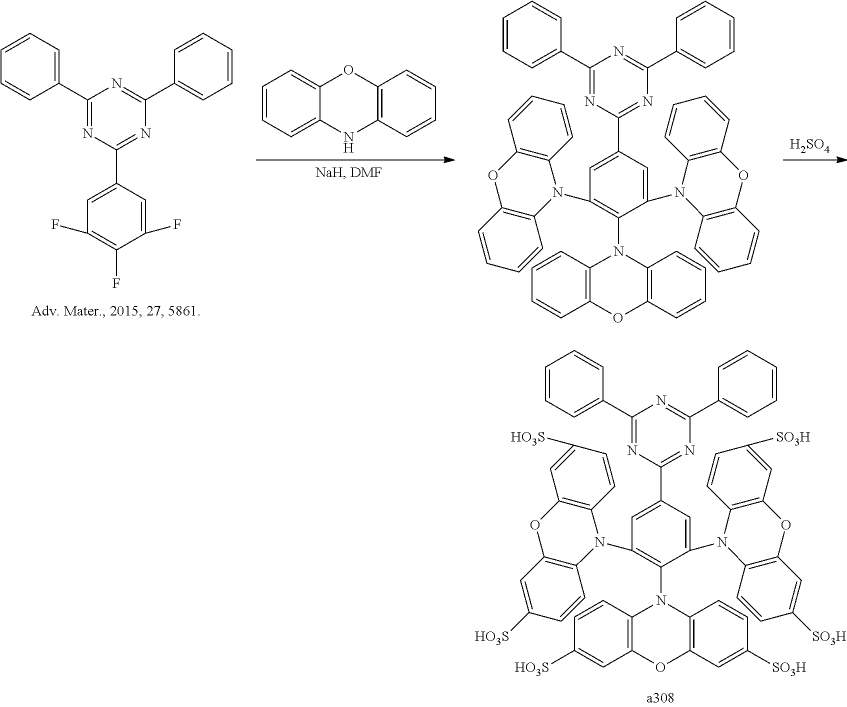

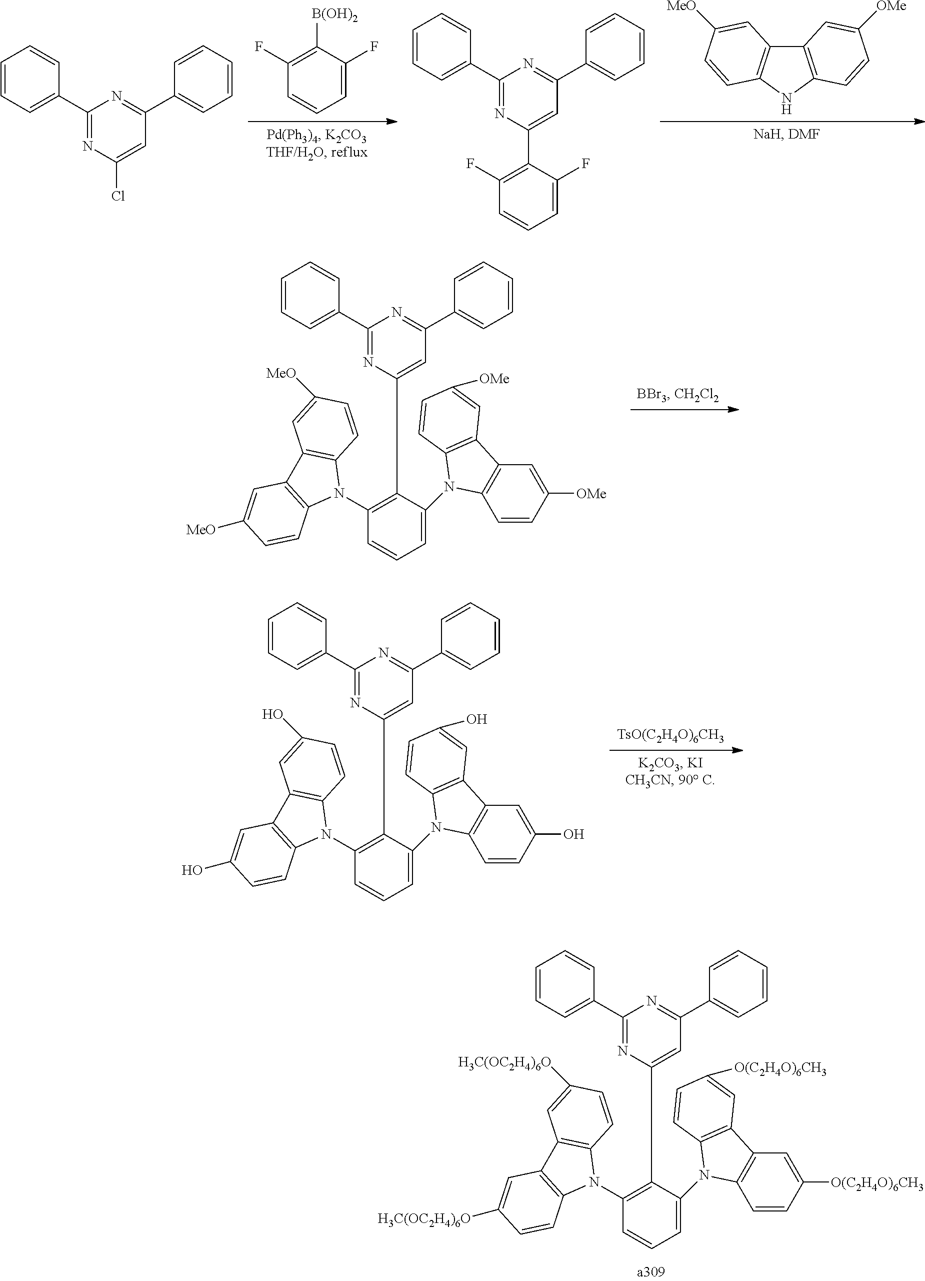

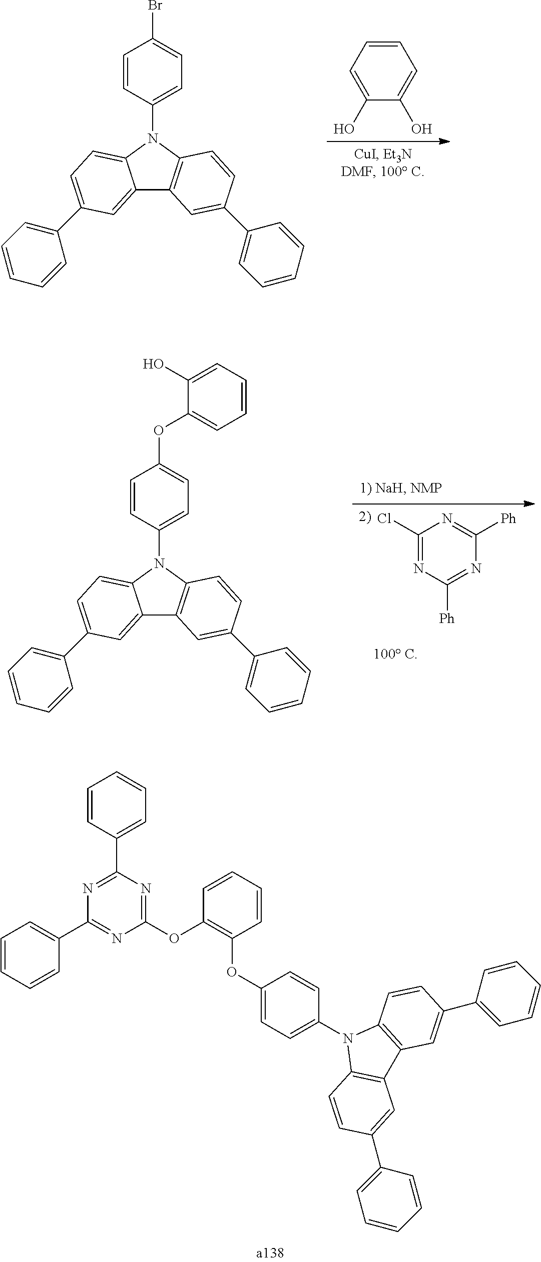

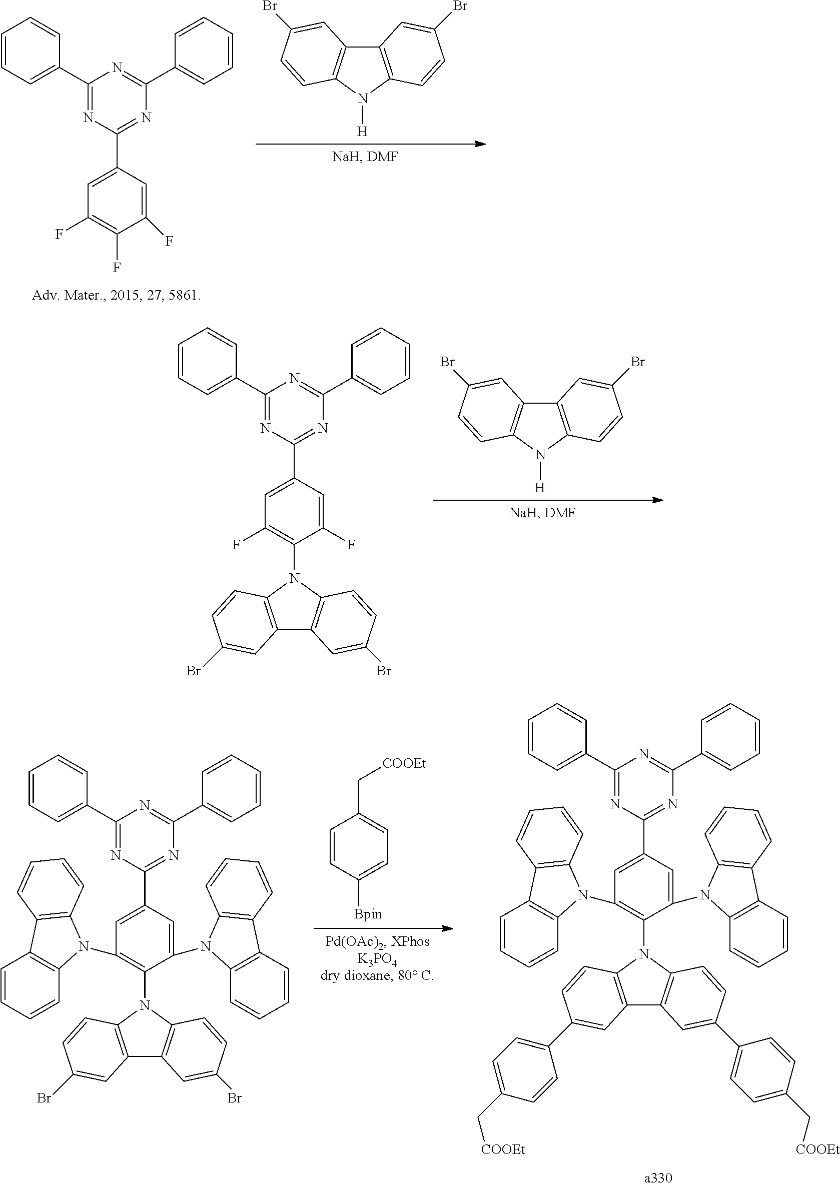

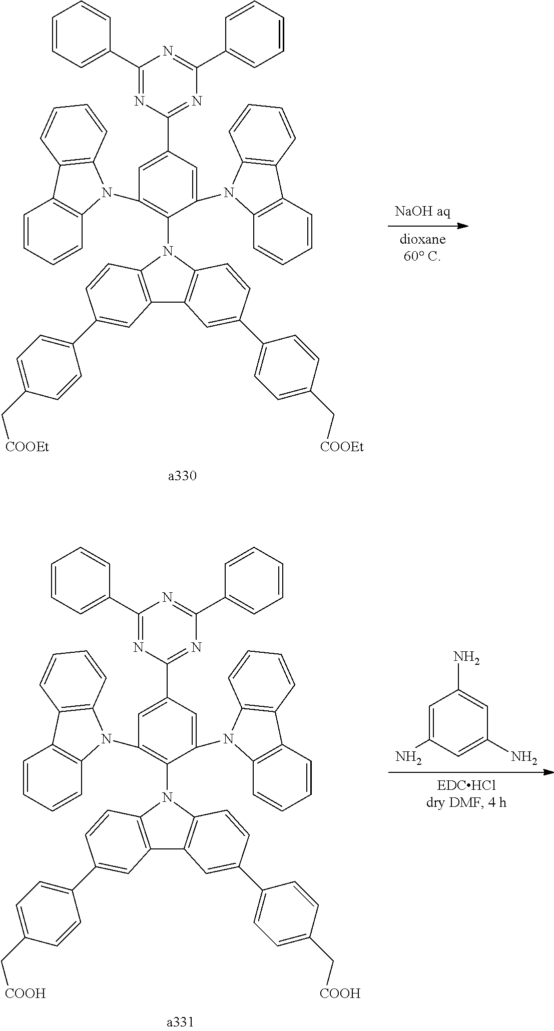

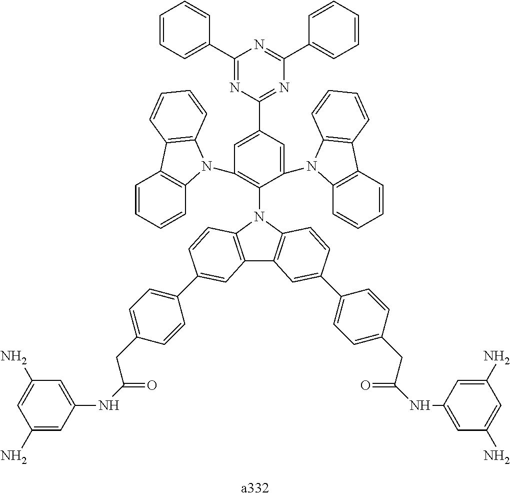 [SMILES: Nc1cc(N)cc(NC(=O)Cc2ccc(-c3ccc4c(c3)c3cc(-c5ccc(CC(=O)Nc6cc(N)cc(N)c6)cc5)ccc3n4-c3c(-n4c5ccccc5c5ccccc54)cc(-c4nc(-c5ccccc5)nc(-c5ccccc5)n4)cc3-n3c4ccccc4c4ccccc43)cc2)c1]

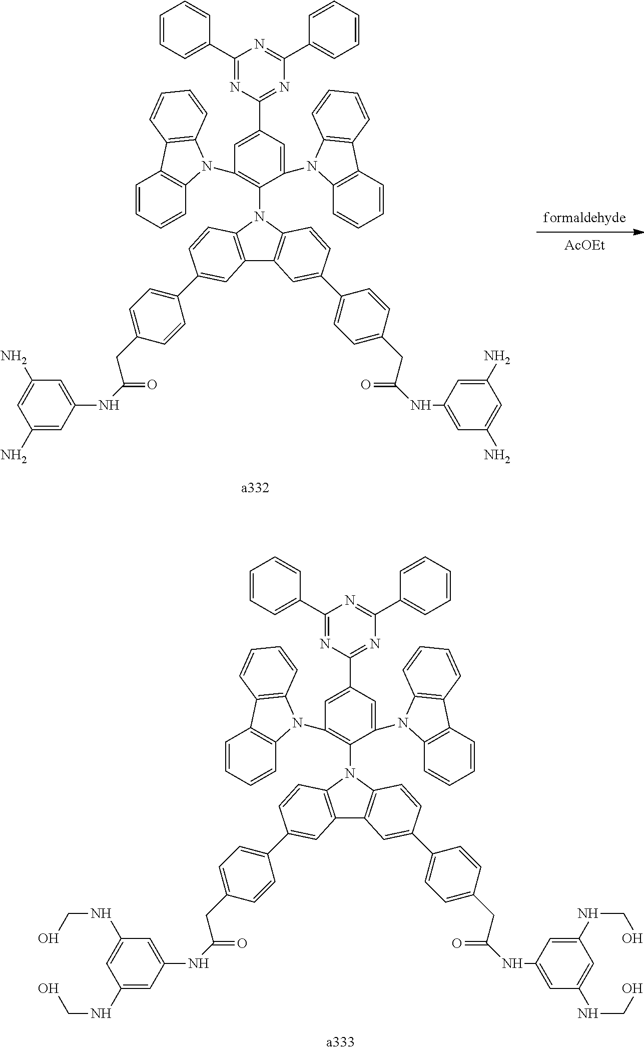 [SMILES: "AcOEt" ; Nc1cc(N)cc(NC(=O)Cc2ccc(-c3ccc4c(c3)c3cc(-c5ccc(CC(=O)Nc6cc(N)cc(N)c6)cc5)ccc3n4-c3c(-n4c5ccccc5c5ccccc54)cc(-c4nc(-c5ccccc5)nc(-c5ccccc5)n4)cc3-n3c4ccccc4c4ccccc43)cc2)c1 ; "formaldehyde" ; O=C(Cc1ccc(-c2ccc3c(c2)c2cc(-c4ccc(CC(=O)Nc5cc(NCO)cc(NCO)c5)cc4)ccc2n3-c2c(-n3c4ccccc4c4ccccc43)cc(-c3nc(-c4ccccc4)nc(-c4ccccc4)n3)cc2-n2c3ccccc3c3ccccc32)cc1)Nc1cc(NCO)cc(NCO)c1]

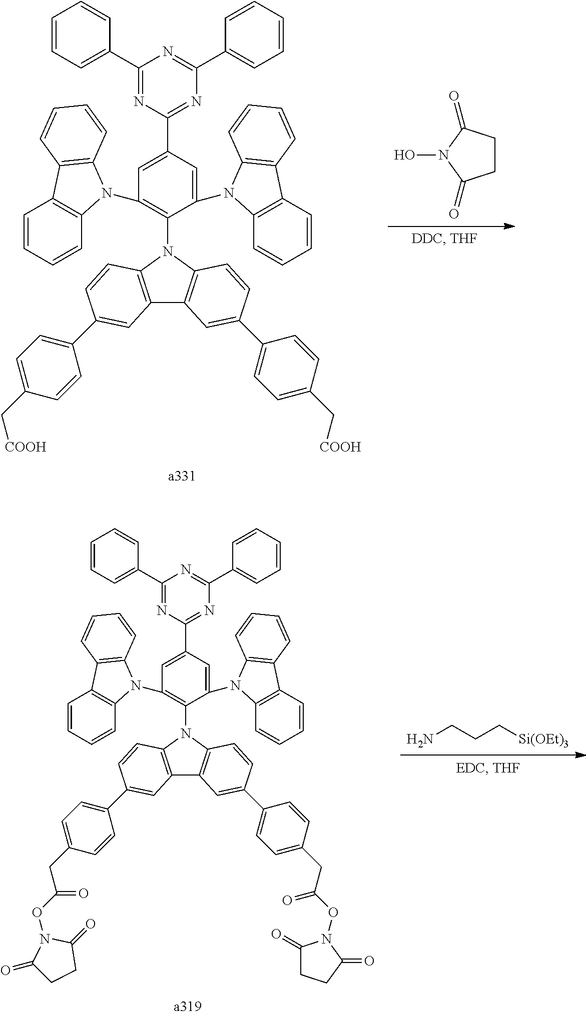

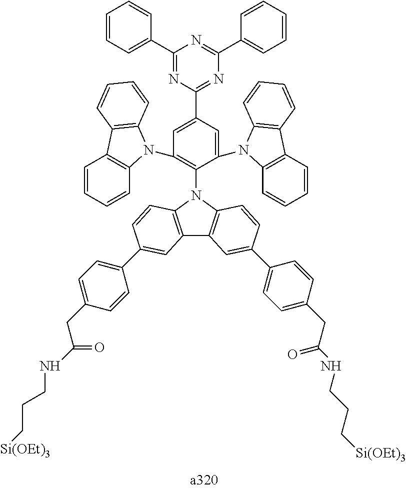

### <1.5> Binder

It is preferred that the luminescent nanoparticles of the present invention contain a binder that acts as a sticking material or binding material, in that a special function may be given to the particle surface via the binder.

When a binder is included, the luminescent dye contained in the luminescent nanoparticle is preferably in the range of 20 to 500 μmol to 1 g of the binder in terms of the effectiveness of the binder.

The binder is preferably an organic resin having a molecular weight of 300 or more containing carbon atoms in the main chain or a metal alkoxide. Specific examples of the organic resin include polyolefin resins such as polypropylene, polymethylpentene, and polycyclohexylenedimethylene terephthalate (PCT), polyamide, polyacetal, polyethylene terephthalate polyethylene terephthalate, polybutylene terephthalate, polyphenylene sulfide, polycarbonate, ABS resin, AS resin, acrylic resin, amino resin, polyester resin, epoxy resin, mixed resin of acrylic resin and amino resin, mixed resin of polyester resin and amino resin, cellulose resin, polyethylene resin, polypropylene resin, polystyrene resin, polyvinyl chloride resin, polymethyl methacrylate resin, polyacrylonitrile resin, polyacrylamide resin, polyalcohol resin, polyallyl acetate resin, polyoxymethylene resin, poly-n-butyl isocyanate resin, polyethylene oxide resin, 6-nylon resin, poly-p-oxypropionate ester resin, phenol resin, urea resin, melamine resin, alkyd melamine resin, unsaturated polyester resin, polyvinyl alcohol resin, poly(N-vinylformamide) resin, poly(N-vinylisobutyramide) resin, polyacrylic acid resin, poly (N-isopropylacrylamide) resin, poly(N-vinylpyrrolidinone) resin, polyhydroxyethyl methacrylate resin, polyoxyethylene methacrylate resin, polyethylene glycol dimethyl ether resin, and polystyrene sulfonate resin. Specific examples of the metal of the metal alkoxide include magnesium, calcium, strontium, scandium, yttrium, ruthenium, lawrencium, lanthanum, titanium, zirconium, hafnium, cerium, vanadium, niobium, tantalum, chromium, molybdenum, tungsten manganese, ruthenium, cobalt, rhodium, iridium, nickel, platinum, palladium, copper, silver, gold, zinc, aluminum, gallium, indium, silicon, germanium, and tin.

(Binder with a Hydrophilic Group)

It is preferred that the binder on the surface of the luminescent nanoparticles of the present invention has a hydrophilic group because the particles may be dispersed in water by inhibiting aggregation between particles. The “hydrophilic group” refers to an atomic group that interacts strongly with water, and specific examples thereof include —OH, —SH, —COOH, —C(═O)H, —S(═O)₂OH, —S(═O)NH₂, —S(═O)₂NH₂, —P(═O)(OH)₃, —P(═O)R(OH)₂, —P(═O)R₂(OH), —P(OH)₃, —P(═O)(NH₂)₃, —P(═O)R(NH₂)₂, —P(═O)R₂(NH₂), —P(NH₂)₃, —O(C═O)OH, —NH₂, —NHR, —NHCONH₂, —NHCONHR, —NHCOOH, —Si(OH)₃, —Si(R)(OH)₂, —Si(R)₂OH, —Ge(OH)₃, —Ge(R)(OH)₂, —Ge(R)₂(OH), —Ti(OH)₃, —Ti(R)(OH)₂, —Si(NH₂)₃, —Si(R)(NH₂)₂, —B(OH)₂, —O—B(OH)₂, —O—B(OH)₂, —B(NH₂)₂, —NHB(OH)₂, and a polyethylene glycol group. The aforementioned R independently indicates a hydrogen atom or an alkyl group having a carbon number of 1 to 20. In addition, an NHS group and a maleimide group may also be mentioned as a hydrophilic group.

Specific examples of the binder having a hydrophilic group include an urea resin, a melamine resin, a polyvinyl alcohol resin, a poly(N-vinylformamide) resin, a poly(N-vinylisobutyramide) resin, a polyacrylic acid resin, a polyacrylamide resin, a poly(N-isopropylacrylamide) resin, a poly(N-vinylpyrrolidinone) resin, a polyhydroxyethyl methacrylate resin, a polyoxyethylene methacrylate resin, a polyethylene glycol dimethyl ether resin, a polystyrene sulfonate resin, titanium alkoxide, zirconium alkoxide, and silicon alkoxide.

(Covalent Bond with Luminescent Dye)

It is preferred that the luminescent nanoparticles of the present invention form a covalent bond between the binder and the luminescent dye contained in order to prevent leakage of the luminescent dye from the particles. For the luminescent nanoparticles of the present invention to form a covalent bond between the binder and the luminescent dye contained in the luminescent nanoparticles, it is preferred that the luminescent dye has a polymerized group, such as a melamine group, a methylolmelamine group, a trialkoxysilyl group, which reacts with the organic resin or metal alkoxide that serves as the binder described above, for example.

### (Thermosetting Resin)

The binder of the present invention may be a thermosetting resin. For example, from the viewpoint that the luminescent dye is hardly eluted in the clearing process using an organic solvent such as xylene, a thermosetting resin such as a melamine resin is preferred, which can immobilize the luminescent dye inside a dense crosslink structure.

The thermosetting resin includes, for example, a constituent unit formed from at least one monomer selected from the group consisting of melamine, urea, guanamines (including benzoguanamine, and acetoguanamine) and derivatives thereof. Any one of these monomers may be used alone or in combination with two or more monomers. If desired, one or more comonomers other than the above compounds may be further used in combination.

Examples of thermosetting resins include a melamine-formaldehyde resin and a urea-formaldehyde resin.

As raw materials for these thermosetting resins, not only the monomers themselves as described above but also prepolymers obtained by reacting the monomers with compounds such as formaldehyde and other crosslinking agents in advance may be used. For example, in the manufacture of a melamine formaldehyde resin, methylolmelamine prepared by condensing melamine and formaldehyde under alkaline conditions is generally used as a prepolymer. The compound may be further alkyl etherified (methylated to improve stability in water, or butylated to improve solubility in organic solvents).

The thermosetting resin described above may also be one in which at least some of the hydrogen atoms in the constituent unit thereof have been replaced with a substituent having an electric charge or a substituent capable of forming a covalent bond. Such thermosetting resins may be synthesized by using as a raw material a monomer in which at least one hydrogen atom has been replaced (derivatized) by the substituent group described above by a known method.

Such thermosetting resins may be synthesized according to known methods. For example, a melamine formaldehyde resin may be synthesized by polycondensing methylol melamine prepared in advance as described above by heating after adding a reaction accelerator such as an acid as needed.

### (Thermoplastic Resin)

The binder according to the present invention may be a thermoplastic resin. The thermoplastic resin includes, for example, a monofunctional monomer (a monomer having one group involved in a polymerization reaction in one molecule, in the above example, a vinyl group) of at least one type selected from the group consisting of (meth)acrylic acid and its alkyl esters, acrylonitrile, and derivatives thereof. Any one of these monomers may be used alone, or a combination of two or more monomers may be used.

If desired, one or more comonomers other than the above compounds may be further used in combination. The thermoplastic resin described above may contain a constituent unit formed from a multifunctional monomer such as divinylbenzene (a monomer having two or more groups involved in a polymerization reaction in one molecule, in the above example, two or more vinyl groups), for example, a cross-linking moiety. For example, a crosslinked product of polymethyl methacrylate may be cited.

Furthermore, the above thermoplastic resin may contain a structural unit having a functional group for surface modification of the luminescent nanoparticles of the present invention. For example, by using a monomer such as glycidyl methacrylate having an epoxy group as a raw material, luminescent nanoparticles with an epoxy group oriented on the surface may be prepared. The epoxy group may be converted to an amino group by reacting with excess ammonia water. Various biomolecules may be introduced into the amino group formed in this way according to known methods (via a linker molecule, when needed).

### <1.6> Method for Producing Luminescent Nanoparticles

The luminescent nanoparticles of the present invention may be produced according to the polymerization process known for various binders, after using a luminescent dye that meets specific conditions.

### (Polymerization Process)

The polymerization process is a process in which a reaction mixture containing a luminescent dye, a resin raw material (monomer, oligomer or prepolymer), preferably also a surfactant and a polymerization reaction accelerator is heated to progress a polymerization reaction of the resin to generate resin particles containing the luminescent dye.

The order of addition of each component in the reaction mixture is not particularly limited. Typically, the surfactant is added to the aqueous solution of the luminescent dye, followed by adding the resin raw material, and finally the polymerization reaction accelerator is added. Alternatively, the sequence may be as follows. The resin raw material is added to the aqueous solution of the surfactant, followed by adding the polymerization reaction accelerator to proceed the synthesis reaction of the resin particles, and then the aqueous solution of the luminescent dye is added. The concentration of the aqueous solution of the specific luminescent dye according to the present invention used in such a polymerization process may be adjusted in a relatively higher range (e.g., 2,500 to 10,000 μM) than that of conventional aqueous solutions of luminescent dyes.

The conditions (temperature and time) of the polymerization reaction may be set appropriately, taking into consideration the type of resin and composition of the raw material mixture.

As for the polymerization method, as long as it is a known polymerization method, it is not particularly limited. The known polymerization methods include, for example, a bulk polymerization, an emulsion polymerization, a soap-free emulsion polymerization, a seed polymerization, and a suspension polymerization. In the case of the bulk polymerization, resin particles of a desired particle size may be obtained by classifying after pulverization. Emulsion polymerization is a polymerization method in which a medium such as water, a monomer that is difficult to dissolve in the medium, and an emulsifier (surfactant) are mixed, and then a polymerization initiator that is soluble in the medium is added. It is characterized by a small variation in the particle size obtained.

The soap-free emulsion polymerization is an emulsion polymerization in which no emulsifier is used. It has a feature that particles of uniform diameter may be obtained. The seed polymerization is a polymerization in which seed particles made separately are added at the start of polymerization. It is characterized in that the particle size, particle size distribution, and quantity (number) as seed particles are arbitrarily set for polymerization, and polymerization may be performed aiming at the desired particle size and particle size distribution. The suspension polymerization is a polymerization method in which the monomer and water as a solvent are mechanically stirred and suspended. It is characterized in that it is possible to obtain particles with a small size and a uniform shape.

As a specific example, taking the synthesis of thermosetting resins such as a melamine resin, the reaction temperature is usually 70 to 200° C. and the reaction time is usually 2 to 120 minutes. It is appropriate to set the reaction temperature to a temperature at which the performance of the luminescent dye is not degraded (within the heat-resistant temperature range). The heating may be carried out in multiple stages, for example, the reaction may be carried out at a relatively low temperature for a certain period of time, followed by a temperature increase and a reaction is carried out at a relatively low temperature for a certain period of time.

After the polymerization reaction is completed, impurities such as excess resin material, luminescent dye, and surfactant are removed from the reaction solution, and the generated luminescent nanoparticles are collected and purified. For example, the reaction solution is centrifuged to remove the supernatant containing impurities, and then ultrapure water is added and ultrasonically irradiated to disperse and wash it again. These operations should be repeated multiple times until no light absorption or emission derived from the resin or luminescent dye is observed in the supernatant.

The luminescent nanoparticles using the thermosetting resin may basically be produced according to the emulsion polymerization method, but it is preferred to produce them by the polymerization process as described above using a surfactant and a polymerization reaction accelerator. In the luminescent nanoparticles obtained by such a production method, most, preferably substantially all of the luminescent dye is immobilized in a state in which the luminescent dye is contained in the resin particles, but it is not excluded that some of the luminescent dye is immobilized in a state bound or attached to the surface of the resin particles.

There is no limitation as to what kind of chemical or physical action is used to immobilize the luminescent dye to the resin particle in the state in which the luminescent dye is contained. In the present invention, prior to the polymerization process, it is not necessary to provide a derivatization process to covalently bond the resin material to the luminescent dye in advance or to introduce an actively charged substituent into the resin material (even without such a process, luminescent nanoparticles with excellent luminescence intensity and lightfastness are obtained), but it is not excluded to use such steps together if desired.

### (Surfactant)

As a surfactant, known emulsifiers for emulsion polymerization may be used. Examples of the surfactant include anionic (anion-based), nonionic (non-ionic), and cationic (cation-based) surfactants. When synthesizing a thermosetting resin having a positively charged substituent or moiety (cationic), it is preferable to use an anionic or nonionic surfactant. Conversely, when synthesizing a (anionic) thermosetting resin having a negatively charged substituent or moiety, it is preferable to use a cationic or nonionic surfactant.

Anionic surfactants include, for example, sodium dodecylbenzenesulfonate (product name “NEOPELEX” series, Kao Corporation). Nonionic surfactants include, for example, polyoxyethylene alkyl ether-based compounds (product name: “EMULGEN series”, Kao Corporation), polyvinyl pyrrolidone (PVP), and polyvinyl alcohol (PVA). Cationic surfactants include, for example, dodecyl trimethyl ammonium bromide.

By adjusting the amount of surfactant added, the particle size of the resin particles may be adjusted and the coefficient of variation of the particle size may be small, that is, luminescent nanoparticles with uniform particle size may be produced. The amount of surfactant added is, for example, 10 to 60 mass % of the resin raw materials, or 0.1 to 3.0 mass % of the total raw material mixture. Increasing the amount of surfactant added tends to reduce the particle size, while decreasing the amount of surfactant added tends to increase the particle size.

### (Polymerization Reaction Accelerator)

A polymerization reaction accelerator has a function of accelerating the polycondensation reaction of the thermosetting resin such as a melamine resin, as well as giving a proton (H⁺) to the functional group such as an amino group contained in the resin or luminescent dye and charging it to facilitate electrostatic interaction. Although the reaction of the thermosetting resin proceeds only by heating, but it proceeds at a lower temperature when a polymerization reaction accelerator is added, so that the polymerization reaction accelerator may be added to the extent that the reaction and performance may be controlled. Such polymerization reaction accelerators include, for example, acids such as formic acid, acetic acid, sulfuric acid, paratoluenesulfonic acid, and dodecylbenzenesulfonic acid. When the luminescent dye is a compound having a carboxy group or a sulfo group, the luminescent dye may also donate a proton as well as the above acids.

### 2. Luminescent Labeling Material for Pathological Diagnosis

The luminescent labeling material for pathological diagnosis of the present invention is characterized in that a target-directed ligand is covalently bonded to the surface of the luminescent nanoparticle of the present invention as described above.

Although the uses of the luminescent nanoparticles of the present invention are not particularly limited, they are typically used as a luminescent labeling material for pathological diagnosis to label a substance to be detected in a sample (tissue section) and to enable fluorescent observation in immunostaining. In other words, the luminescent nanoparticles of the present invention as described above are suitable to be used as a complex (conjugate) by linking a target-directed ligand according to the embodiment of immunostaining.

Although the substances to be detected are not particularly limited, in pathological diagnosis, antigens are generally selected according to the purpose. For example, in the pathological diagnosis of breast cancer, HER2 may be used as the target substance for detection. The target substance for detection does not have to be specific to the organism. For example, the target substance for detection may be a drug.

### <2.1> Target-Directed Ligand

In the present invention, a “target-directed ligand” is a molecule having specific binding properties to a specific tissue or cell (substance to be detected). It is preferred that the target-directed ligand of the present invention is a molecule selected from the group consisting of an antibody, a cell organelle affinity substance, and a protein having a binding property with a sugar chain, in terms of suppressing nonspecific adsorption.

The type of target-directed ligand is not particularly limited, and the most suitable one may be selected according to the purpose. The target-directed ligands specifically include the following.

A first example of a target-directed ligand is a primary antibody (an antibody that specifically binds to a substance to be detected). A luminescent labeling material for pathological diagnosis in which the target-directed ligand is a primary antibody may be fluorescently labeled by directly binding to the substance to be detected (primary antibody method).

A second example of a target-directed ligand is a secondary antibody (an antibody that binds to a primary antibody). For example, if the primary antibody is an antibody (IgG) produced from a rabbit, the secondary antibody is an anti-rabbit IgG antibody. The primary antibody that binds to the substance to be detected may be indirectly fluorescently labeled with the substance to be detected by the binding of a luminescent labeling material for pathological diagnosis whose target-directed ligand is the secondary antibody (secondary antibody method).

A third example of a target-directed ligand is avidin, streptavidin or biotin. For example, when a luminescent labeling material for pathological diagnosis in which avidin or streptavidin is used for a target-directed ligand, a secondary antibody-biotin complex is used in combination. A secondary antibody-biotin complex binds to the primary antibody that is bound to the substance to be detected, and the complex is further bound to a luminescent labeling material for pathological diagnosis in which avidin or streptavidin is a targeting ligand. Thus, the substance to be detected may be indirectly fluorescently labeled (biotin-avidin method or sandwich method). Conversely, a pathological diagnostic luminescent labeling material in which the target-directed ligand is biotin may be combined with a secondary antibody-avidin complex or a secondary antibody-streptavidin.

The primary antibody that specifically binds to the selected substance to be detected should be selected. For example, when the substance to be detected is HER2, an anti-HER2 monoclonal antibody may be used as a primary antibody. Such a primary antibody (monoclonal antibody) may be produced by a general method using mouse, rabbit, cow, goat, sheep, dog, and chicken as immunizing animals.

The secondary antibody that binds to the selected primary antibody should be selected. For example, when the primary antibody is a rabbit anti-HER2 monoclonal antibody, an anti-rabbit IgG antibody may be used as a secondary antibody. Such secondary antibodies may also be produced by general methods.

In addition, it is also possible to use a nucleic acid molecule as the substance to be detected and a nucleic acid molecule having a base sequence complementary to the nucleic acid molecule as the corresponding target-directed ligand.

The luminescent labeling material for pathological diagnosis may be prepared by any known method. For example, amidation by reaction of an amine with a carboxylic acid, sulfidation by reaction of a maleimide with a thiol, imination by reaction of an aldehyde with an amine, and amination by reaction of an epoxy with an amine may be used. The functional groups involved in such reactions may be those pre-existing on the surface of the luminescent nanoparticles (functional groups derived from the raw monomer of the binder) or may be functional groups converted from the functional groups existing on the surface of the luminescent nanoparticles according to known methods or introduced by surface modification. If necessary, an appropriate linker molecule may be utilized.

In another aspect of the invention, a kit for tissue immunostaining using the luminescent nanoparticles of the invention is provided. The kit contains at least the luminescent labeling material for pathological diagnosis of the present invention or the luminescent nanoparticles of the present invention, a target-directed ligand, and reagents. The kit may further contain primary antibodies, secondary antibodies, other target-directed ligands (e.g., biotin) used in combination with the target-directed ligands (e.g., streptavidin), reagents for forming the desired complex, and other reagents used in immunohistochemical staining.

### <2.2> Method for Producing Luminescent Labeling Material for Pathological Diagnosis

In the technical field to which the present invention belongs, various methods are known for producing a luminescent labeling material for pathological diagnosis by combining a luminescent labeling material (luminescent nanoparticle in the present invention) with a target-directed ligand via a covalent bond, and such a method may be used in the present invention.

For example, reactions between reactive functional groups such as a carboxy group, an amino group, an aldehyde group, a thiol group, and a maleimide groups may be used to covalently link a luminescent labeling material for pathological diagnosis (one reactive functional group present on its surface) and a target-directed ligand (the other reactive functional group present in its molecule) via a covalent bond. If the functional groups they possess cannot be directly bonded to each other, they may also be bonded via a “linker molecule” having a predetermined functional group at each end of the molecule. Such a reaction may be carried out by adding the necessary reagents and allowing a predetermined time to elapse.

Specific example is as follows. Luminescent nanoparticles having a hydroxy group on the surface are reacted with a silane coupling agent (e.g., aminopropyltrimethoxysilane) to introduce an amino group, while streptavidin is reacted with a thiol group-introducing reagent (e.g., N-succinimidyl S-methylthioacetate) to introduce a thiol group. Finally, a PEG (polyethylene glycol)-based linker molecule having maleimide groups at both ends that are reactive with both an amino group and a thiol group is reacted to link the luminescent nanoparticles and streptavidin.

In addition, when a resin (acrylic resin) is synthesized using glycidyl methacrylate as a raw material monomer, an epoxy group derived from the monomer appears on the surface of the luminescent nanoparticles. By adding ammonia water to these luminescent nanoparticles, the epoxy group may be converted to an amino group, and the desired target-directed ligands may be linked to the amino group.

### EXAMPLES

Hereinafter, the present invention will be specifically described with examples, but the present invention is not limited thereto. In the examples, “part” or “%” is used. It indicates “part by mass” or “mass %” unless otherwise specified.

### <<Luminescent Dye>>

### <Luminescent Dyes Used in Comparative Example>

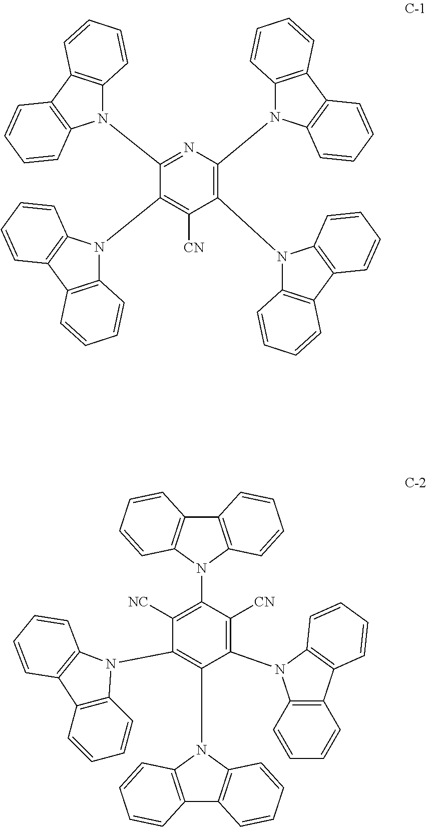 [SMILES: N#Cc1c(-n2c3ccccc3c3ccccc32)c(-n2c3ccccc3c3ccccc32)nc(-n2c3ccccc3c3ccccc32)c1-n1c2ccccc2c2ccccc21 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

### <Luminescent Dyes Used in Example 1>

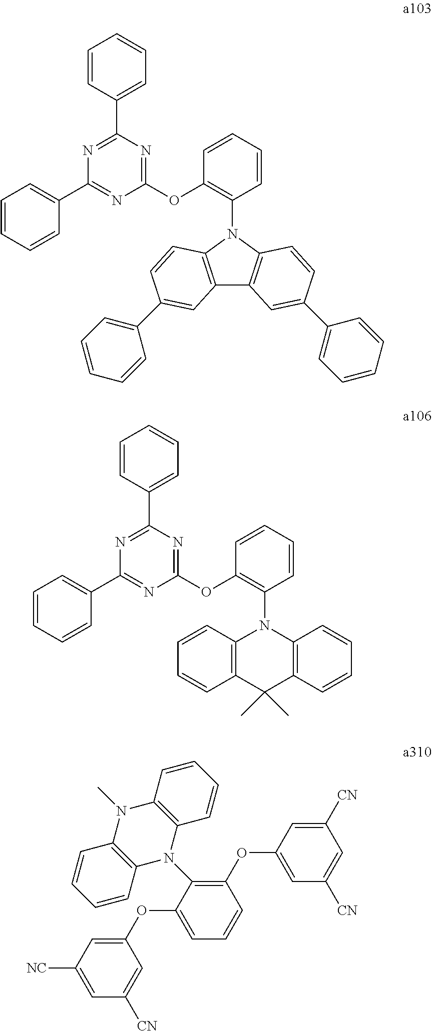 [SMILES: c1ccc(-c2ccc3c(c2)c2cc(-c4ccccc4)ccc2n3-c2ccccc2Oc2nc(-c3ccccc3)nc(-c3ccccc3)n2)cc1 ; CC1(C)c2ccccc2N(c2ccccc2Oc2nc(-c3ccccc3)nc(-c3ccccc3)n2)c2ccccc21 ; CN1c2ccccc2N(c2c(Oc3cc(C#N)cc(C#N)c3)cccc2Oc2cc(C#N)cc(C#N)c2)c2ccccc21]

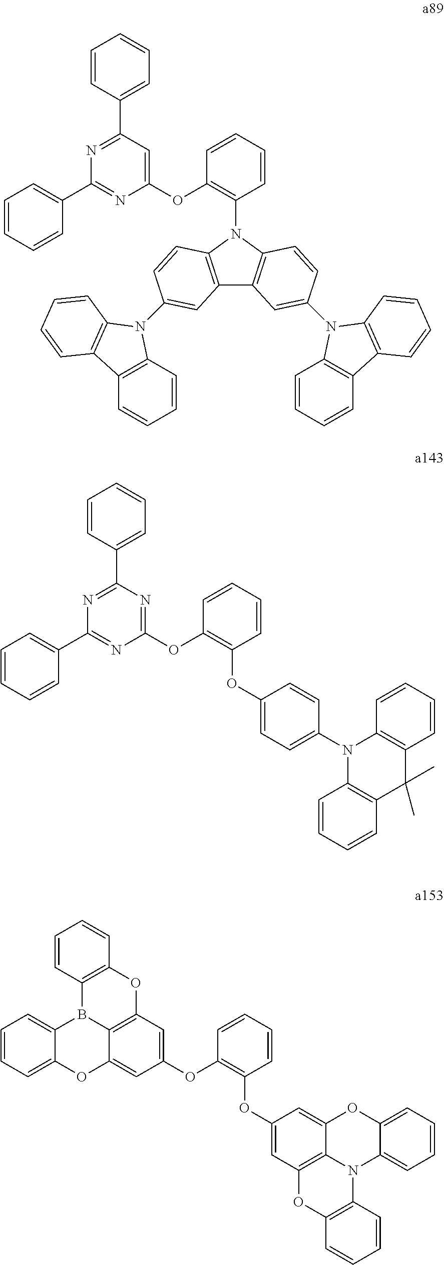 [SMILES: c1ccc(-c2cc(Oc3ccccc3-n3c4ccc(-n5c6ccccc6c6ccccc65)cc4c4cc(-n5c6ccccc6c6ccccc65)ccc43)nc(-c3ccccc3)n2)cc1 ; CC1(C)c2ccccc2N(c2ccc(Oc3ccccc3Oc3nc(-c4ccccc4)nc(-c4ccccc4)n3)cc2)c2ccccc21 ; c1ccc(Oc2cc3c4c(c2)Oc2ccccc2N4c2ccccc2O3)c(Oc2cc3c4c(c2)Oc2ccccc2B4c2ccccc2O3)c1]

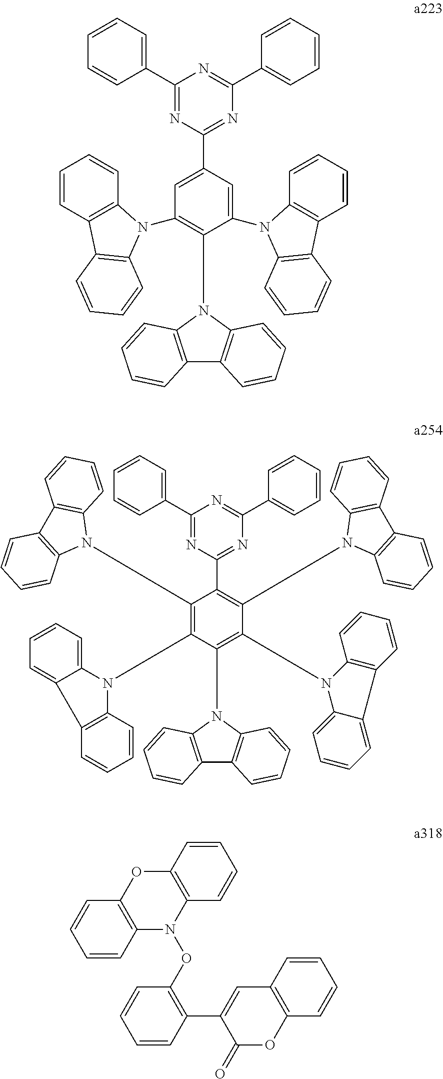 [SMILES: c1ccc(-c2nc(-c3ccccc3)nc(-c3cc(-n4c5ccccc5c5ccccc54)c(-n4c5ccccc5c5ccccc54)c(-n4c5ccccc5c5ccccc54)c3)n2)cc1 ; c1ccc(-c2nc(-c3ccccc3)nc(-c3c(-n4c5ccccc5c5ccccc54)c(-n4c5ccccc5c5ccccc54)c(-n4c5ccccc5c5ccccc54)c(-n4c5ccccc5c5ccccc54)c3-n3c4ccccc4c4ccccc43)n2)cc1 ; O=c1oc2ccccc2cc1-c1ccccc1ON1c2ccccc2Oc2ccccc21]

### <Luminescent Dyes Used in Example 2>

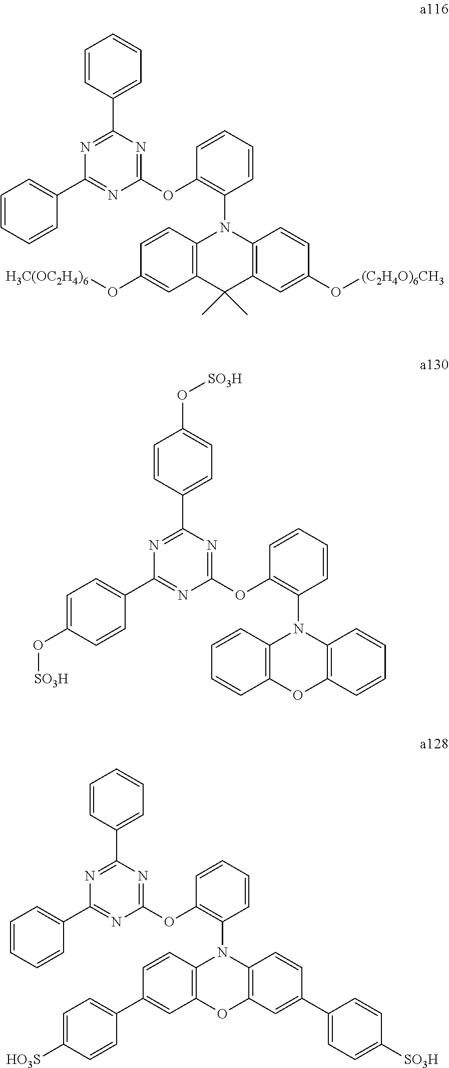

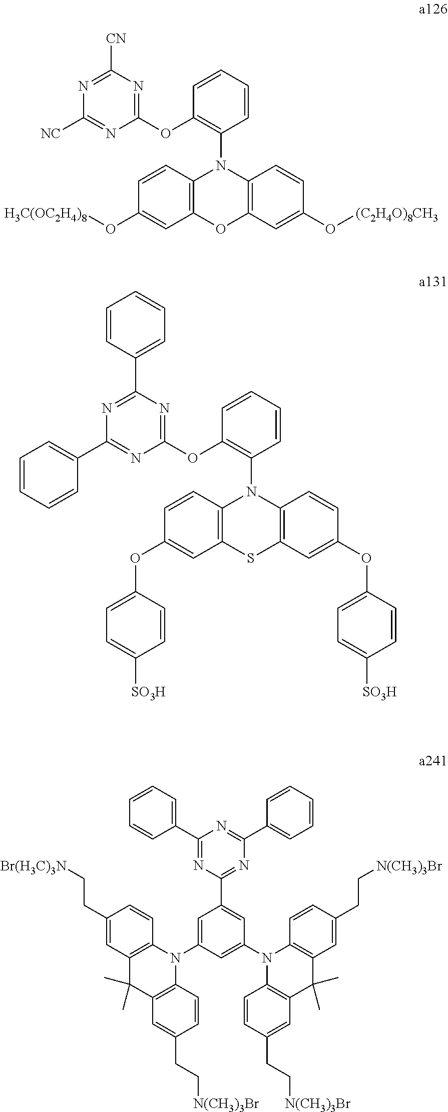

### <Luminescent Dyes Used in Example 3>

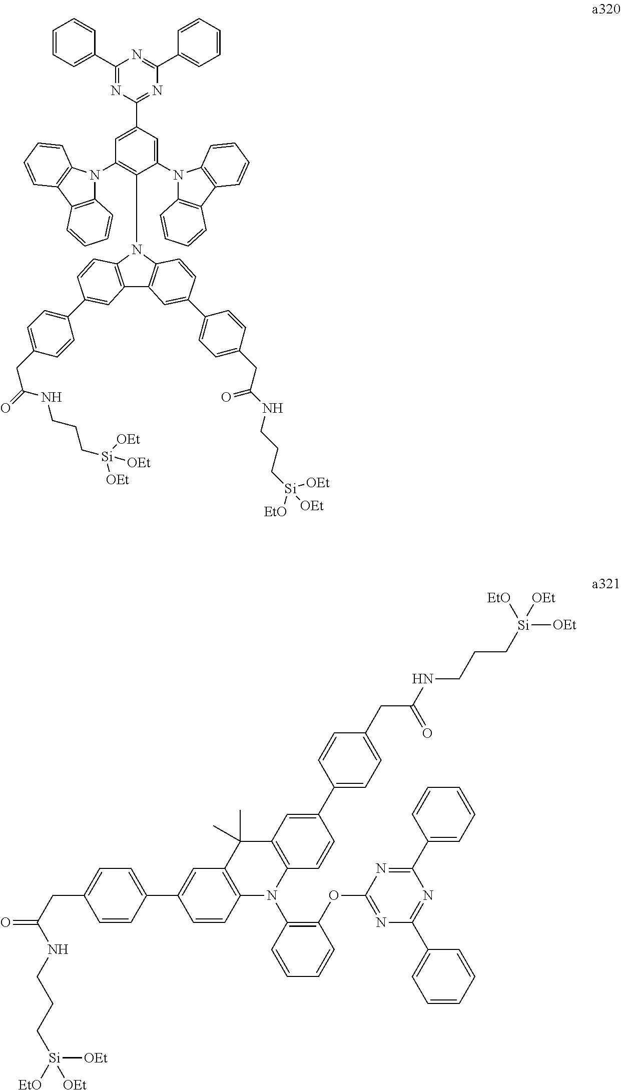 [SMILES: CCO[Si](CCCNC(=O)Cc1ccc(-c2ccc3c(c2)c2cc(-c4ccc(CC(=O)NCCC[Si](OCC)(OCC)OCC)cc4)ccc2n3-c2c(-n3c4ccccc4c4ccccc43)cc(-c3nc(-c4ccccc4)nc(-c4ccccc4)n3)cc2-n2c3ccccc3c3ccccc32)cc1)(OCC)OCC ; CCO[Si](CCCNC(=O)Cc1ccc(-c2ccc3c(c2)C(C)(C)c2cc(-c4ccc(CC(=O)NCCC[Si](OCC)(OCC)OCC)cc4)ccc2N3c2ccccc2Oc2nc(-c3ccccc3)nc(-c3ccccc3)n2)cc1)(OCC)OCC]

### <Luminescent Dye Used in Example 4>

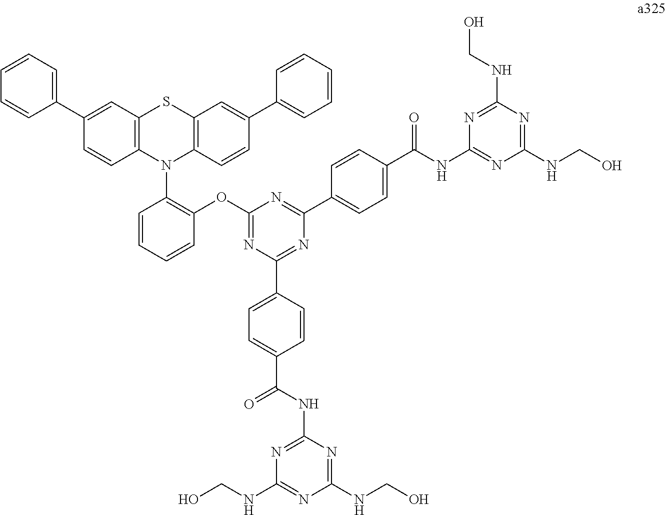 [SMILES: O=C(Nc1nc(NCO)nc(NCO)n1)c1ccc(-c2nc(Oc3ccccc3N3c4ccc(-c5ccccc5)cc4Sc4cc(-c5ccccc5)ccc43)nc(-c3ccc(C(=O)Nc4nc(NCO)nc(NCO)n4)cc3)n2)cc1]

### <<Preparation of Luminescent Nanoparticles>>

### [Example 1 and Comparative Example] Preparation of Luminescent Nanoparticles Using Oil-Soluble Dyes

### <Preparation of Luminescent Nanoparticles Nos. (1-1) to (1-22)>

To a solution of a luminescent dye (types and amounts added as listed in Table I) dissolved in 0.2 mL of dichloromethane, a surfactant (EMULGEN 430) was added to become 0.5 vol % by adding 20 mL of water. The solution was stirred on a hot stirrer and the temperature was raised to 40° C., after which 1.2 g of melamine resin was added.

The luminescent nanoparticles were prepared by adding 0.02 mmol of dodecylbenzenesulfonic acid to the solution. The resulting dispersion was centrifuged (20000 G for 90 minutes) to collect the particles and purified by washing with ultrapure water. The process was repeated five times by removing the supernatant after centrifugation and redispersing in ultrapure water to obtain luminescent nanoparticles Nos. (1-1) to (1-22) with an average particle size of 130 nm.

### [Example 2] Preparation of Luminescent Nanoparticles Using Water-Soluble Dyes

### <Preparation of Luminescent Nanoparticles Nos. (2-1) to (2-12)>

To a solution of a luminescent dye (types and amounts added as listed in Table I) dissolved in 0.2 mL of water, a surfactant (EMULGEN 409PV) was added to become 0.5 vol % by adding 20 mL of water. The solution was stirred on a hot stirrer and the temperature was raised to 80° C., and then 2.0 g of melamine resin was added. Except the change of the temperature and the amount of melamine resin, the same operation as in Example 1 was carried out to produce luminescent nanoparticles Nos. (2-1) to (2-12) with an average particle size of 130 nm.

### [Example 3] Preparation of Luminescent Nanoparticles Having a Covalent Bond to a Binder

### <Preparation of Luminescent Nanoparticles Nos. (3-1) to (3-4)>

The luminescent dye (types and amounts added as listed in Table I) was mixed with 48 mL of 99% ethanol, 0.6 mL of tetraethoxysilane (TEOS), 2 mL of ultrapure water, and 2.0 mL of 28 mass % ammonia water at 5° C. for 3 hours.

The mixture prepared in the above process was centrifuged at 10000 G for 20 minutes and the supernatant was removed. Ethanol was added to the precipitate to disperse the precipitate, and a rinse operation was performed to centrifuge the precipitate again. The same rinsing was repeated twice, and luminescent nanoparticles Nos. (3-1) to (3-4) (silica particles containing a luminescent dye) with an average particle size of 130 nm were prepared.

### [Example 4] Preparation of Luminescent Nanoparticles Having a Covalent Bond to a Binder

### <Preparation of Luminescent Nanoparticles Nos. (4-1) to (4-2)>

The luminescent nanoparticles No. (4-1) to (4-2) to (4-3) with an average particle size of 130 nm were prepared under the same conditions as in Example 1, except that the luminescent dye (types and amounts added shown in Table I) was changed.

### <<Evaluation of Luminescent Nanoparticles>>

The luminescent nanoparticles with oil-soluble dyes prepared in Example 1, the luminescent nanoparticles with water-soluble dyes prepared in Example 2, and the luminescent nanoparticles a covalent bond with a binder prepared in Examples 3 and 4 were evaluated. In the evaluation of the luminescent nanoparticles, the quantum yield and luminance were measured, and their relative values were summarized in Table I. The relative values for each particle are values by setting the measured value of the luminescent nanoparticle No. 1 (1-1) to 100.

The quantum yields were measured as follows. Luminescent nanoparticles were dispersed in PBS so that the particle molar concentration was 0.01 mmol/L. The quantum yield of the dispersion was measured by excitation at the maximum absorption wavelength of each nanoparticle using an absolute PL quantum yield measurement device C9920-02 (Hamamatsu Photonics, Inc.).

The luminance was measured as follows. Luminescent nanoparticles were dispersed in PBS so that the particle molar concentration was 0.01 mmol/L. The luminance of the dispersion was measured with a fluorescence spectrophotometer (F-7000; Hitachi High-Technologies Corporation) at room temperature by excitation at the maximum absorption wavelength of each nanoparticle.

Since the luminescent nanoparticles of the present invention have solid-state luminescence, the quantum yield increased or was maintained when the amount of dye added was increased. Furthermore, since the absorbance also increased as the amount of dye added increased, the luminance increased in both cases. As a result, the luminescent nanoparticles of the present invention were superior to the luminescent nanoparticles of the comparative examples, and the effectiveness of the present invention was confirmed.

In the comparative example of luminescent nanoparticle No. (1-2), since the luminescent dye (C-1) has an aggregation-induced luminescence property, the quantum yield and luminance of the luminescent nanoparticles were increased with increasing the amount of dye added compared to No. (1-1), but the increase was limited in its range.

In the comparative example of luminescent nanoparticle No. (1-4), both quantum yield and luminance were decreased with increasing the amount of dye added compared to No. (1-3) due to concentration quenching when the amount of dye added was increased.

TABLE I

| Luminescent | | Added amount | | | |
|---|---|---|---|---|---|
| nanoparticles | | of dye | Relative | Relative | |
| No. | Dye | (μmol) | quantum yield | luminance | Remarks |
| 1-1 | C-1 | 50 | 100 | 100 | Comparative Example |
| 1-2 | C-1 | 200 | 155 | 210 | Comparative Example |
| 1-3 | C-2 | 50 | 195 | 261 | Comparative Example |
| 1-4 | C-2 | 200 | 71 | 141 | Comparative Example |
| 1-5 | a103 | 50 | 223 | 301 | Present Invention |
| 1-6 | a103 | 200 | 251 | 461 | Present Invention |
| 1-7 | a106 | 50 | 270 | 360 | Present Invention |
| 1-8 | a106 | 200 | 281 | 642 | Present Invention |
| 1-9 | a310 | 50 | 265 | 377 | Present Invention |
| 1-10 | a310 | 200 | 291 | 599 | Present Invention |
| 1-11 | a89 | 50 | 309 | 409 | Present Invention |
| 1-12 | a89 | 200 | 314 | 608 | Present Invention |
| 1-13 | a143 | 50 | 299 | 400 | Present Invention |
| 1-14 | a143 | 200 | 305 | 578 | Present Invention |
| 1-15 | a153 | 50 | 345 | 421 | Present Invention |
| 1-16 | a153 | 200 | 368 | 601 | Present Invention |
| 1-17 | a223 | 50 | 327 | 406 | Present Invention |
| 1-18 | a223 | 200 | 340 | 587 | Present Invention |
| 1-19 | a254 | 50 | 401 | 467 | Present Invention |
| 1-20 | a254 | 200 | 420 | 621 | Present Invention |
| 1-21 | a318 | 50 | 290 | 381 | Present Invention |
| 1-22 | a318 | 200 | 412 | 523 | Present Invention |
| 2-1 | a116 | 50 | 301 | 410 | Present Invention |
| 2-2 | a116 | 200 | 301 | 608 | Present Invention |
| 2-3 | a130 | 50 | 251 | 374 | Present Invention |
| 2-4 | a130 | 200 | 260 | 624 | Present Invention |
| 2-5 | a128 | 50 | 245 | 437 | Present Invention |
| 2-6 | a128 | 200 | 295 | 641 | Present Invention |
| 2-7 | a126 | 50 | 223 | 360 | Present Invention |
| 2-8 | a126 | 200 | 270 | 634 | Present Invention |
| 2-9 | a131 | 50 | 260 | 403 | Present Invention |
| 2-10 | a131 | 200 | 265 | 678 | Present Invention |
| 2-11 | a241 | 50 | 241 | 398 | Present Invention |
| 2-12 | a241 | 200 | 241 | 630 | Present Invention |
| 3-1 | a320 | 50 | 294 | 347 | Present Inventior |
| 3-2 | a320 | 200 | 315 | 460 | Present Invention |
| 3-3 | a321 | 50 | 308 | 388 | Present Invention |
| 3-4 | a321 | 200 | 343 | 500 | Present Invention |
| 4-1 | a325 | 50 | 320 | 364 | Present Invention |
| 4-2 | a325 | 200 | 350 | 603 | Present Invention |

The luminescent nanoparticles of the examples were dispersed in water, and absorption and emission spectra measurements and delayed luminescence measurements were carried out. The results are shown in Table II. In Table II, for long Stokes shift luminescence, the Stokes shift of 100 to 200 nm is indicated as “Circle”, and the Stokes shift of 200 nm or more is indicated as “Double circle”. The delayed luminescence properties were observed for all the luminescent nanoparticles in Table II. When the ratio of delayed to immediate luminescence intensity after 100 ns is 1/10 or more, it is indicated as “Double circle”, and when it is less than 1/10, it is indicated as “Circle”.

The delayed luminescence measurements were performed using a streak camera C4334 (Hamamatsu Photonics, Inc.) while the dispersion was excited by laser light.

TABLE II

| Luminescent | | Addedamount | Long Stokes | Luminance intensity | |
|---|---|---|---|---|---|
| nanoparticles | | of dye | shift | ratio of | |
| No. | Dye | (μmol) | luminescence | (Delayed/Immediate) | Remarks |
| 1-5 | a103 | 50 | ⊚ | ⊚ | Present Invention |
| 1-7 | a106 | 50 | ⊚ | ⊚ | Present Invention |
| 1-9 | a310 | 50 | ⊚ | ⊚ | Present Invention |
| 1-12 | a89 | 200 | ⊚ | ⊚ | Present Invention |
| 1-14 | a143 | 200 | ⊚ | ⊚ | Present Invention |
| 1-16 | a153 | 200 | ⊚ | ⊚ | Present Invention |
| 1-17 | a223 | 50 | ◯ | ◯ | Present Invention |
| 1-19 | a254 | 50 | ◯ | ◯ | Present Invention |
| 1-21 | a318 | 50 | ⊚ | ⊚ | Present Invention |
| 2-1 | a116 | 50 | ⊚ | ⊚ | Present Invention |
| 2-3 | a130 | 50 | ⊚ | ⊚ | Present Invention |
| 2-5 | a128 | 50 | ⊚ | ⊚ | Present Invention |
| 2-7 | a126 | 50 | ⊚ | ⊚ | Present Invention |
| 2-10 | a131 | 200 | ⊚ | ⊚ | Present Invention |
| 2-12 | a241 | 200 | ◯ | ◯ | Present Invention |
| 3-1 | a320 | 50 | ◯ | ◯ | Present Invention |
| 3-4 | a321 | 200 | ⊚ | ⊚ | Present Invention |
| 4-1 | a325 | 50 | ⊚ | ⊚ | Present Invention |

As shown in Table II, the long Stokes shift luminescence and delayed luminescence properties were confirmed in all the example particles. Among them, the long Stokes shift was more than 200 nm in the intramolecular exciplex dye-containing particles. Furthermore, the ratio of delayed to immediate luminescence intensity was higher for the intramolecular exciplex dye-containing particles than for the heat-active delayed fluorescent dye-containing particles.

### <<Preparation of Luminescent Labeling Materials for Pathological Diagnosis>>

### [Example 5] Luminescent Labeling Materials for Pathological Diagnosis Composed of Luminescent Nanoparticles Nos. (1-1) to (1-22), (2-1) to (2-12), and (4-1) to (4-2)

<Preparation of Luminescent Nanoparticles Surface-Modified with a PEG Chain Having a Maleimide Group at the End>

The above luminescent nanoparticles Nos. (1-1) to (1-22), (2-1) to (2-12), and (4-1) to (4-2), which are melamine particles containing luminescent dyes, each were taken in an amount of 0.1 mg, and dispersed in 1.5 mL of ethanol. Then, they were mixed with 2 μL of aminopropyltrimethoxysilane “LS-3150” (Shin-Etsu Chemical Co., Ltd.) and the reaction was carried out at room temperature with stirring for 8 hours to perform surface amination treatment. The concentration of luminescent nanoparticles with aminated surface was adjusted to 3 nM using PBS (phosphate buffered physiological saline) containing 2 mM of EDTA (ethylenediaminetetraacetic acid). To this solution was added the linker reagent “SM(PEG)12” (Thermo Scientific, Inc. Cat. No. 22112) and mixed to a final concentration of 10 mM, and the reaction was carried out at room temperature for 1 hour with stirring.

The reaction solution was centrifuged at 10,000 G for 20 minutes to remove the supernatant, and then PBS containing 2 mM of EDTA was added to disperse the sediment and centrifuged again under the same conditions. The luminescent nanoparticles surface-modified with a PEG chain having a maleimide group at the end were obtained by washing three times using the same procedure.

<Preparation of Streptavidin Introduced with a Thiol Group>

First, to 40 μL of an aqueous solution of streptavidin (Wako Pure Chemical Industries, Ltd.) adjusted to 1 mg/mL was added 70 μL of an aqueous solution of N-succinimidyl-S-acetyl thioacetate (SATA, Pirce Corporation) adjusted to 64 mg/mL. The reaction was allowed to proceed at room temperature for 1 hour to convert the amino group of the streptavidin to the protected thiol group (—NH—CO—CH₂—S—CO—CH₃).

Subsequently, hydroxylamine treatment was used to generate a free thiol group (—SH) from the protected thiol group to complete the process of introducing a thiol group (—SH) to streptavidin. The solution was then passed through a gel filtration column (Zaba Spin Desalting Columns: Funakoshi) to desalinate the solution to obtain a thiol group-introduced streptavidin.

<Preparation of Luminescent Nanoparticles Modified with Streptavidin>

The prepared luminescent nanoparticles surface-modified with a PEG chain having a maleimide groups at the end and the prepared streptavidin introduced with a thiol group were mixed in PBS containing 2 mM of EDTA and allowing them to react for 1 hour. Thus, streptavidin was bonded via a PEG chain to the luminescent nanoparticles. The reaction was stopped by adding 10 mM of mercaptoethanol to the reaction solution. The resulting solution was concentrated by centrifugal filtering, and unreacted material was removed using a purifying gel filtration column to obtain a luminescent labeling material (streptavidin-modified luminescent nanoparticles) for pathological diagnosis.

### [Example 6] Luminescent Labeling Materials for Pathological Diagnosis Composed of Luminescent Nanoparticles Nos. (3-1) to (3-4)

<Preparation of Luminescent Nanoparticles with a Maleimide Group at the End>

For each of the above luminescent nanoparticles Nos. (3-1) to (3-4), which are silica particles containing luminescent dyes, the solution was adjusted to 3 nM using PBS containing 2 mM of EDTA (ethylenediaminetetraacetic acid). SM(PEG) 12 (manufactured by Thermo Scientific, succinimidyl-[(N-maleimidopropionamide)-dodecane ethylene glycol] ester) was mixed with this solution to make a final concentration of 10 mM and reacted at 5° C. for 1 hour.

This mixed solution was centrifuged at 10,000 G for 20 minutes, and after removing the supernatant, PBS containing 2 mM of EDTA was added to disperse the sediment, followed by centrifugation again. By performing washing by the same procedure three times, luminescent nanoparticles with a maleimide group at the end were obtained.

<Preparation of Streptavidin Introduced with a Thiol Group>

Streptavidin capable of binding to luminescent nanoparticles was prepared as follows.

First, 40 μL of streptavidin (Wako Pure Chemical Industries, Ltd.) adjusted to 1 mg/mL was L was added to 210 μL of borate buffer. Then, 70 μL of 2-iminothiolane hydrochloride (Sigma-Aldrich Co., Ltd.), adjusted to 64 mg/mL, was added. The mixture was allowed to react at room temperature for 1 hour. By this, the thiol group (—NH—C(═NH₂⁺Cl⁻)—CH₂—CH₂—CH₂—SH) was introduced to the amino group of streptavidin.

This streptavidin solution was then transferred to a gel filtration column (Zaba Spin Desalting Columns: Funakoshi) to obtain streptavidin that is capable of binding to the above silica particles.

<Preparation of Luminescent Nanoparticles Modified with Streptavidin>

The total amount of streptavidin (containing 0.04 mg) and 740 μL of the luminescent nanoparticles (silica particles) adjusted to 0.67 nM using PBS containing 2 mM of EDTA were mixed and reacted at room temperature for 1 hour. Further, 10 mM of mercaptoethanol was added to stop the reaction. The resulting solution was concentrated by centrifugal filtering, and unreacted streptavidin and other substances were removed using a purifying gel filtration column to obtain a luminescent labeling material for pathological diagnosis (streptavidin-modified luminescent nanoparticles (silica particles containing luminescent dye)).

### Example 7: Evaluation of Luminescent Labeling Materials for Pathological Diagnosis

### <Tissue Staining Process>

### [Immunohistochemical Staining]

Immunostaining of human breast tissue was performed using a staining agent for tissue staining containing the luminescent labeling material for pathological diagnosis composed of the luminescent nanoparticles produced in Examples 5 and 6. Here, the staining agent for tissue staining was prepared using a PBS buffer containing 1% BSA. A tissue array slide (manufactured by Cosmo Bio, product number CB-A712) was used for the stained section. For the stained sections, the FISH score for each spot was calculated in advance using Pathvysion HER2 DNA probe kit (manufactured by Abbott). This FISH score was calculated according to the procedure described in the document attached to HER2 gene kit, Pathvysion™ HER2 DNA probe kit (Abbot Japan).

After the tissue array slide was deparaffinized, it was washed with water and autoclaved in a 10 mM citrate buffer (pH 6.0) for 15 minutes to activate the antigen. The tissue array slide after antigen retrieval treatment was washed with a PBS buffer, and anti-HER2 rabbit monoclonal antibody (4B5) diluted to 0.05 nM with a 1% BSA-containing PBS buffer was reacted with the tissue section for 2 hours. After washing with PBS, it was reacted with a biotin-labeled anti-rabbit antibody diluted with a 1% BSA-containing PBS buffer for 30 minutes. Furthermore, using the staining agent for tissue staining, that is, reacting with the luminescent labeling material for pathological diagnosis (luminescent nanoparticles having streptavidin) produced above for 2 hours, followed by washing, an immunohistochemical stained section was obtained. The obtained immunohistochemically stained section was fixed by immersing that in a 4% neutral paraformaldehyde aqueous buffer solution for 10 minutes.

### [Morphological Staining]

HE staining was performed on the immunohistochemically stained section fixed as described above, and the stained section was dehydrated by immersion in ethanol, and the dehydrated section was further cleared by immersing in xylene and air-drying, resulting in a double-stained section.

### [Sealing]

Entellan™ new (manufactured by Merck & Co., Ltd.), which is a xylene-based mounting medium, was added dropwise to the morphologically stained specimen, and the specimen was covered with a cover glass and sealed.

### <Evaluation of Tissue Samples>

A luminescent labeling material for pathological diagnosis in which the HER2 protein expressed on the cell membrane is labeled by specifying the shape of the cell (the position of the cell membrane) by image processing using the stained image for morphological observation and superimposing it on the immunostained image. A bright spot representing the material (streptavidin-modified luminescent nanoparticles composed of luminescent nanoparticles) could be confirmed by microscopic observation by irradiation with excitation light. This result showed that the luminescent nanoparticles of the present invention can be used as a luminescent labeling material for pathological diagnosis.

### INDUSTRIAL APPLICABILITY

The present invention may be used for luminescent nanoparticles and luminescent labeling materials for pathological diagnosis for enabling high-sensitivity imaging that can avoid adverse effects of autofluorescence of cells on bioimaging.

1. Luminescent nanoparticles containing a luminescent dye, wherein the luminescent dye is a compound having a structure represented by the following Formula (1), and the luminescent nanoparticles have at least one of delayed luminescence or long Stokes shift luminescence. 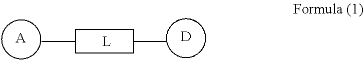
in Formula (1), A represents an electron-withdrawing group; D represents an electron-donating group; L represents a linking group having a backbone containing 2 to 4 atoms; the 2 to 4 atoms are a carbon atom, an oxygen atom, a nitrogen atom, a silicon atom, a sulfur atom, a boron atom, or a phosphorus atom; and the linking group may have a hydrogen atom or a substituent.

2. The luminescent nanoparticle according to claim 1, wherein Formula (1) is represented by at least one of the following Formulas (2) to (6), 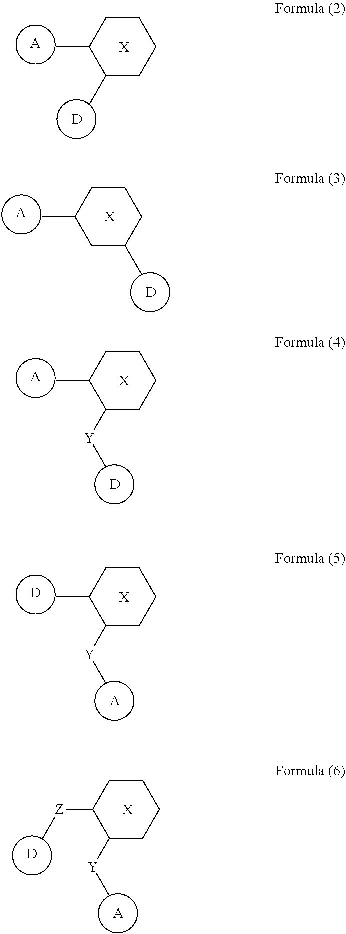
in Formulas (1) to (6), A represents an electron-withdrawing group; D represents an electron-donating group; X represents a cyclic saturated hydrocarbon group, a cyclic unsaturated hydrocarbon group, an aryl group or a heteroaryl group; Y and Z represent a carbon atom, an oxygen atom, a nitrogen atom, a silicon atom, a sulfur atom, a boron atom, or a phosphorus atom and may have a hydrogen atom or a substituent; and Y in Formulas (4) and (5) may be a linking group having a backbone containing two atoms.

3. The luminescent nanoparticles according to claim 1, having delayed luminescence.

4. The luminescent nanoparticles according to claim 1, having intramolecular exciplex luminescence.

5. The luminescent nanoparticles according to claim 1, having long Stokes shift luminescence.

6. The luminescent nanoparticles according to claim 2, wherein in Formulas (2) to (6), A represents “a carbonyl group which may be substituted”, “a sulfonyl group which may be substituted”, “a boryl group which may be substituted”, “a phosphine oxide group which may be substituted”, “an aryl group which may be substituted with an electron-withdrawing group”, “an electron-donating heterocyclic group substituted with a cyano group, a fluorine atom, an alkyl group substituted with a fluorine atom, or a nitrogen-containing aromatic six-membered ring-containing heterocyclic group”, or “an electron-withdrawing heterocyclic group which may be substituted”; D represents “an aryl group substituted with an electron-donating group”, “an electron-donating heterocyclic group which may be substituted with a substituent other than a cyano group, a fluorine atom, an alkyl group substituted with a fluorine atom, or a nitrogen-containing aromatic six-membered ring-containing heterocyclic group”, or “an amino group which may be substituted”; and X represents an aryl group or a heteroaryl group.

7. The luminescent nanoparticles according to claim 2, wherein in Formulas (4) to (6), A represents “a carbonyl group which may be substituted”, “a sulfonyl group which may be substituted”, “a boryl group which may be substituted”, “a phosphine oxide group which may be substituted”, “an aryl group which may be substituted with an electron-withdrawing group”, “an electron-donating heterocyclic group substituted with a cyano group, a fluorine atom, an alkyl group substituted with a fluorine atom, or a nitrogen-containing aromatic six-membered ring-containing heterocyclic group”, or “an electron-withdrawing heterocyclic group which may be substituted”; D represents “an aryl group substituted with an electron-donating group”, “an electron-donating heterocyclic group which may be substituted with a substituent other than a cyano group, a fluorine atom, an alkyl group substituted with a fluorine atom, or a nitrogen-containing aromatic six-membered ring-containing heterocyclic group”, or “an amino group which may be substituted”; X represents an aryl group or a heteroaryl group; and Y represents a carbon atom, an oxygen atom, or a silicon atom.

8. The luminescent nanoparticles according to claim 1, wherein the luminescent dye has a hydrophilic group.

9. The luminescent nanoparticles according to claim 1, containing a binder.

10. The luminescent nanoparticles according to claim 9, wherein the binder present on a surface of the luminescent nanoparticle among the binder has a hydrophilic group.

11. The luminescent nanoparticles according to claim 9, wherein the binder and the luminescent dye form a covalent bond.

12. A luminescent labeling material for pathological diagnosis, wherein a target-directed ligand is covalently bonded to a surface of the luminescent nanoparticle according to claim 1.
